(19) United States
(12) Patent Application Publication
(10) Pub. No.: US 2022/0125480 A1
(43) Pub. Date: Apr. 28, 2022

(54) SYSTEMS, DEVICES, AND METHODS FOR ANALYTE SENSOR APPLICATORS

(71) Applicant: ABBOTT DIABETES CARE INC., Alameda, CA (US)

(72) Inventors: Vivek S. Rao, Alameda, CA (US); Anthony Lin Chern, Alameda, CA (US); Phillip W. Carter, Oakland, CA (US); Joshua Lindsay, Woodside, CA (US); Tuan Nguyen, San Jose, CA (US)

(21) Appl. No.: 17/399,857

(22) Filed: Aug. 11, 2021

Related U.S. Application Data
(60) Provisional application No. 63/072,730, filed on Aug. 31, 2020.

(51) Int. Cl. A61B 17/34; A61B 5/145; A61B 5/00

(52) U.S. Cl. CPC A61B 17/3468; A61B 2017/00831; A61B 5/0022; A61B 5/14532

(57) ABSTRACT

An assembly and method for delivery of an analyte sensor including a reusable applicator having a proximal portion and a distal portion are disclosed. The reusable applicator can include a housing, a sensor carrier configured a sensor carrier configured to releasably receive a first analyte sensor, a sharp carrier configured to releasably receive a sharp module and movable between the proximal portion of the reusable applicator and the distal portion of the reusable applicator for delivery of the first analyte sensor from the reusable applicator, and a reset tool configured to reset the reusable applicator for delivery of another analyte sensor.

### CROSS-REFERENCE TO RELATED APPLICATIONS

This application is claims priority to and the benefit of U.S. Provisional Application No. 63/072,730, filed Aug. 31, 2020, which is incorporated by reference herein in its entirety for all purposes.

### FIELD

The subject matter described herein relates generally to systems, devices, and methods of using an applicator to insert at least a portion of an analyte sensor in a subject.

### BACKGROUND

The detection and/or monitoring of analyte levels, such as glucose, ketones, lactate, oxygen, hemoglobin Al C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While current sensors can be convenient for users, they are also susceptible to malfunctions. These malfunctions can be caused by user error, lack of proper training, poor user coordination, overly complicated procedures, physiological responses to the inserted sensor, and other issues. Some prior art systems, for example, may rely too much on the precision assembly and deployment of a sensor control device and an applicator by the individual user. Other prior art systems may utilize sharp insertion and retraction mechanisms that are susceptible to trauma to the surrounding tissue at the sensor insertion site, which can lead to inaccurate analyte level measurements. These challenges and others described herein can lead to improper insertion and/or suboptimal analyte measurements by the sensor, and consequently, a failure to properly monitor the patient's analyte level.

Moreover, applicators used to insert at least a portion of an in vivo analyte sensors can include several components that are often constructed of a mixture of plastic materials, which can be difficult to separate after use making recycling difficult. Additionally, packaging materials for such applicators must fulfill a number of engineering design requirements, including, providing stringent sealing for shelf life storage requirements that demand tight tolerance components with exotic plastic materials for low moisture vapor transition rate, providing adequate lubricity so that insertion force can be maintained, etc. Furthermore, applicators are often packaged inside a carton with alcohol wipes. As a result, applicators are often manufactured for single use and using non-biodegradable materials making them difficult to recycle and/or not durable enough for reuse.

Thus, a need exists for more reliable sensor insertion devices, systems and methods, that are easy to use by the patient, less prone to error, and reusable. Furthermore, a need exists for an applicator that meets engineering design requirements yet can be used multiple times and/or can be recycled.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to an assembly for delivery of an analyte sensor including a reusable applicator configured for delivery of a first analyte sensor and a reset tool configured to reset the reusable applicator for delivery of another analyte sensor. The reusable applicator includes a proximal portion and a distal portion, a sensor carrier configured to releasably receive a first analyte sensor, and a sharp carrier configured to releasably receive a sharp module and movable between the proximal portion of the reusable applicator and the distal portion of the reusable applicator for delivery of the first analyte sensor.

According to certain embodiments of the present disclosure, the reusable applicator can further include a sheath configured to be movable between the proximal portion of the reusable applicator and the distal portion of the reusable applicator, and the reset tool can include a first longitudinal length having a first section having a first traverse dimension configured to be inserted into the sharp carrier of the reusable applicator to release the sharp module and a second section having a second traverse dimension configured to be inserted into the sheath of the reusable applicator to move the sharp carrier from the proximal portion of the reusable applicator toward the distal portion of the reusable applicator.

According to certain embodiments of the present disclosure, the reset tool can include a second longitudinal length having a third traverse dimension configured to be inserted into the reusable applicator to move the sheath from the proximal portion of the reusable applicator toward the distal portion of the reusable applicator. The first longitudinal length of reset tool can be telescopically coupled to the second longitudinal length. The second longitudinal length of the reset tool can include a handle portion. The third traverse dimension of reset tool can be larger than the second traverse dimension, and the second traverse dimension is larger than the first traverse dimension. The second longitudinal length of the reset tool can house a spring.

According to embodiments of the present disclosure, the assembly can include a docking station including a recess for releasably position another analyte sensor and a collection chamber to collect the sharp module. The docking station can include a first channel to collect a sharp module and a second channel to releasably position another analyte sensor.

According to embodiments of the present disclosure, the reusable applicator can include a removable plug to access a reset channel. The reusable applicator is made of a recyclable material, such as acetal. The assembly can include a sealable container having a low moisture vapor transition rate to package the reusable applicator.

According to embodiments of the present disclosure, the assembly can include an applicator cap sealingly coupled to the housing with a gasketless seal.

According to embodiments of the present disclosure, a method for delivery of an analyte sensor includes providing a reusable applicator having a proximal portion and a distal portion, a housing, a sensor carrier having a first analyte sensor releasably received therein, and a sharp carrier having a sharp module releasably received therein. The method further includes moving the sharp carrier from the proximal portion of the reusable applicator toward the distal portion of the reusable applicator to deliver a first analyte sensor from the reusable applicator, and using a reset tool to reset the reusable applicator for delivery of another analyte sensor. The method can include delivering another analyte sensor from a reusable applicator.

According to embodiments of the present disclosure, using the reset tool can include: inserting the reset tool within a reset channel of the reusable applicator; advancing the reset tool to release the sharp module releasably received within the sharp carrier of the reusable applicator; advancing the reset tool to compress a return spring of the reusable applicator by moving the sharp carrier of the reusable applicator from the proximal portion of the reusable applicator toward the distal portion of the reusable applicator; and advancing the reset tool to move a sheath of the reusable applicator from the proximal portion of the reusable applicator toward the distal portion of the reusable applicator.

According to embodiments of the present disclosure, the method for delivery of an analyte sensor can include advancing the reusable applicator into a first channel of a docking station including a collection chamber to collect the sharp module, releasing the sharp module into the collection chamber., advancing the reusable applicator into a second channel of the docking station releasably positioning another analyte sensor, and coupling the another analyte sensor to the sensor carrier. The method for delivery of an analyte sensor can include advancing the reusable applicator into a channel of a docking station, the channel releasably positioning another sensor and the docking station including a collection chamber to collect the sharp module, coupling the second sensor control device to the sensor carrier, and releasing the sharp module into the collection chamber.

According to embodiments of the present disclosure, the method for delivery of an analyte sensor can include removing a removable plug to access the reset channel. The method for delivery of an analyte sensor can include packaging the reusable applicator into a sealable container for shipment. The method for delivery of an analyte sensor can include removing an applicator cap form the housing, wherein applicator cap can be sealingly coupled to the housing with a gasketless seal.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIGS. 3A to 3G are progressive views of an example embodiment of the assembly and application of the system of FIG. 1 incorporating a two-piece architecture.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is side view depicting an example embodiment of a housing.

FIG. 7B is a perspective view depicting an example embodiment of a distal end of a housing.

FIG. 7C is a side cross-sectional view depicting an example embodiment of a housing.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIG. 8F is a perspective view depicting an example embodiment of a compressible distal end of an applicator.

FIGS. 8G to 8K are cross-sectional views depicting example geometries for embodiments of compressible distal ends of an applicator.

FIG. 8L is a perspective view of an example embodiment of an applicator having a compressible distal end.

FIG. 8M is a cross-sectional view depicting an example embodiment of an applicator having a compressible distal end.

FIG. 9A is a proximal perspective view depicting an example embodiment of a sensor carrier.

FIG. 9B is a distal perspective view depicting an example embodiment of a sensor carrier.

FIG. 10 is a proximal perspective view of an example embodiment of a sharp carrier.

FIG. 11 is a side cross-section depicting an example embodiment of a sharp carrier.

FIGS. 12A to 12B are top and bottom perspective views, respectively, depicting an example embodiment of a sensor module.

FIGS. 13A and 13B are perspective and compressed views, respectively, depicting an example embodiment of a sensor connector.

FIG. 14 is a perspective view depicting an example embodiment of a sensor.

FIGS. 15A and 15B are bottom and top perspective views, respectively, of an example embodiment of a sensor module assembly.

FIGS. 16A and 16B are close-up partial views of an example embodiment of a sensor module assembly.

FIG. 16C is a side view of an example sensor, according to one or more embodiments of the disclosure.

FIGS. 17A and 17B are isometric and partially exploded isometric views of an example connector assembly, according to one or more embodiments.

FIG. 17C is an isometric bottom view of the connector of FIGS. 17A-17B.

FIGS. 17D and 17E are isometric and partially exploded isometric views of another example connector assembly, according to one or more embodiments.

FIG. 17F is an isometric bottom view of the connector of FIGS. 17D-17E.

FIG. 18A is a perspective view depicting an example embodiment of a sharp module.

FIG. 18B is a perspective view depicting an example embodiment of a sharp module.

FIGS. 18C and 18D are a side view and a perspective view depicting another example embodiment of a sharp module.

FIG. 18E is a cross-sectional view depicting an example embodiment of an applicator.

FIG. 18F is a flow diagram depicting an example embodiment method for sterilizing an applicator assembly.

FIGS. 18G and 18H are photographs depicting example embodiments of sharp tips.

FIGS. 18I and 18J are perspective views depicting example embodiments of sharp modules.

FIGS. 19A and 19B are isometric and side views, respectively, of another example sensor control device.

FIGS. 20A and 20B are exploded isometric top and bottom views, respectively of the sensor control device of FIGS. 19A-19B.

FIG. 21 is a cross-sectional side view of an assembled sealed subassembly, according to one or more embodiments.

FIGS. 22A-22C are progressive cross-sectional side views showing assembly of the sensor applicator with the sensor control device of FIGS. 19A-19B.

FIGS. 23A and 23B are perspective and top views, respectively, of the cap post of FIG. 22C, according to one or more additional embodiments.

FIG. 24 is a cross-sectional side view of the sensor control device of FIGS. 19A-19B.

FIGS. 25A and 25B are cross-sectional side views of the sensor applicator ready to deploy the sensor control device to a target monitoring location.

FIGS. 26A-26C are progressive cross-sectional side views showing assembly and disassembly of an example embodiment of the sensor applicator with the sensor control device of FIGS. 19A-19B.

FIG. 27A is an isometric bottom view of the housing, according to one or more embodiments.

FIG. 28A is an isometric bottom view of the housing with the sheath and other components at least partially positioned therein.

FIG. 29 is an enlarged cross-sectional side view of the sensor applicator with the sensor control device installed therein, according to one or more embodiments.

FIG. 30A is an isometric top view of the cap, according to one or more embodiments.

FIG. 30B is an enlarged cross-sectional view of the engagement between the cap and the housing, according to one or more embodiments.

FIGS. 31A and 31B are isometric views of the sensor cap and the collar, respectively, according to one or more embodiments.

FIGS. 32A and 32B are side and isometric views, respectively, of an example sensor control device, according to one or more embodiments of the present disclosure.

FIGS. 33A and 33B are exploded, isometric top and bottom views, respectively, of the sensor control device of FIG. 2, according to one or more embodiments.

FIG. 34 is a cross-sectional side view of the sensor control device of FIGS. 32A-32B and 33A-33B, according to one or more embodiments.

FIG. 34A is an exploded isometric view of a portion of another embodiment of the sensor control device of FIGS. 32A-32B and 33A-33B.

FIG. 35A is an isometric bottom view of the mount of FIGS. 32A-32B and 33A-33B.

FIG. 35B is an isometric top view of the sensor cap of FIGS. 32A-32B and 33A-33B.

FIGS. 36A and 36B are side and cross-sectional side views, respectively, of an example sensor applicator, according to one or more embodiments.

FIGS. 37A and 37B are perspective and top views, respectively, of the cap post of FIG. 36B, according to one or more embodiments.

FIG. 38 is a cross-sectional side view of the sensor control device positioned within the applicator cap, according to one or more embodiments.

FIG. 39 is a cross-sectional view of a sensor control device showing example interaction between the sensor and the sharp.

FIGS. 40A-40F illustrate cross-sectional views depicting an example embodiment of an applicator during a stage of deployment.

FIGS. 41A-B are enlarged cross-sectional side views of the interface between applicator housing and applicator cap.

FIGS. 41C-D are enlarged cross-sectional side views of an applicator housing and applicator cap.

FIG. 41E is a chart reflecting certain characteristics of example embodiments of materials and seals used for packaging.

FIGS. 42A-42O are perspective top and cross-sectional views depicting an example embodiment of an applicator, reset tool, and docking station during various stages of resetting.

FIG. 43A-D perspective views depicting an example embodiment of an applicator, reset tool, and docking station during various stages of resetting.

FIG. 44 is a perspective view depicting an example embodiment of a docking station.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed, and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for the improved assembly and use of analyte sensor insertion devices for use with in vivo analyte monitoring systems. In particular, several embodiments of the present disclosure are designed to improve the method of sensor insertion with respect to in vivo analyte monitoring systems and, in particular, to minimize trauma to an insertion site during a sensor insertion process. Some embodiments, for example, include a powered sensor insertion mechanism configured to operate at a higher, controlled speed relative to a manual insertion mechanism, in order to reduce trauma to an insertion site. In other embodiments, an applicator having a compressible distal end can stretch and flatten the skin surface at the insertion site, and consequently, can reduce the likelihood of a failed insertion as a result of skin tenting. In still other embodiments, a sharp with an offset tip, or a sharp manufactured utilizing a plastic material or a coined manufacturing process can also reduce trauma to an insertion site. In sum, these embodiments can improve the likelihood of a successful sensor insertion and reduce the amount of trauma at the insertion site, to name a few advantages.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

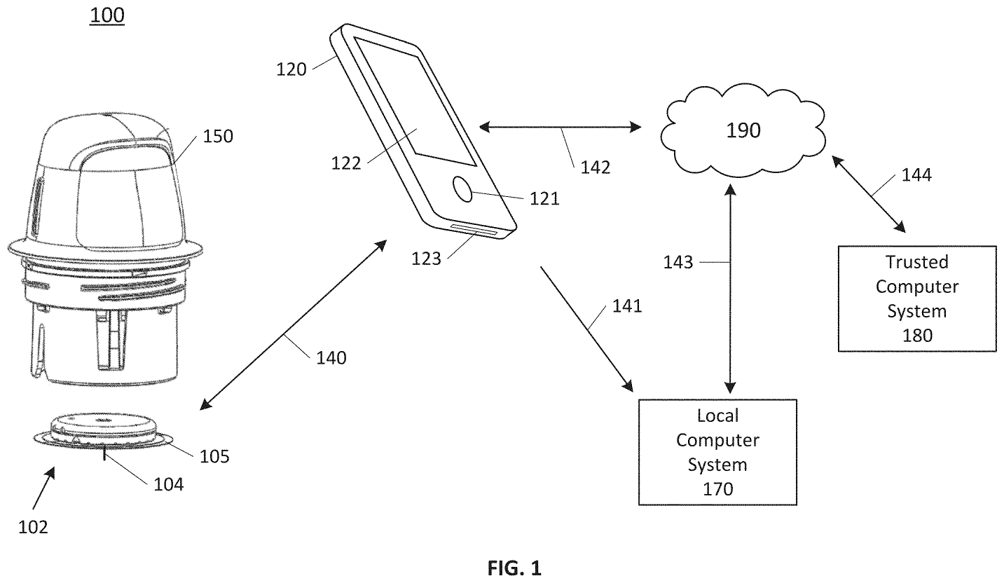

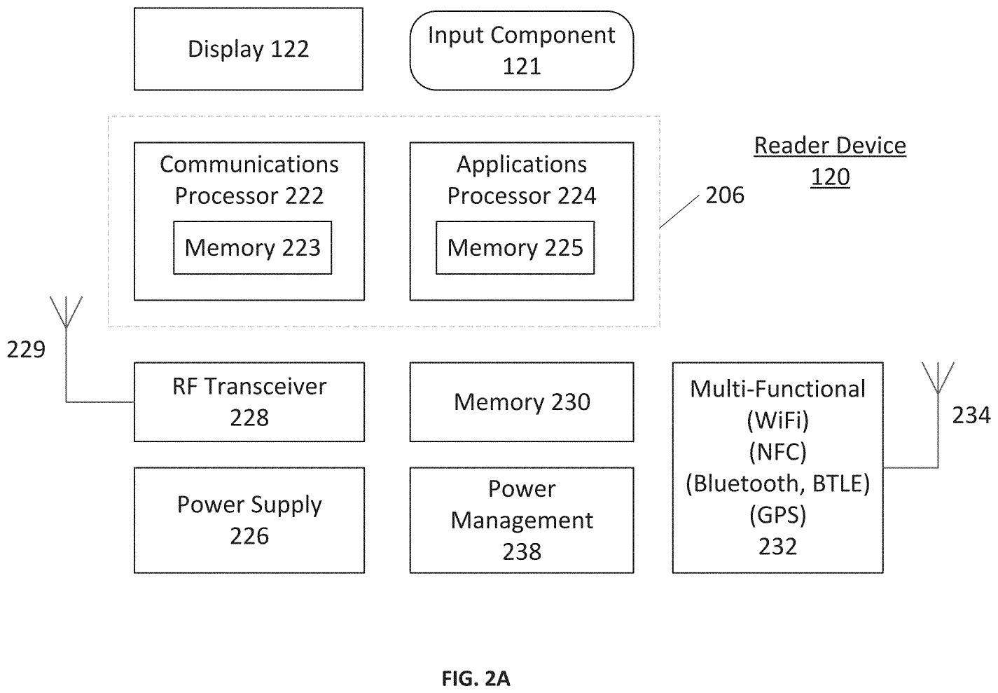

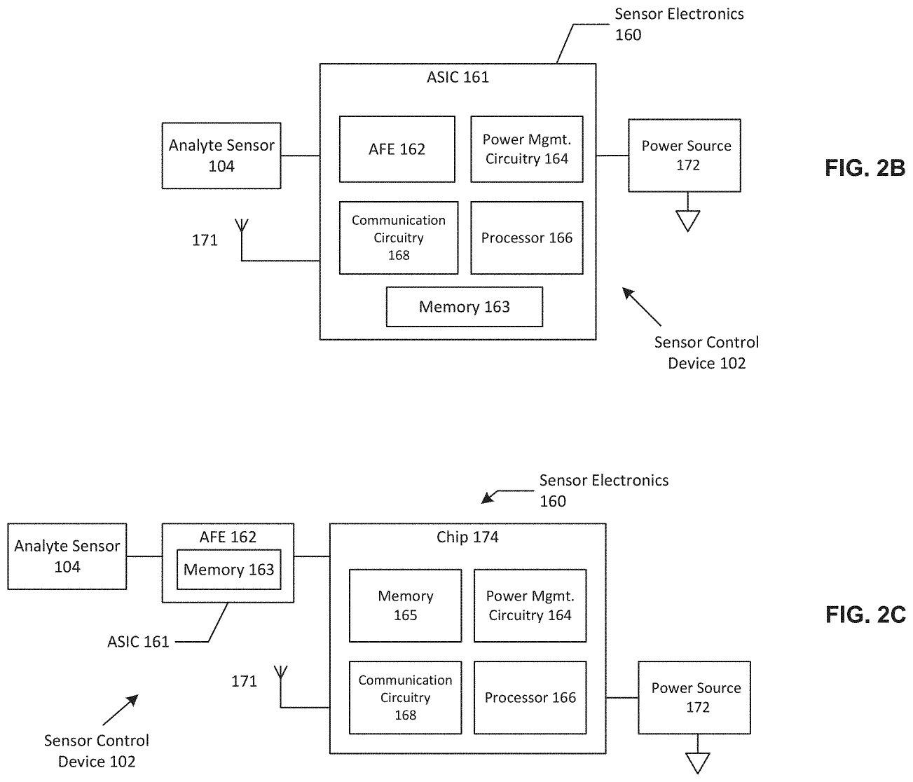

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121, and the device battery can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiment of Reader Device

FIG. 2A is a block diagram depicting an example embodiment of a reader device 120 configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Assembly Processes for Sensor Control Device

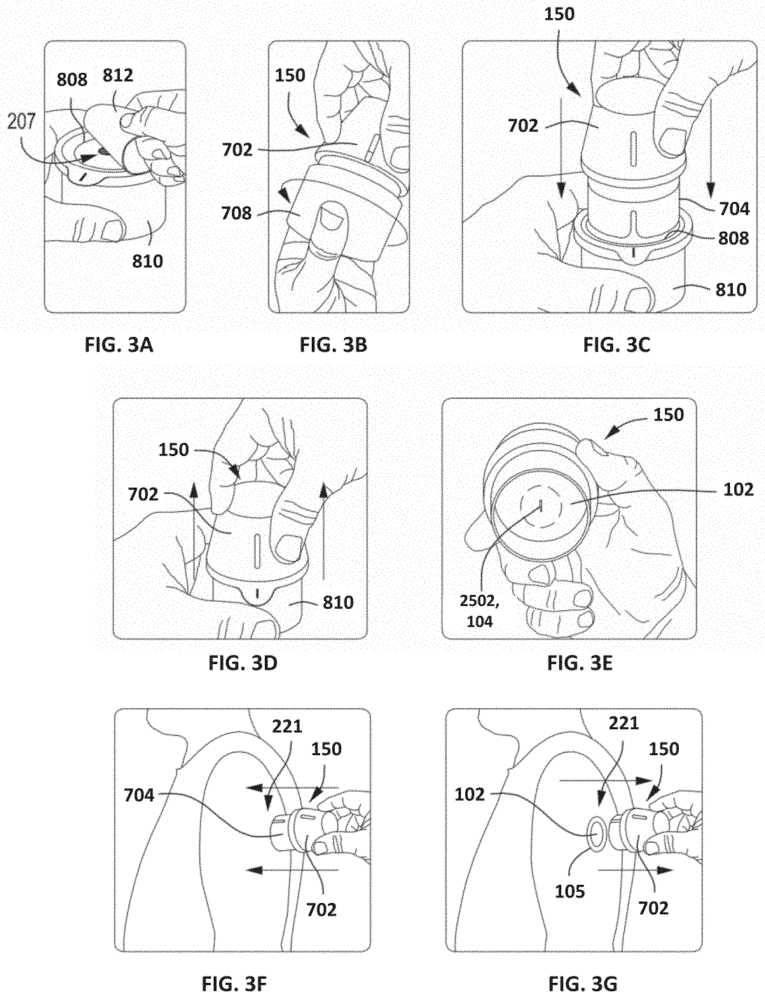

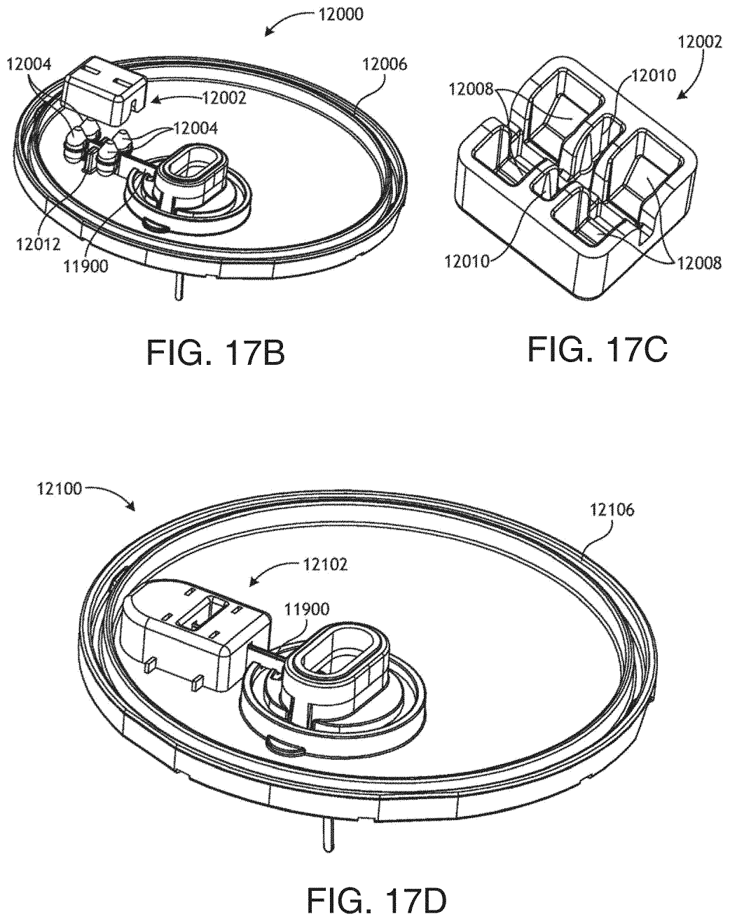

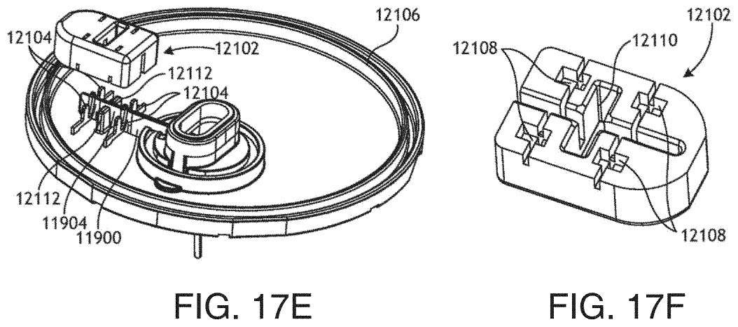

According to some embodiments, the components of sensor control device 102 can be acquired by a user in multiple packages requiring final assembly by the user before delivery to an appropriate user location. FIGS. 3A-3E depict an example embodiment of an assembly process for sensor control device 102 by a user, including preparation of separate components before coupling the components in order to ready the sensor for delivery. In other embodiments, such as those described with respect to FIGS. 17B to 17F, components of the sensor control device 102 and applicator 150 can be acquired by a user in a single package. FIGS. 3F-3G depict an example embodiment of delivery of sensor control device 102 to an appropriate user location by selecting the appropriate delivery location and applying device 102 to the location.

FIG. 3A depicts a sensor container or tray 810 that has a removable lid 812. The user prepares the sensor tray 810 by removing the lid 812, which acts as a sterile barrier to protect the internal contents of the sensor tray 810 and otherwise maintain a sterile internal environment. Removing the lid 812 exposes a platform 808 positioned within the sensor tray 810, and a plug assembly 207 (partially visible) is arranged within and otherwise strategically embedded within the platform 808. The plug assembly 207 includes a sensor module (not shown) and a sharp module (not shown). The sensor module carries the sensor 104 (FIG. 1), and the sharp module carries an associated sharp used to help deliver the sensor 104 transcutaneously under the user's skin during application of the sensor control device 102 (FIG. 1).

FIG. 3B depicts the sensor applicator 150 and the user preparing the sensor applicator 150 for final assembly. The sensor applicator 150 includes a housing 702 sealed at one end with an applicator cap 708. In some embodiments, for example, an O-ring or another type of sealing gasket may seal an interface between the housing 702 and the applicator cap 708. In at least one embodiment, the O-ring or sealing gasket may be molded onto one of the housing 702 and the applicator cap 708. The applicator cap 708 provides a barrier that protects the internal contents of the sensor applicator 150. In particular, the sensor applicator 150 contains an electronics housing (not shown) that retains the electrical components for the sensor control device 102 (FIG. 1), and the applicator cap 708 may or may not maintain a sterile environment for the electrical components. Preparation of the sensor applicator 150 includes uncoupling the housing 702 from the applicator cap 708, which can be accomplished by unscrewing the applicator cap from the housing 702. The applicator cap 708 can then be discarded or otherwise placed aside.

FIG. 3C depicts the user inserting the sensor applicator 150 into the sensor tray 810. The sensor applicator 150 includes a sheath 704 configured to be received by the platform 808 to temporarily unlock the sheath 704 relative to the housing 702, and also temporarily unlock the platform 808 relative to the sensor tray 810. Advancing the housing 702 into the sensor tray 810 results in the plug assembly 207 (FIG. 3A) arranged within the sensor tray 810, including the sensor and sharp modules, being coupled to the electronics housing arranged within the sensor applicator 150.

In FIG. 3D, the user removes the sensor applicator 150 from the sensor tray 810 by proximally retracting the housing 702 with respect to the sensor tray 810.

FIG. 3E depicts the bottom or interior of the sensor applicator 150 following removal from the sensor tray 810 (FIGS. 3A and 3C). The sensor applicator 150 is removed from the sensor tray 810 with the sensor control device 102 fully assembled therein and positioned for delivery to the target monitoring location. As illustrated, a sharp 2502 extends from the bottom of the sensor control device 102 and carries a portion of the sensor 104 within a hollow or recessed portion thereof. The sharp 2502 is configured to penetrate the skin of a user and thereby place the sensor 104 into contact with bodily fluid.

FIGS. 3F and 3G depict example delivery of the sensor control device 102 to a target monitoring location 221, such as the back of an arm of the user. FIG. 3F shows the user advancing the sensor applicator 150 toward the target monitoring location 221. Upon engaging the skin at the target monitoring location 221, the sheath 704 collapses into the housing 702, which allows the sensor control device 102 (FIGS. 3E and 3G) to advance into engagement with the skin. With the help of the sharp 2502 (FIG. 3E), the sensor 104 (FIG. 3E) is advanced transcutaneously into the patient's skin at the target monitoring location 221.

FIG. 3G shows the user retracting the sensor applicator 150 from the target monitoring location 221, with the sensor control device 102 successfully attached to the user's skin. The adhesive patch 105 (FIG. 1) applied to the bottom of sensor control device 102 adheres to the skin to secure the sensor control device 102 in place. The sharp 2502 (FIG. 3E) is automatically retracted when the housing 702 is fully advanced at the target monitoring location 221, while the sensor 104 (FIG. 3E) is left in position to measure analyte levels.

According to some embodiments, system 100, as described with respect to FIGS. 3A-3G and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes.

Example Embodiment of Sensor Applicator Device

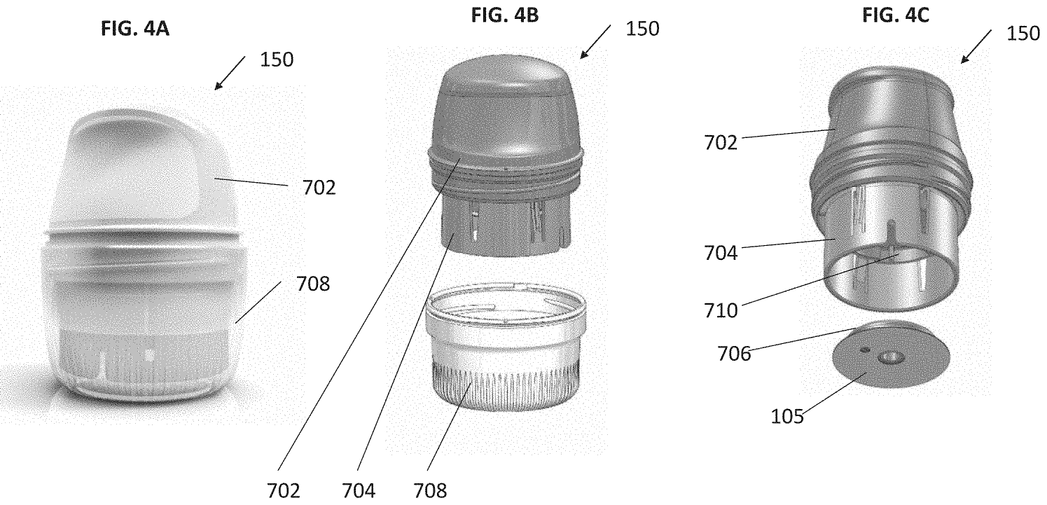

FIG. 4A is a side view depicting an example embodiment of an applicator device 150 coupled with screw cap 708. This is one example of how applicator 150 is shipped to and received by a user, prior to assembly by the user with a sensor. In other embodiments, applicator 150 can be shipped to the user with the sensor and sharp contained therein. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within sensor carrier 710 of sheath 704, when cap 708 is in place.

### Example Embodiment of Tray and Sensor Module Assembly

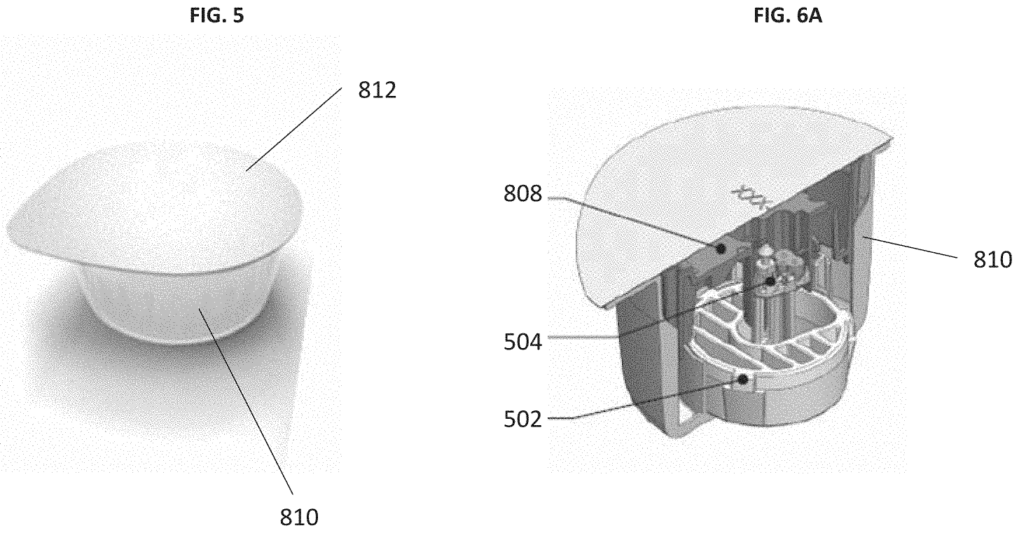

FIG. 5 is a proximal perspective view depicting an example embodiment of a tray 810 with sterilization lid 812 removably coupled thereto, which, in some embodiments, may be representative of how the package is shipped to and received by a user prior to assembly.

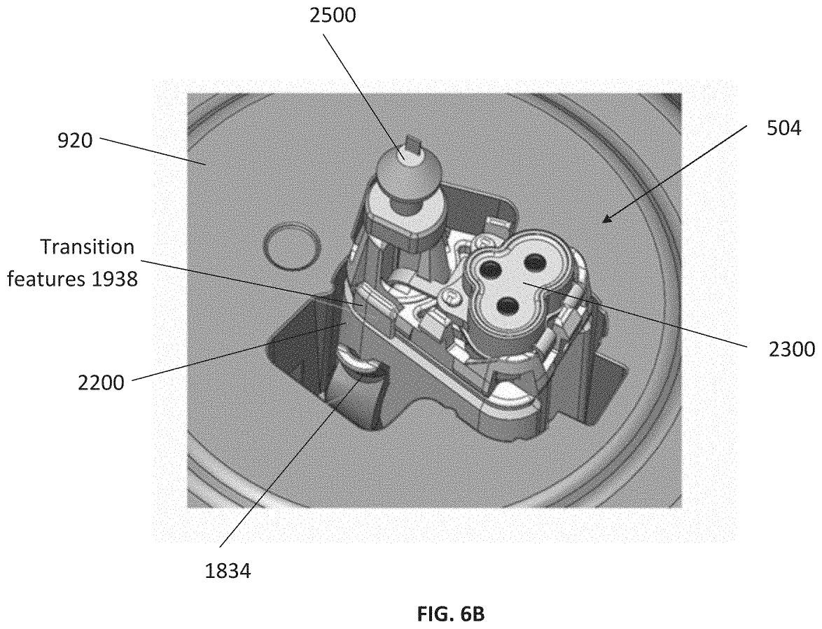

FIG. 6A is a proximal perspective, cutaway view depicting sensor delivery components within tray 810, according to some embodiments. Platform 808 is slidably coupled within tray 810. Desiccant 502 is stationary with respect to tray 810. Sensor module 504 is mounted within tray 810.

FIG. 6B is a proximal perspective view depicting an example embodiment of a sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

### Example Embodiment of Applicator Housing

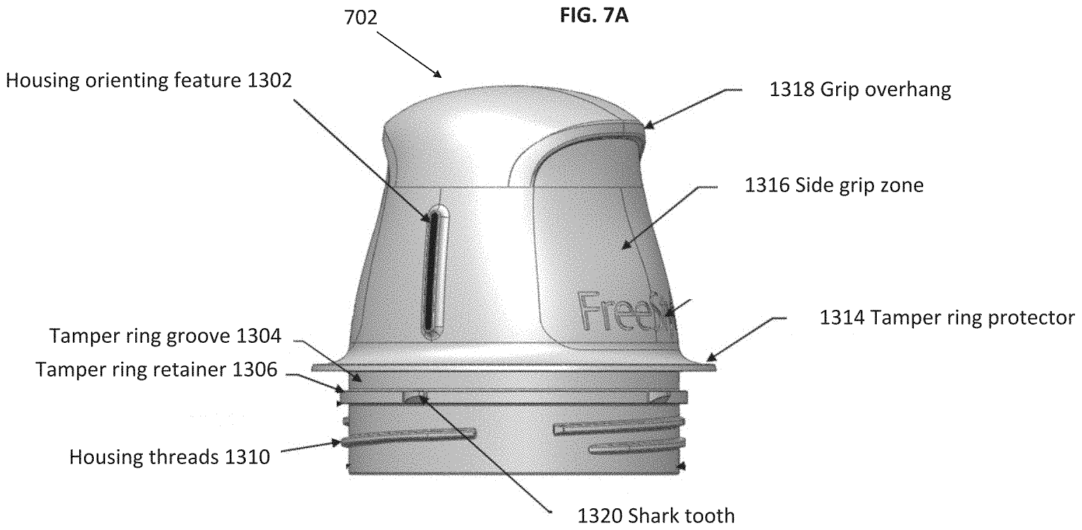

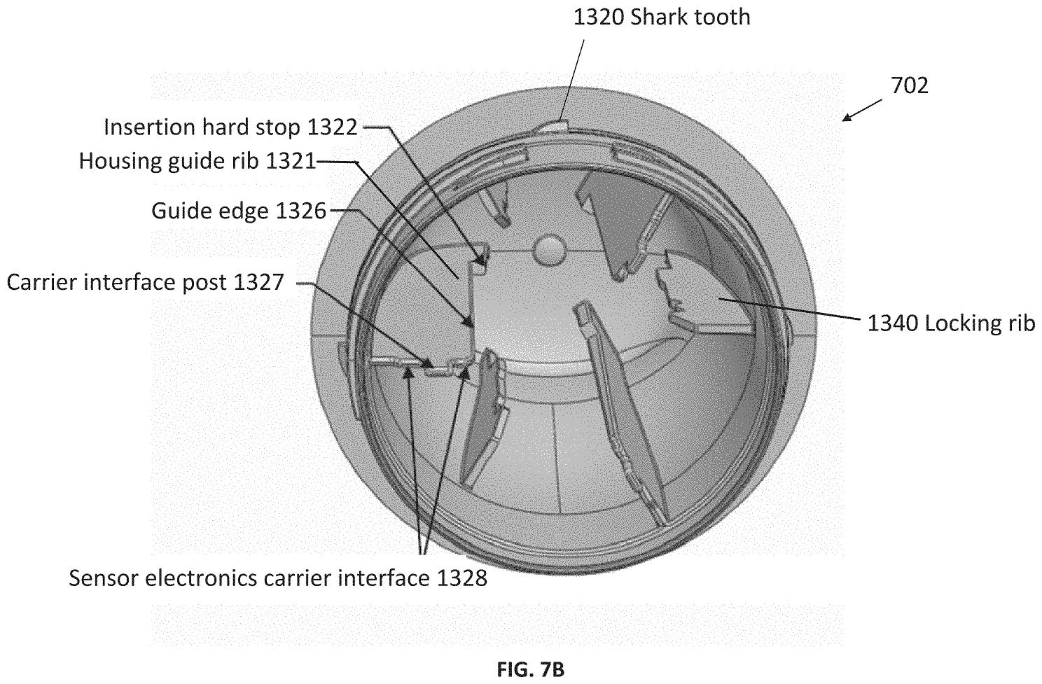

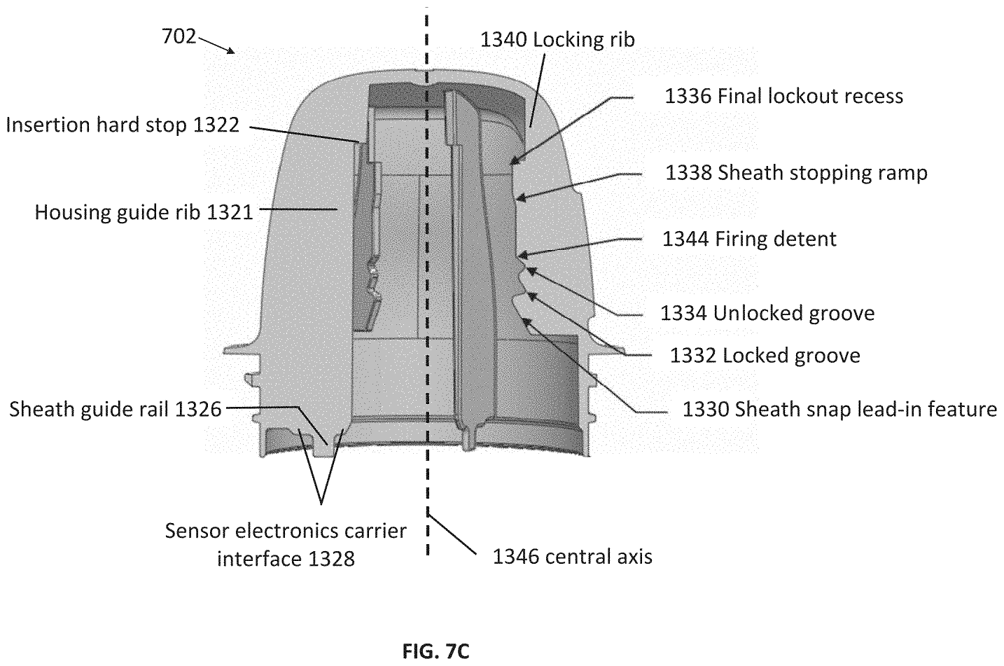

FIG. 7A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in ease of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

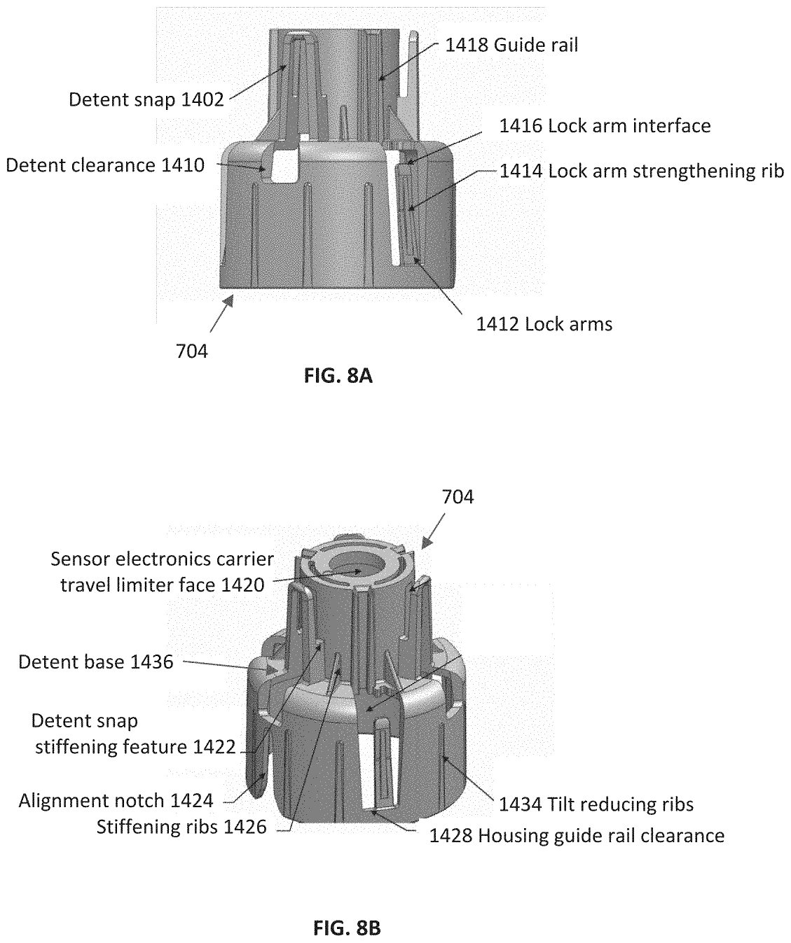

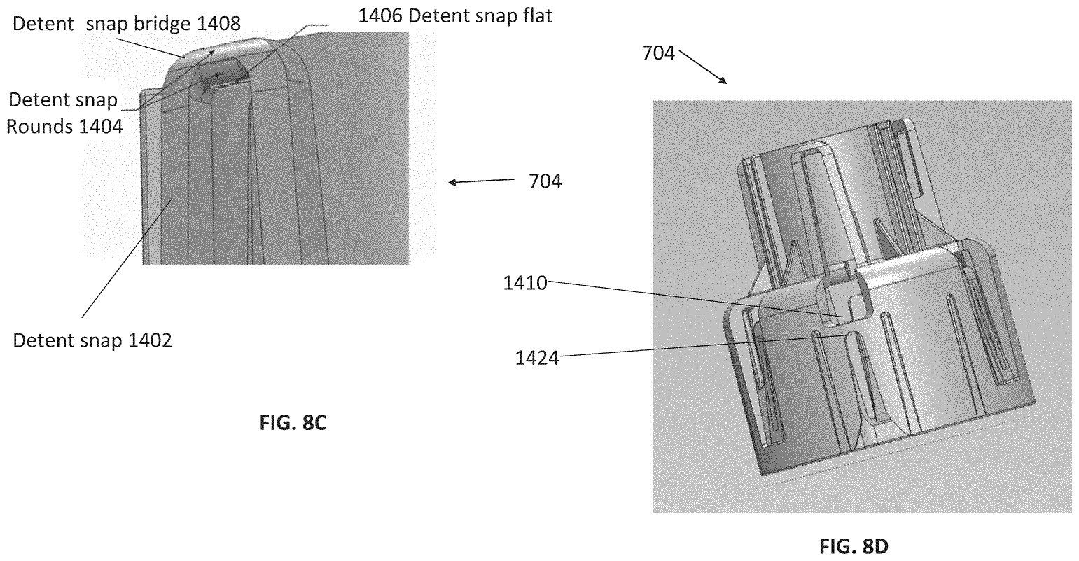

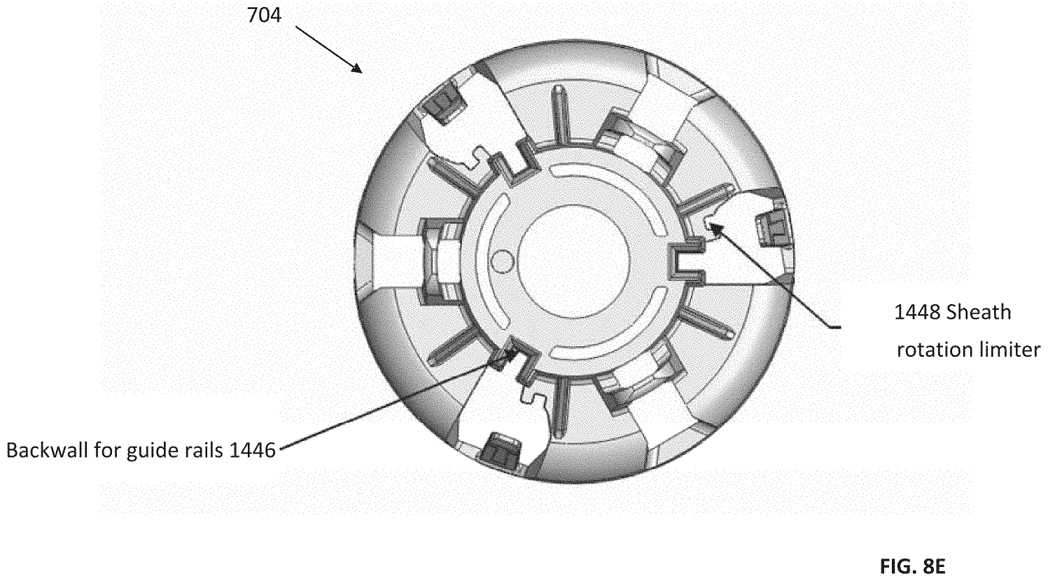

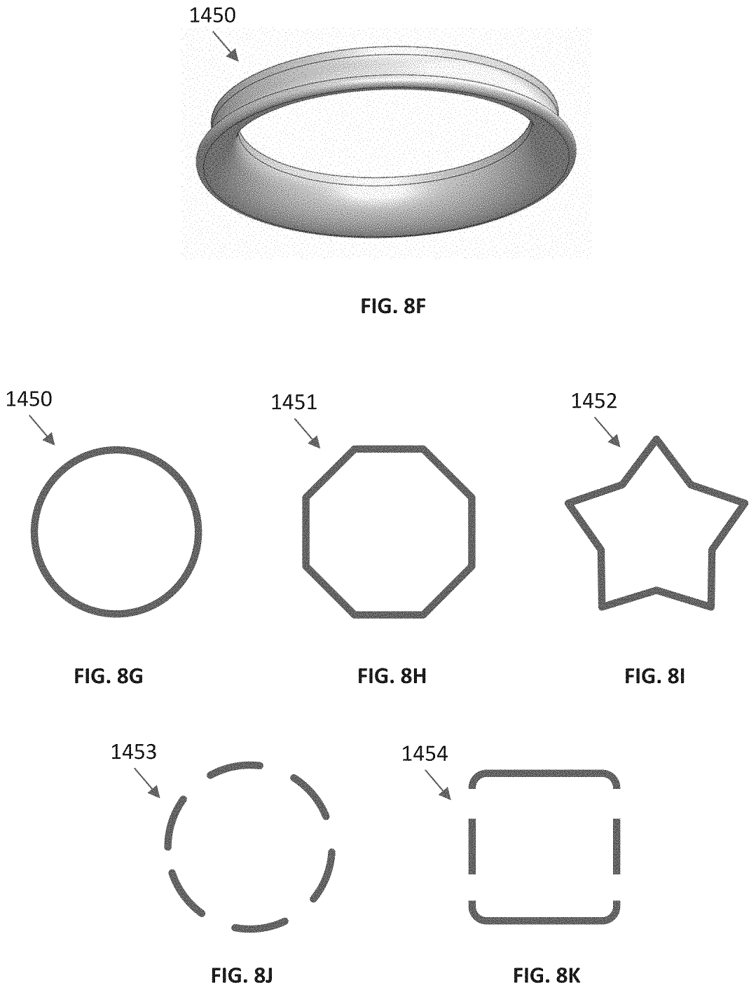

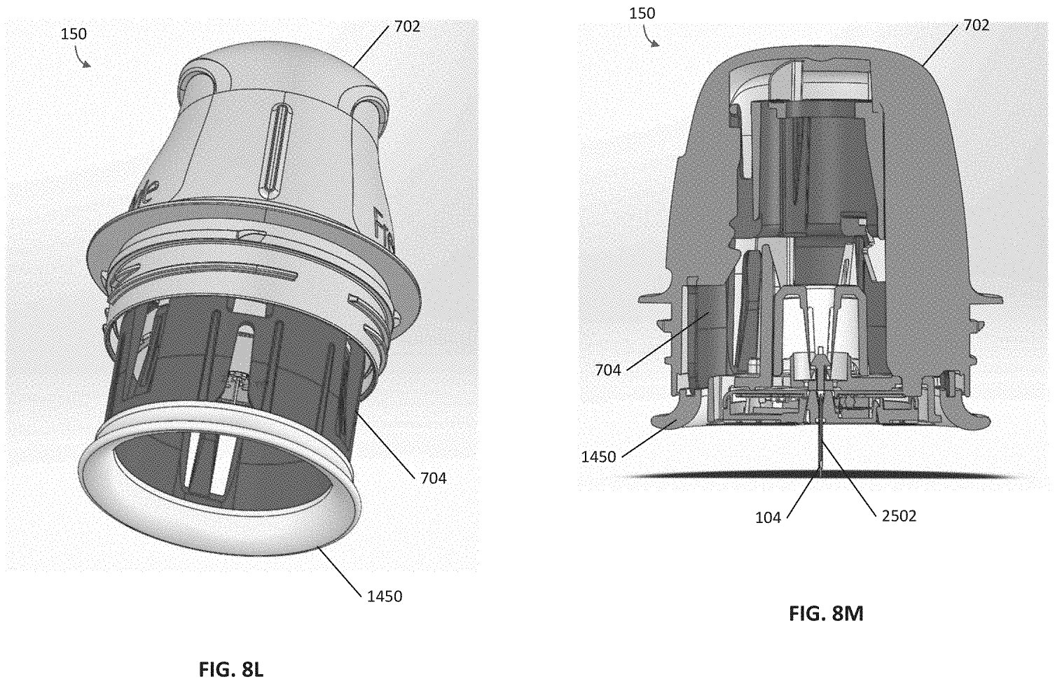

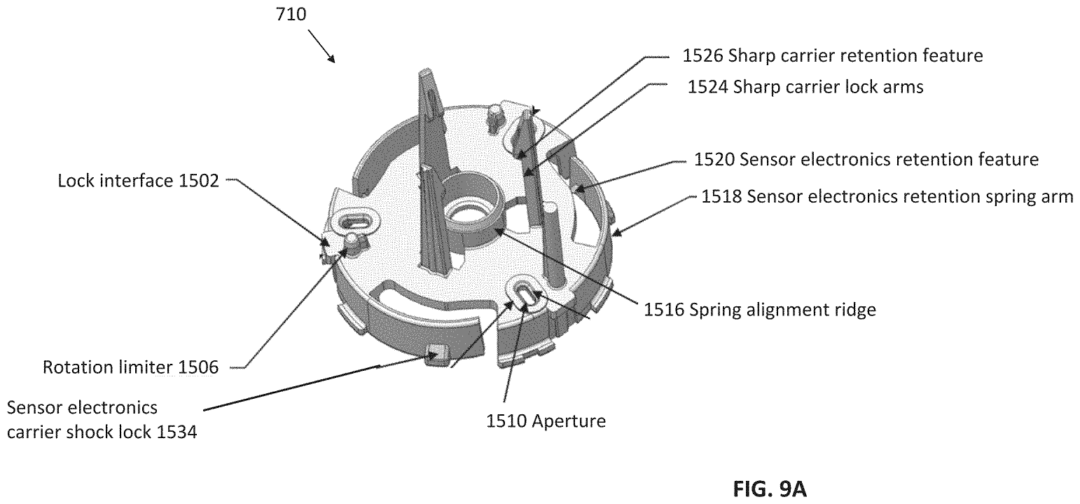

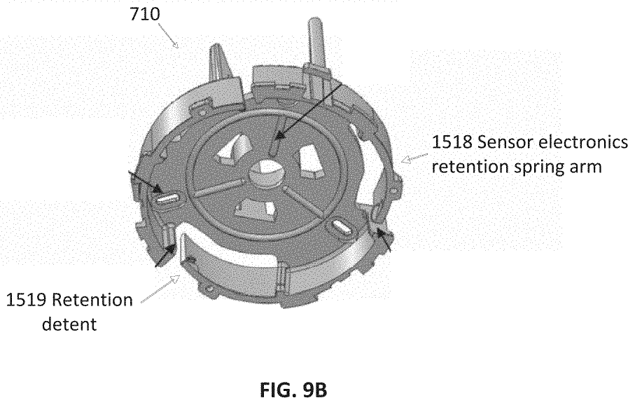

FIG. 7B is a perspective view depicting a distal end of housing 702. Here, three housing guide structures (or “guide ribs”) 1321 are located at 120 degree angles with respect to each other, and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a sensor carrier travel limiter face 1420 of a sheath 704 (FIG. 8B) to abut during use, preventing sensor carrier travel limiter face 1420 from moving any further in a proximal direction. A carrier interface post 1327 passes through an aperture 1510 (FIG. 9A) of sensor carrier 710 during an assembly. A sensor carrier interface 1328 can be a rounded, distally facing surface of housing guide ribs 1321 which interfaces with sensor carrier 710.

FIG. 7C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor carrier travel limiter face 1420.

### Example Embodiment of Applicator Sheath

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock sensor carrier 710 to sheath 704 when lock arm interface 1416 of lock arms 1412 engage lock interface 1502 of sensor carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704.

FIG. 8F is a perspective view depicting an example embodiment of a compressible distal end 1450, which can be attached and/or detached from a sheath 704 of an applicator 150. In a general sense, the embodiments described herein operate by flattening and stretching a skin surface at a predetermined site for sensor insertion. Moreover, the embodiments described herein may also be utilized for other medical applications, such as, e.g., transdermal drug delivery, needle injection, wound closure stitches, device implantation, the application of an adhesive surface to the skin, and other like applications.

By way of background, those of skill the art will appreciate that skin is a highly anisotropic tissue from a biomechanical standpoint and varies largely between individuals. This can affect the degree to which communication between the underlying tissue and the surrounding environment can be performed, e.g., with respect to drug diffusion rates, the ability to penetrate skin with a sharp, or sensor insertion into the body at a sharp-guided insertion site.

In particular, the embodiments described herein are directed to reducing the anisotropic nature of the skin in a predetermined area by flattening and stretching the skin, and thereby improving upon the aforementioned applications. Smoothing the skin (e.g., flattening to remove wrinkles) before mating with a similarly shaped (e.g., a flat, round adhesive pad of a sensor control unit) can produce a more consistent surface area contact interface. As the surface profile of the skin approaches the profile specifications of the designed surface of the device (or, e.g., the designed area of contact for drug delivery), the more consistent contact (or drug dosing) can be achieved. This can also be advantageous with respect to wearable adhesives by creating a continuum of adhesive-to-skin contact in a predetermined area without wrinkles. Other advantages can include (1) an increased wear duration for devices that rely on skin adhesion for functionality, and (2) a more predictable skin contact area, which would improve dosing in transcutaneous drug/pharmaceutical delivery.

In addition, skin flattening (e.g., as a result of tissue compression) combined with stretching can reduce the skin's viscoelastic nature and increase its rigidity which, in turn, can increase the success rate of sharp-dependent sensor placement and functionality.

With respect to sensor insertion, puncture wounds can contribute to early signal aberration (ESA) in sensors and may be mitigated when the skin has been flattened and stretched rigid. Some known methods to minimize a puncture wound include: (1) reducing the introducers' size, or (2) limiting the length of the needle inserted into the body. However, these known methods may reduce the insertion success rate due to the compliance of the skin. For example, when a sharp tip touches the skin, before the tip penetrates the skin, the skin deforms inward into the body, a phenomenon also referred to as “skin tenting.” If the sharp is not stiff enough due to a smaller cross-sectional area and/or not long enough, the sharp may fail to create an insertion point large enough, or in the desired location due to deflection, for the sensor to pass through the skin and be positioned properly. The degree of skin tenting can vary between and within subjects, meaning the distance between a sharp and a skin surface can vary between insertion instances. Reducing this variation by stretching and flattening the skin can allow for a more accurately functioning and consistent sensor insertion mechanism.

Referring to FIG. 8F, a perspective view depicts an example embodiment of a compressible distal end 1450 of an applicator 150. According to some embodiments, compressible distal end 1450 can be manufactured from an elastomeric material. In other embodiments, compressible distal end 1450 can be made of metal, plastic, composite legs or springs, or a combination thereof.

In some embodiments, compressible distal end 1450 can be detachable from an applicator 150 and used with various other similar or dissimilar applicators or medical devices. In other embodiments, compressible distal end 1450 can be manufactured as part of the sheath 704. In still other embodiments, the compressible distal end 1450 can be attached to other portions of applicator 150 (e.g., sensor carrier), or, alternatively, can be used as a separate standalone device. Furthermore, although compressible distal end 1450 is shown in FIGS. 8F and 8G as having a continuous ring geometry, other configurations can be utilized. For example, FIGS. 8H to 8K are cross-sectional views depicting various example compressible distal ends, having an octagonal geometry 1451 (FIG. 8H), star-shaped geometry 1452 (FIG. 81), a non-continuous ring geometry 1453 (FIG. 8J), and a non-continuous rectangular geometry (FIG. 8K). With respect to FIGS. 8J and 8K, a compressible distal end with a non-continuous geometry would have a plurality of points or spans to contact the predetermined area of skin. Those of skill in the art will recognize that other geometries are possible and fully within the scope of the present disclosure.

FIGS. 8L and 8M are a perspective view and a cross-sectional view, respectively, depicting an applicator 150 having a compressible distal end 1450. As shown in FIGS. 8L and 8M, applicator 150 can also include applicator housing 702, sheath 704 to which compressible distal end 1450 is attached, sharp 2502, and sensor 104.

According to some embodiments, in operation, the compressible distal end 1450 of applicator is first positioned on a skin surface of the subject. The subject then applies a force on the applicator, e.g., in a distal direction, which causes compressible distal end 1450 to stretch and flatten the portion of the skin surface beneath. In some embodiments, for example, compressible distal end 1450 can be comprised of an elastomeric material and biased in a radially inward direction. In other embodiments, compressible distal end 1450 can be biased in a radially outward direction. The force on the applicator can cause an edge portion of the compressible distal end 1450 in contact with the skin surface to be displaced in a radially outward direction, creating radially outward forces on the portion of the skin surface beneath the applicator, and causing the skin surface to be stretched and flattened.

Furthermore, according to some embodiments, applying the force on the applicator also causes a medical device, such as a sensor control unit, to advance from a first position within the applicator to a second position adjacent to the skin surface. According to one aspect of some embodiments, the compressible distal end 1450 can be in an unloaded state in the first position (e.g., before the force is applied on the applicator), and a loaded state in the second position (e.g., after the force is applied on the applicator). Subsequently, the medical device is applied to the stretched and flattened portion of the skin surface beneath the compressible distal end 1450. According to some embodiments, the application of the medical device can include placing an adhesive surface 105 of a sensor control unit 102 on the skin surface and/or positioning at least a portion of an analyte sensor under the skin surface. The analyte sensor can be an in vivo analyte sensor configured to measure an analyte level in a bodily fluid of the subject. In still other embodiments, the application of the medical device can include placing a drug-loaded patch on the skin surface. Those of skill in the art will appreciate that a compressible distal end can be utilized with any of the aforementioned medical applications and is not meant to be limited to use in an applicator for analyte sensor insertion.

### Example Embodiments of Sensor Carriers

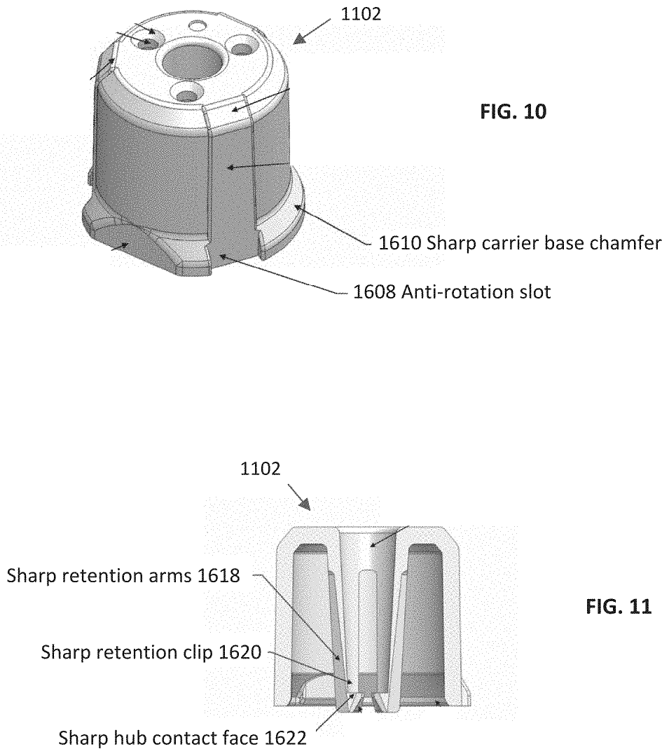

FIG. 9A is a proximal perspective view depicting an example embodiment of sensor carrier 710 that can retain sensor electronics within applicator 150. It can also retain sharp carrier 1102 with sharp module 2500. In this example embodiment, sensor carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of sensor carrier 710 extending outward and can lock sensor carrier 710 for added safety prior to firing. Rotation limiter 1506 can be a proximally extending relatively short protrusion on a proximal surface of sensor carrier 710 which limits rotation of carrier 710. Sharp carrier lock arms 1524 can interface with sharp carrier 1102 as described with reference to FIGS. 10 and 11 below.

FIG. 9B is a distal perspective view of sensor carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

### Example Embodiments of Sharp Carriers

FIGS. 10 and 11 are a proximal perspective view and a side cross-sectional view, respectively, depicting an example embodiment of sharp carrier 1102. Sharp carrier 1102 can grasp and retain sharp module 2500 within applicator 150. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

As shown in FIG. 11, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 17A).

### Example Embodiments of Sensor Modules

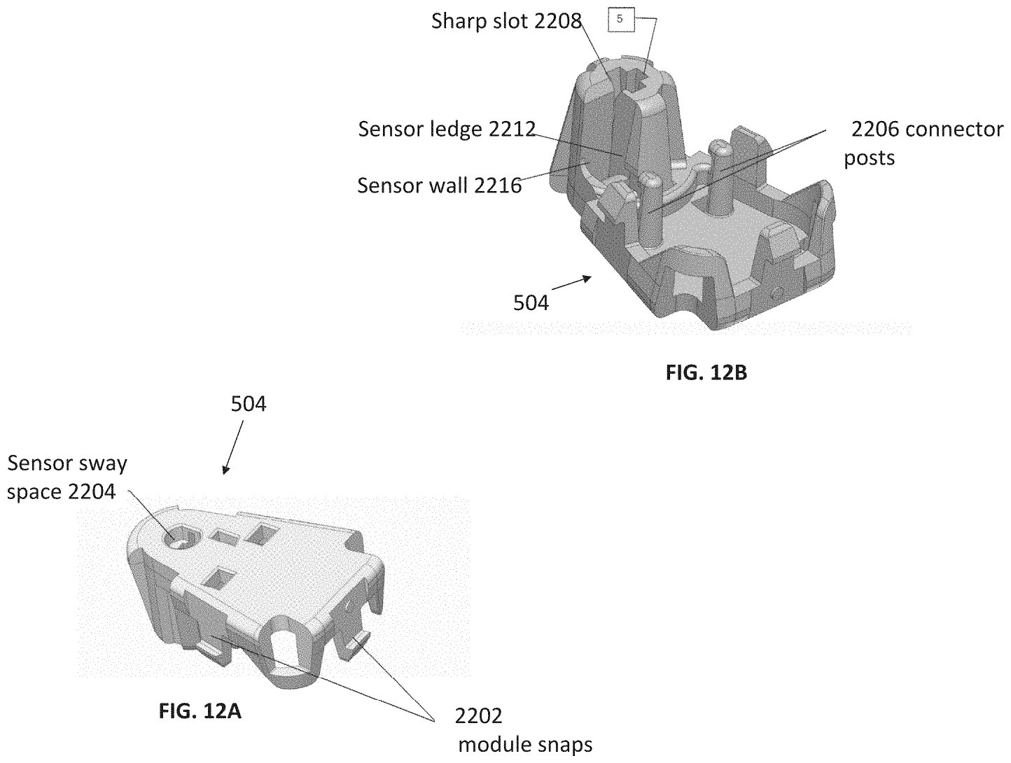

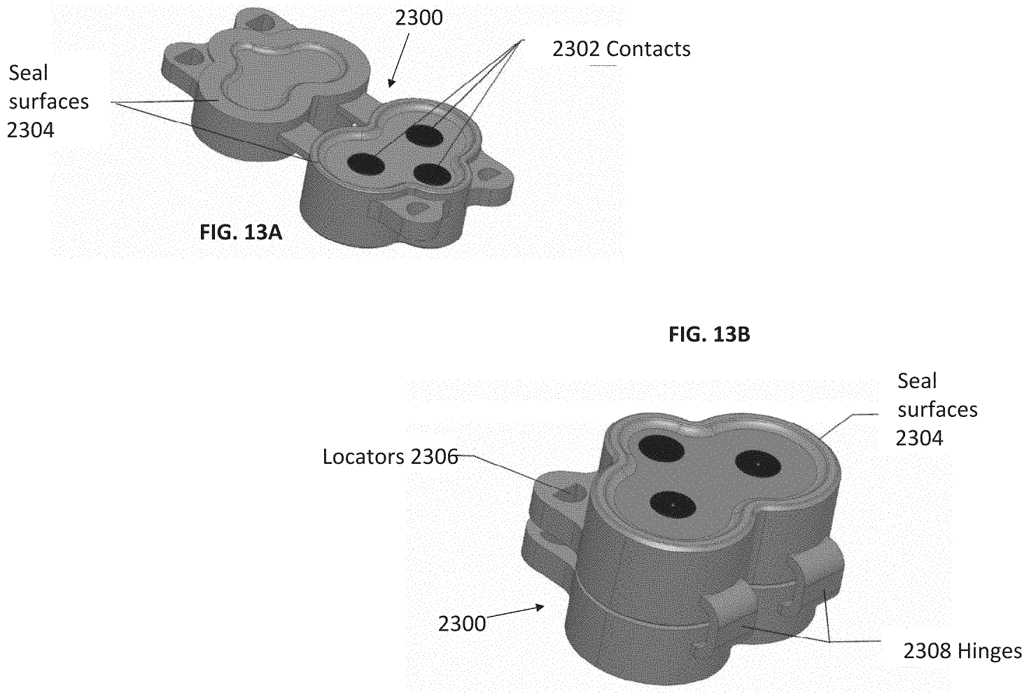

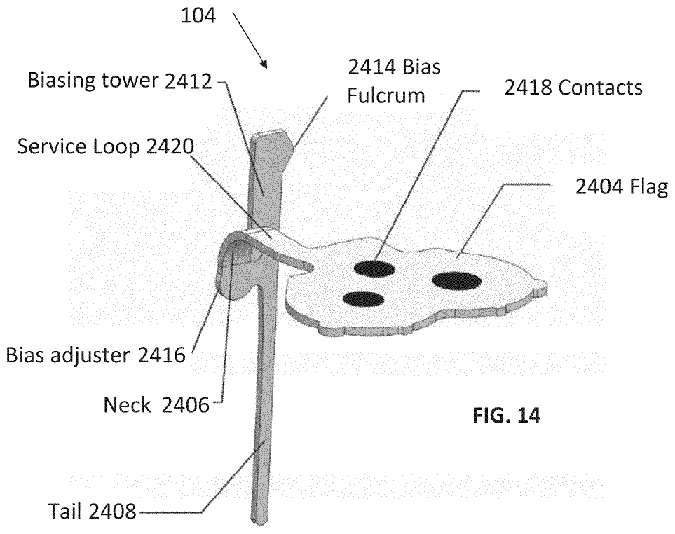

FIGS. 12A and 12B are a top perspective view and a bottom perspective view, respectively, depicting an example embodiment of sensor module 504. Module 504 can hold a connector 2300 (FIGS. 13A and 13B) and a sensor 104 (FIG. 14). Module 504 is capable of being securely coupled with electronics housing 706. One or more deflectable arms or module snaps 2202 can snap into the corresponding features 2010 of housing 706. A sharp slot 2208 can provide a location for sharp tip 2502 to pass through and sharp shaft 2504 to temporarily reside. A sensor ledge 2212 can define a sensor position in a horizontal plane, prevent a sensor from lifting connector 2300 off of posts and maintain sensor 104 parallel to a plane of connector seals. It can also define sensor bend geometry and minimum bend radius. It can limit sensor travel in a vertical direction and prevent a tower from protruding above an electronics housing surface and define a sensor tail length below a patch surface. A sensor wall 2216 can constrain a sensor and define a sensor bend geometry and minimum bend radius.

FIGS. 13A and 13B are perspective views depicting an example embodiment of connector 2300 in an open state and a closed state, respectively. Connector 2300 can be made of silicone rubber that encapsulates compliant carbon impregnated polymer modules that serve as electrical conductive contacts 2302 between sensor 104 and electrical circuitry contacts for the electronics within housing 706. The connector can also serve as a moisture barrier for sensor 104 when assembled in a compressed state after transfer from a container to an applicator and after application to a user's skin. A plurality of seal surfaces 2304 can provide a watertight seal for electrical contacts and sensor contacts. One or more hinges 2208 can connect two distal and proximal portions of connector 2300.

FIG. 14 is a perspective view depicting an example embodiment of sensor 104. A neck 2406 can be a zone which allows folding of the sensor, for example ninety degrees. A membrane on tail 2408 can cover an active analyte sensing element of the sensor 104. Tail 2408 can be the portion of sensor 104 that resides under a user's skin after insertion. A flag 2404 can contain contacts and a sealing surface. A biasing tower 2412 can be a tab that biases the tail 2408 into sharp slot 2208. A bias fulcrum 2414 can be an offshoot of biasing tower 2412 that contacts an inner surface of a needle to bias a tail into a slot. A bias adjuster 2416 can reduce a localized bending of a tail connection and prevent sensor trace damage. Contacts 2418 can electrically couple the active portion of the sensor to connector 2300. A service loop 2420 can translate an electrical path from a vertical direction ninety degrees and engage with sensor ledge 2212 (FIG. 12B).

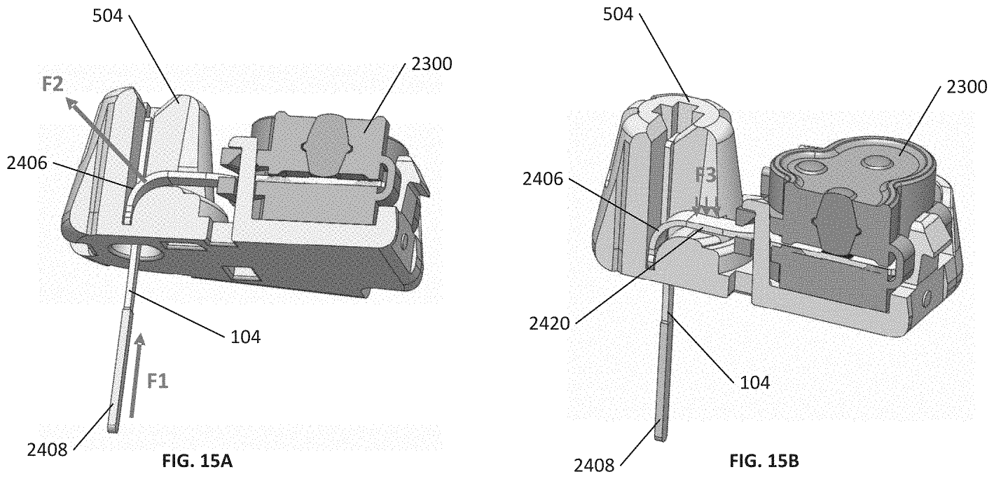

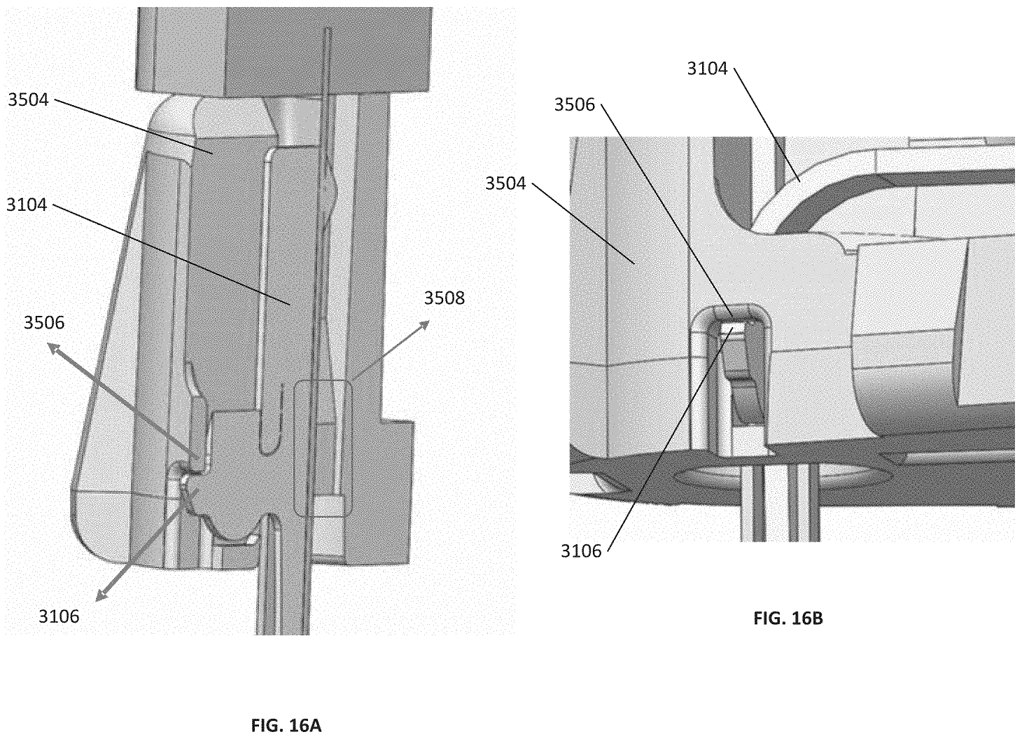

FIGS. 15A and 15B are bottom and top perspective views, respectively, depicting an example embodiment of a sensor module assembly comprising sensor module 504, connector 2300, and sensor 104. According to one aspect of the aforementioned embodiments, during or after insertion, sensor 104 can be subject to axial forces pushing up in a proximal direction against sensor 104 and into the sensor module 105, as shown by force, Fl, of FIG. 15A. According to some embodiments, this can result in an adverse force, F2, being applied to neck 2406 of sensor 104 and, consequently, result in adverse forces, F3, being translated to service loop 2420 of sensor 104. In some embodiments, for example, axial forces, Fl, can occur as a result of a sensor insertion mechanism in which the sensor is designed to push itself through the tissue, a sharp retraction mechanism during insertion, or due to a physiological reaction created by tissue surrounding sensor 104 (e.g., after insertion).

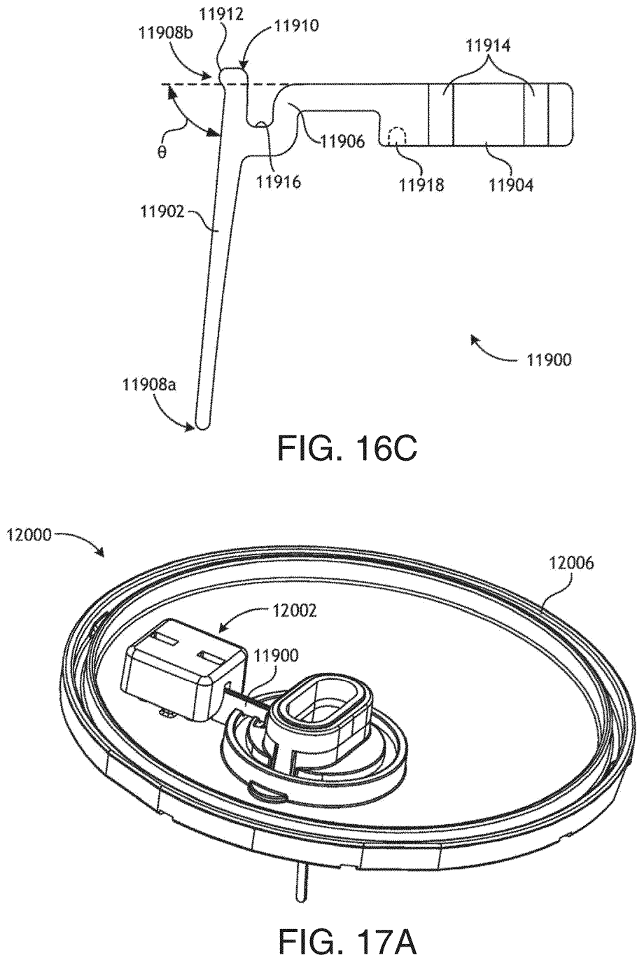

FIGS. 16A and 16B are close-up partial views of an example embodiment of a sensor module assembly having certain axial stiffening features. In a general sense, the embodiments described herein are directed to mitigating the effects of axial forces on the sensor as a result of insertion and/or retraction mechanisms, or from a physiological reaction to the sensor in the body. As seen in FIGS. 16A and 16B, according to one aspect of the embodiments, sensor 3104 comprises a proximal portion having a hook feature 3106 configured to engage a catch feature 3506 of the sensor module 3504. In some embodiments, sensor module 3504 can also include a clearance area 3508 to allow a distal portion of sensor 3104 to swing backwards during assembly to allow for the assembly of the hook feature 3106 of sensor 3104 over and into the catch feature 3506 of sensor module 3504.

According to another aspect of the embodiments, the hook and catch features 3106, 3506 operate in the following manner. Sensor 3104 includes a proximal sensor portion, coupled to sensor module 3504, as described above, and a distal sensor portion that is positioned beneath a skin surface in contact with a bodily fluid. As seen in FIGS. 16A and 16B, the proximal sensor portion includes a hook feature 3106 adjacent to the catch feature 3506 of sensor module 3504. During or after sensor insertion, one or more forces are exerted in a proximal direction along a longitudinal axis of sensor 3104. In response to the one or more forces, hook feature 3106 engages catch feature 3506 to prevent displacement of sensor 3104 in a proximal direction along the longitudinal axis.

According to another aspect of the embodiments, sensor 3104 can be assembled with sensor module 3504 in the following manner. Sensor 3104 is loaded into sensor module 3504 by displacing the proximal sensor portion in a lateral direction to bring the hook feature 3106 in proximity to the catch feature 3506 of sensor module 3504. More specifically, displacing the proximal sensor portion in a lateral direction causes the proximal sensor portion to move into clearance area 3508 of sensor module 3504.

Although FIGS. 16A and 16B depict hook feature 3106 as a part of sensor 3104, and catch feature 3506 as a part of sensor module 3504, those of skill in the art will appreciate that hook feature 3106 can instead be a part of sensor module 3504, and, likewise, catch feature 3506 can instead be a part of sensor 3106. Similarly, those of skill in the art will also recognize that other mechanisms (e.g., detent, latch, fastener, screw, etc.) implemented on sensor 3104 and sensor module 3504 to prevent axial displacement of sensor 3104 are possible and within the scope of the present disclosure.

FIG. 16C is a side view of an example sensor 11900, according to one or more embodiments of the disclosure. The sensor 11900 may be similar in some respects to any of the sensors described herein and, therefore, may be used in an analyte monitoring system to detect specific analyte concentrations. As illustrated, the sensor 11900 includes a tail 11902, a flag 11904, and a neck 11906 that interconnects the tail 11902 and the flag 11904. The tail 11902 includes an enzyme or other chemistry or biologic and, in some embodiments, a membrane may cover the chemistry. In use, the tail 11902 is transcutaneously received beneath a user's skin, and the chemistry included thereon helps facilitate analyte monitoring in the presence of bodily fluids.

The tail 11902 may be received within a hollow or recessed portion of a sharp (not shown) to at least partially circumscribe the tail 11902 of the sensor 11900. As illustrated, the tail 11902 may extend at an angle Q offset from horizontal. In some embodiments, the angle Q may be about 85°. Accordingly, in contrast to other sensor tails, the tail 11902 may not extend perpendicularly from the flag 11904, but instead at an angle offset from perpendicular. This may prove advantageous in helping maintain the tail 11902 within the keep the recessed portion of the sharp.

The tail 11902 includes a first or bottom end 11908a and a second or top end 11908b opposite the top end 11908a. A tower 11910 may be provided at or near the top end 11908b and may extend vertically upward from the location where the neck 11906 interconnects the tail 11902 to the flag 11904. During operation, if the sharp moves laterally, the tower 11910 will help picot the tail 11902 toward the sharp and otherwise stay within the recessed portion of the sharp. Moreover, in some embodiments, the tower 11910 may provide or otherwise define a protrusion 11912 that extends laterally therefrom. When the sensor 11900 is mated with the sharp and the tail 11902 extends within the recessed portion of the sharp, the protrusion 11912 may engage the inner surface of the recessed portion. In operation, the protrusion 11912 may help keep the tail 11902 within the recessed portion.

The flag 11904 may comprise a generally planar surface having one or more sensor contacts 11914 arranged thereon. The sensor contact(s) 11914 may be configured to align with a corresponding number of compliant carbon impregnated polymer modules encapsulated within a connector.

In some embodiments, as illustrated, the neck 11906 may provide or otherwise define a dip or bend 11916 extending between the flag 11904 and the tail 11902. The bend 11916 may prove advantageous in adding flexibility to the sensor 11900 and helping prevent bending of the neck 11906.

In some embodiments, a notch 11918 (shown in dashed lines) may optionally be defined in the flag near the neck 11906. The notch 11918 may add flexibility and tolerance to the sensor 11900 as the sensor 11900 is mounted to the mount. More specifically, the notch 11918 may help take up interference forces that may occur as the sensor 11900 is mounted within the mount.

FIGS. 17A and 17B are isometric and partially exploded isometric views of an example connector assembly 12000, according to one or more embodiments. As illustrated, the connector assembly 12000 may include a connector 12002, and FIG. 17C is an isometric bottom view of the connector 12002. The connector 12002 may comprise an injection molded part used to help secure one or more compliant carbon impregnated polymer modules 12004 (four shown in FIG. 17B) to a mount 12006. More specifically, the connector 12002 may help secure the modules 12004 in place adjacent the sensor 11900 and in contact with the sensor contacts 11914 (FIG. 16C) provided on the flag 11904 (FIG. 16C). The modules 12004 may be made of a conductive material to provide conductive communication between the sensor 11900 and corresponding circuitry contacts (not shown) provided within the mount 12006.

As best seen in FIG. 17C, the connector 12002 may define pockets 12008 sized to receive the modules 12004. Moreover, in some embodiments, the connector 12002 may further define one or more depressions 12010 configured to mate with one or more corresponding flanges 12012 (FIG. 17B) on the mount 12006. Mating the depressions 12010 with the flanges 12012 may secure the connector 12002 to the mount 12006 via an interference fit or the like. In other embodiments, the connector 12002 may be secured to the mount 12006 using an adhesive or via sonic welding.

FIGS. 17D and 17E are isometric and partially exploded isometric views of another example connector assembly 12100, according to one or more embodiments. As illustrated, the connector assembly 12100 may include a connector 12102, and FIG. 17F is an isometric bottom view of the connector 12102. The connector 12102 may comprise an injection molded part used to help keep one or more compliant metal contacts 12104 (four shown in FIG. 17E) secured against the sensor 11900 on a mount 12106. More specifically, the connector 12102 may help secure the contacts 12104 in place adjacent the sensor 11900 and in contact with the sensor contacts 11914 (FIG. 16C) provided on the flag 11904. The contacts 12104 may be made of a stamped conductive material that provides conductive communication between the sensor 11900 and corresponding circuitry contacts (not shown) provided within the mount 12106. In some embodiments, for example, the contacts 12104 may be soldered to a PCB (not shown) arranged within the mount 12106.

As best seen in FIG. 17F, the connector 12102 may define pockets 12108 sized to receive the contacts 12104. Moreover, in some embodiments, the connector 12102 may further define one or more depressions 12110 configured to mate with one or more corresponding flanges 12112 (FIG. 120B) on the mount 12006. Mating the depressions 12110 with the flanges 12112 may help secure the connector 12102 to the mount 12106 via an interference fit or the like. In other embodiments, the connector 12102 may be secured to the mount 12106 using an adhesive or via sonic welding.

### Example Embodiments of Sharp Modules

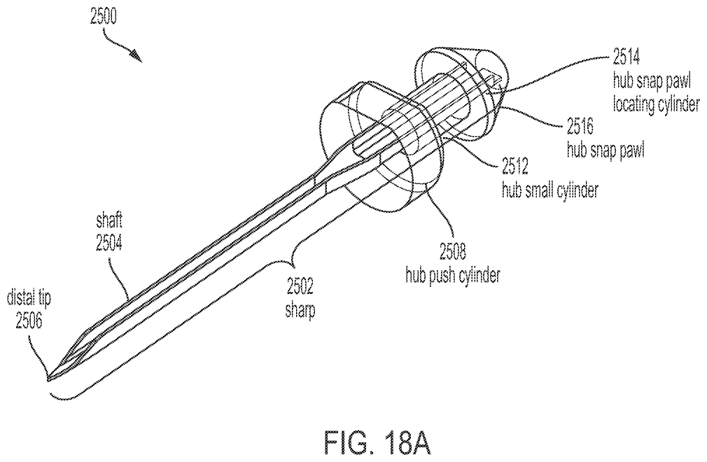

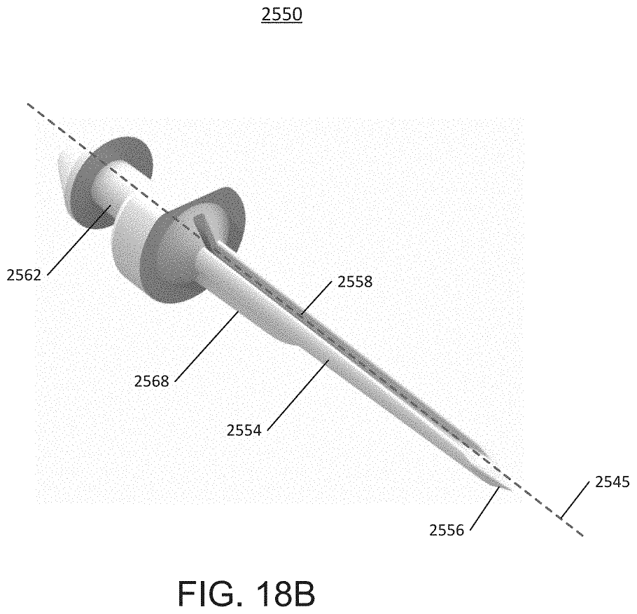

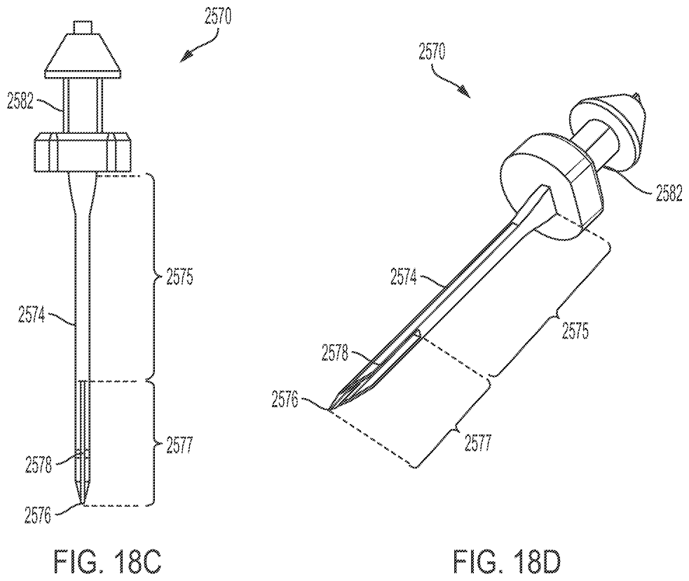

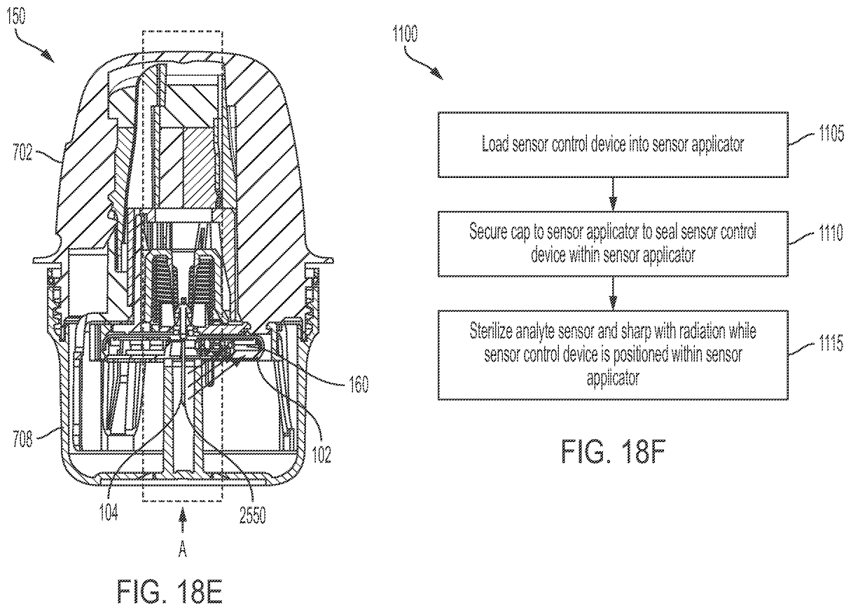

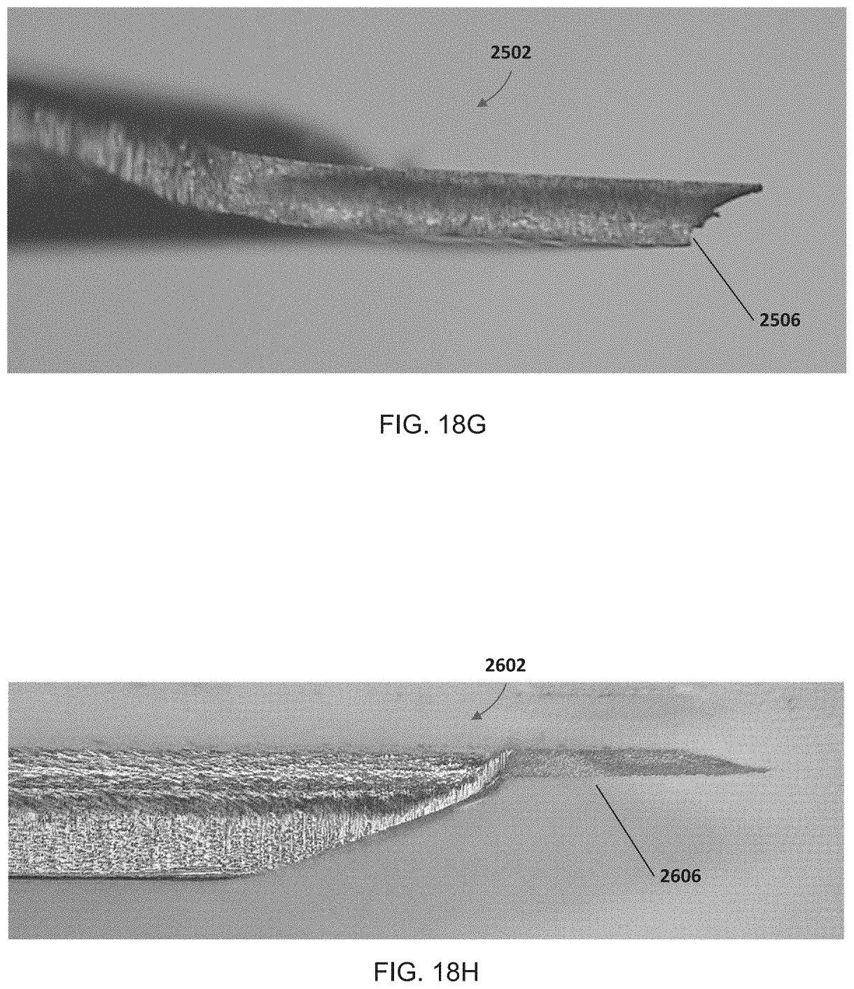

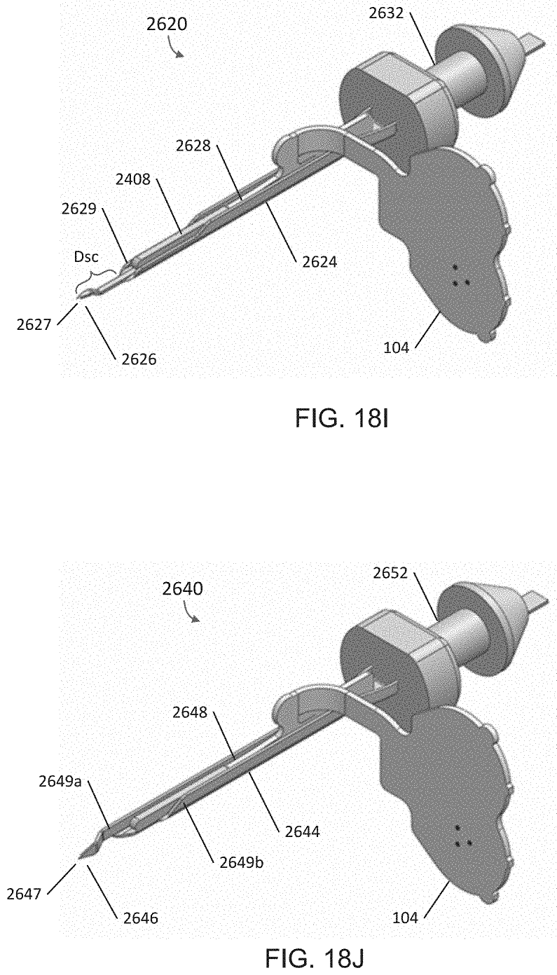

FIG. 18A is a perspective view depicting an example embodiment of sharp module 2500 prior to assembly within sensor module 504 (FIG. 6B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 11). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500. Further details regarding embodiments of sharp modules, sharps, their components, and variants thereof, are described in U.S. Patent Publication No. 2014/0171771, which is incorporated by reference herein in its entirety and for all purposes.

FIGS. 18B, 18C, and 18D depict example embodiments of plastic sharp modules. By way of background, according to one aspect of the embodiments, a plastic sharp can be advantageous in at least two respects.

First, relative to a metallic sharp, a plastic sharp can cause reduced trauma to tissue during the insertion process into the skin. Due to their manufacturing process, e.g., chemical etching and mechanical forming, metallic sharps are typically characterized by sharp edges and burrs that can cause trauma to tissue at the insertion site. By contrast, a plastic sharp can be designed to have rounded edges and a smooth finish to reduce trauma as the sharp is positioned through tissue. Moreover, those of skill in the art will understand that reducing trauma during the insertion process can lead to reduced ESA and improve accuracy in analyte level readings soon after insertion.

Second, a plastic sharp can simplify the applicator manufacturing and assembly process. As with earlier described embodiments, certain applicators are provided to the user in two pieces: (1) an applicator containing the sharp and sensor electronics in a sensor control unit, and (2) a sensor container. This requires the user to assemble the sensor into the sensor control unit. One reason for a two-piece assembly is to allow for electron beam sterilization of the sensor to occur separately from the applicator containing the metallic sharp and the sensor electronics. Metallic sharps, e.g., sharps made of stainless steel, have a higher density relative to sharps made of polymeric or plastic materials. As a result, electron beam scatter from an electron beam striking a metallic sharp can damage the sensor electronics of the sensor control unit. By utilizing a plastic sharp, e.g., a sharp made of polymeric materials, and additional shielding features to keep the electron beam path away from the sensor electronics, the applicator and sensor can be sterilized and packaged in a single package, thereby reducing the cost to manufacture and simplifying the assembly process for the user.

Referring to FIG. 18B, a perspective view of an example embodiment of plastic sharp module 2550 is shown, and can include a hub 2562 coupled to a proximal end of the sharp, sharp shaft 2554, a sharp distal tip 2556 configured to penetrate a skin surface, and a sensor channel 2558 configured to receive at least a portion of an analyte sensor 104. Any or all of the components of sharp module 2550 can be comprised of a plastic material such as, for example, a thermoplastic material, a liquid crystal polymer (LCP), or a similar polymeric material. According to some embodiments, for example, the sharp module can comprise a polyether ether ketone material. In other embodiments, silicone or other lubricants can be applied to an external surface of the sharp module and/or incorporated into the polymer material of the sharp module, to reduce trauma caused during the insertion process. Furthermore, to reduce trauma during insertion, one or more of sharp shaft 2554, sharp distal tip 2556, or alignment feature 2568 (described below) can include filleted and/or smoothed edges.

According to some embodiments, when assembled, the distal end of the analyte sensor can be in a proximal position relative to the sharp distal tip 2556. In other embodiments, the distal end of the analyte sensor and the sharp distal tip 2556 are co-localized.

According to another aspect of some embodiments, plastic sharp module 2550 can also include an alignment feature 2568 configured to prevent rotational movement along a vertical axis 2545 of sharp module 2550 during the insertion process, wherein the alignment feature 2568 can be positioned along a proximal portion of sharp shaft 2554.

FIGS. 18C and 18D are a side view and a perspective view, respectively, depicting another example embodiment of a plastic sharp module 2570. Like the embodiment described with respect to FIG. 18B, plastic sharp module 2570 can include a hub 2582 coupled to a proximal end of the sharp, a sharp shaft 2574, a sharp distal tip 2576 configured to penetrate a skin surface, and a sensor channel 2578 configured to receive at least a portion of an analyte sensor 104. Any or all of the components of sharp module 2570 can be comprised of a plastic material such as, for example, a thermoplastic material, LCP, or a similar polymeric material. In some embodiments, silicone or other lubricants can be applied to an external surface of sharp module 2570 and/or incorporated into the polymer material of sharp module 2570, to reduce trauma caused during the insertion process.

According to some embodiments, sharp shaft 2574 can include a distal portion 2577 that terminates at distal tip 2576, in which at least a portion of sensor channel 2578 is disposed. Sharp shaft 2574 can also have a proximal portion 2575 that is adjacent to distal portion 2577, wherein the proximal portion 2575 is solid, partially solid, or hollow, and is coupled to hub 2582. Although FIGS. 18C and 18D depict sensor channel 2578 as being located only within distal portion 2577, those of skill in the art will understand that sensor channel 2578 can also extend through a majority of, or along the entire length of, sharp shaft 2574 (e.g., as shown in FIG. 18B), including through at least a portion of proximal portion 2575. In addition, according to another aspect of some embodiments, at least a portion of proximal portion 2575 can have a wall thickness that is greater than the wall thickness of distal portion 2577, to reduce the possibility of stress buckling of the sharp during the insertion process. According to another aspect of some embodiments, plastic sharp module 2570 can include one or more ribs (not shown) adjacent to sharp hub portion 2582 to reduce the compressive load around hub 2582, and to mitigate stress buckling of the sharp during the insertion process.

FIG. 18E is a cross-sectional view depicting an example embodiment of an applicator 150 with a plastic sharp module during an electron beam sterilization process. As indicated by the rectangular area, A, an electron beam is focused on sensor 104 and plastic sharp 2550 of applicator 150 during a sterilization process. According to some embodiments, a cap 708 has been secured to applicator housing 702 to seal sensor control device 102 within applicator 150. During the sterilization process, electron beam scatter, as indicated by the diagonal arrows originating from plastic sharp 2550, in the direction and path of sensor electronics 160 has been reduced because a plastic sharp 2550 has been utilized instead of a metallic sharp. Although FIG. 18E depicts a focused electron beam sterilization process, those of skill in the art will recognize that an applicator with a plastic sharp module embodiment can also be utilized during a non-focused electron beam sterilization process.

FIG. 18F is a flow diagram depicting an example embodiment method 1100 for sterilizing an applicator assembly, according to the embodiments described above. At Step 1105, a sensor control device 102 is loaded into the applicator 150. Sensor control device 102 can include various components, including an electronics housing, a printed circuit board positioned within the electronics housing and containing processing circuitry, an analyte sensor extending from a bottom of the electronics housing, and a plastic sharp module having a plastic sharp that extends through the electronics housing. According to some embodiments, the plastic sharp can also receive the portion of the analyte sensor extending from the bottom of the electronics housing. As previously described, at Step 1110, a cap 708 is secured to the applicator housing 702 of applicator 150, thereby sealing the sensor control device 102 within applicator 150. At Step 1115, the analyte sensor 104 and plastic sharp 2550 are sterilized with radiation while sensor control device 102 is positioned within applicator 150.

According to some embodiments, sensor control device 102 can also include at least one shield positioned within the electronics housing, wherein the one or more shields are configured to shield the processing circuitry from radiation during the sterilization process. In some embodiments, the shield can comprise a magnet that generates a static magnetic field to divert radiation away from the processing circuitry. In this manner, the combination of the plastic sharp module and the magnetic shields/deflectors can operate in concert to protect the sensor electronics from radiation during the sterilization process.

Another example embodiment of a sharp designed to reduce trauma during a sensor insertion and retraction process will now be described. More specifically, certain embodiments described herein are directed to sharps comprising a metallic material (e.g., stainless steel) and manufactured through a coining process. According to one aspect of the embodiments, a coined sharp can be characterized as having a sharp tip with all other edges comprising rounded edges. As previously described, metallic sharps manufactured through a chemical etching and mechanical forming process can result in sharp edges and unintended hook features. For example, FIG. 18G is a photograph depicting a metallic sharp 2502 manufactured by a chemical etching and mechanical forming process. As seen in FIG. 18G, metallic sharp 2502 includes a sharp distal tip 2506 with a hook feature. These and other unintended transition features can result in increased trauma to tissue during a sensor insertion and retraction process. By contrast, FIG. 18H is a photograph depicting a coined sharp 2602, that is, a metallic sharp manufactured through a coining process. As seen in FIG. 18H, coined sharp 2602 also includes a sharp distal tip 2606. Coined sharp 2602, however, includes only smooth, rounded edges without any unintended sharp edges or transitions.

As with previously described sharp embodiments, the coined sharp 2602 embodiments described herein can also be assembled into a sharp module having a sharp portion and a hub portion. Likewise, the sharp portion comprises a sharp shaft, a sharp proximal end coupled to a distal end of the hub portion, and a sharp distal tip configured to penetrate a skin surface. According to one aspect of the embodiments, one or all of the sharp portion, the sharp shaft, and/or the sharp distal tip of a coined sharp 2602 can comprise one or more rounded edges.

Furthermore, it will be understood by those of skill in the art that the coined sharp 2602 embodiments described herein can similarly be used with any of the sensors described herein, including in vivo analyte sensors that are configured to measure an analyte level in a bodily fluid of a subject. For example, in some embodiments, coined sharp 2602 can include a sensor channel (not shown) configured to receive at least a portion of an analyte sensor. Likewise, in some embodiments of the sharp module assembly utilizing a coined sharp 2602, the distal end of the analyte sensor can be in a proximal position relative to the sharp distal tip 2606. In other embodiments, the distal end of the analyte sensor and the sharp distal tip 2606 are co-localized.

Other example embodiments of sharps designed to reduce trauma during a sensor insertion process will now be described. Referring back to FIG. 18A, an example embodiment of sharp module 2500 (shown without analyte sensor) is depicted, and includes a sharp 2502 comprising a sensor channel having a U-shaped geometry configured to receive at least a portion of an analyte sensor, and a distal tip 2506 configured to penetrate a skin surface during the sensor insertion process.

In certain embodiments, sharp module can include a sharp having a distal tip with an offset geometry configured to create a smaller opening in the skin relative to other sharps (e.g., sharp 2502 depicted in FIG. 18A). Turning to FIG. 18I, a perspective view of an example embodiment of a sharp module 2620 (with analyte sensor 104) having an offset tip portion is shown. Similar to the previously described sharp modules, sharp module 2620 can include a sharp shaft 2624 coupled to hub 2632 at a proximal end, sensor channel 2628 configured to receive at least a portion of analyte sensor 104, and a distal tip 2626 configured to penetrate a skin surface during the sensor insertion process.

According to one aspect of the embodiment, one or more sidewalls 2629 that form sensor channel 2628 are disposed along sharp shaft 2624 at a predetermined distance, Dsc, from distal tip 2626. In certain embodiments, predetermined distance, Dsc, can be between 1 mm and 8 mm. In other embodiments, predetermined distance, Dsc, can be between 2 mm and 5 mm. Those of skill in the art will recognize that other predetermined distances, Dsc, can be utilized and are fully within the scope of the present disclosure. In other words, according to some embodiments, sensor channel 2628 is in a spaced relation to distal tip 2626. In this regard, distal tip 2626 has a reduced cross-sectional footprint relative to, for example, distal tip 2506 of sharp module 2500, whose sensor channel is adjacent to distal tip 2506. According to another aspect of the embodiment, at the terminus of distal tip 2626 is an offset tip portion 2627 configured to prevent sensor tip 2408 from being damaged during insertion and to create a small opening in the skin. In some embodiments, offset tip portion 2627 can be a separate element coupled to a distal end of sharp shaft 2624. In other embodiments, offset tip portion 2627 can be formed from a portion of distal tip 2506 or sharp shaft 2624. During insertion, as the sharp moves into the skin surface, offset tip portion 2627 can cause the skin surrounding the skin opening to stretch and widen in a lateral direction without further cutting of skin tissue. In this regard, less trauma results during the sensor insertion process.

Referring next to FIG. 18J, a perspective view of another example embodiment of a sharp module 2640 (with analyte sensor 104) having an offset tip portion is shown. Like the previous embodiments, sharp module 2640 can include a sharp shaft 2644 coupled to hub 2652 at a proximal end, sensor channel 2648 configured to receive at least a portion of analyte sensor 104, and a distal tip 2646 configured to penetrate a skin surface during the sensor insertion process. According to one aspect of the embodiment, sensor channel 2648 can comprise a first sidewall 2649a and a second sidewall 2649b, wherein first sidewall 2649a extends to the distal tip 2646, wherein a terminus of first sidewall 2649a forms the offset tip portion 2647, and wherein second sidewall 2649b is disposed along sharp shaft 2644 at a predetermined distance from distal tip 2646, and wherein a terminus of second sidewall 2649b is proximal to the terminus of first sidewall 2649a. Those of skill in the art will appreciate that in other embodiments, second sidewall 2649b can extend to the distal tip 2646 to form the offset tip portion 2647, instead of first sidewall 2649a. In addition, offset tip portion 2647 can be formed from a third or fourth sidewall (not shown), and such geometries are fully within the scope of the present disclosure.

With respect to the sharp and sharp module embodiments described herein, those of skill in the art will recognize that any or all of the components can comprise either a metallic material, such as stainless steel, or a plastic material, such as a liquid crystal polymer. Furthermore, it will be understood by those of skill in the art that any of the sharp and/or sharp module embodiments described herein can be used or combined with any of the sensors, sensor modules, sensor carriers, sheaths, applicator devices, or any of the other analyte monitoring system components described herein.

### Example Embodiments of Applicators and Sensor Control Devices for One Piece Architectures

Referring briefly again to FIGS. 1 and 3A-3G, for the two-piece architecture system, the sensor tray 202 and the sensor applicator 102 are provided to the user as separate packages, thus requiring the user to open each package and finally assemble the system. In some applications, the discrete, sealed packages allow the sensor tray 202 and the sensor applicator 102 to be sterilized in separate sterilization processes unique to the contents of each package and otherwise incompatible with the contents of the other. More specifically, the sensor tray 202, which includes the plug assembly 207, including the sensor 110 and the sharp 220, may be sterilized using radiation sterilization, such as electron beam (or “e-beam”) irradiation. Radiation sterilization, however, can damage the electrical components arranged within the electronics housing of the sensor control device 102. Consequently, if the sensor applicator 102, which contains the electronics housing of the sensor control device 102, needs to be sterilized, it may be sterilized via another method, such as gaseous chemical sterilization using, for example, ethylene oxide. Gaseous chemical sterilization, however, can damage the enzymes or other chemistry and biologies included on the sensor 110. Because of this sterilization incompatibility, the sensor tray 202 and the sensor applicator 102 are commonly sterilized in separate sterilization processes and subsequently packaged separately, which requires the user to finally assemble the components for use.

According to embodiments of the present disclosure, the sensor control device 102 may be modified to provide a one-piece architecture that may be subjected to sterilization techniques specifically designed for a one-piece architecture sensor control device. A one-piece architecture allows the sensor applicator 150 and the sensor control device 102 to be shipped to the user in a single, sealed package that does not require any final user assembly steps. Rather, the user need only open one package and subsequently deliver the sensor control device 102 to the target monitoring location. The one-piece system architecture described herein may prove advantageous in eliminating component parts, various fabrication process steps, and user assembly steps. As a result, packaging and waste are reduced, and the potential for user error or contamination to the system is mitigated.

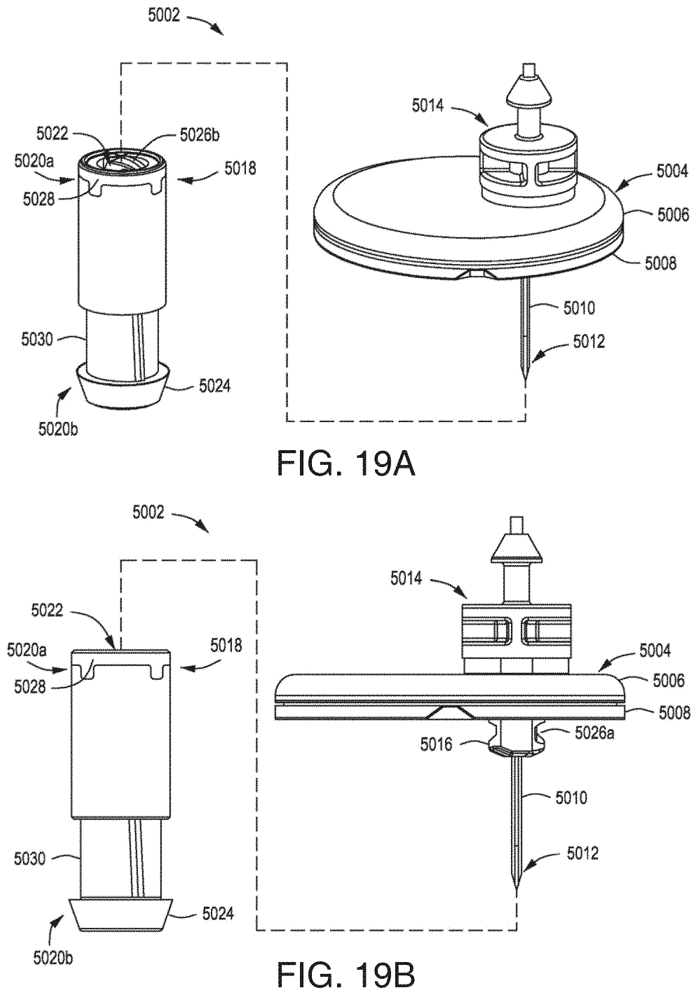

FIGS. 19A and 19B are isometric and side views, respectively, of another example sensor control device 5002, according to one or more embodiments of the present disclosure. The sensor control device 5002 may be similar in some respects to the sensor control device 102 of FIG. 1 and therefore may be best understood with reference thereto. Moreover, the sensor control device 5002 may replace the sensor control device 102 of FIG. 1 and, therefore, may be used in conjunction with the sensor applicator 102 of FIG. 1, which may deliver the sensor control device 5002 to a target monitoring location on a user's skin.

Unlike the sensor control device 102 of FIG. 1, however, the sensor control device 5002 may comprise a one-piece system architecture not requiring a user to open multiple packages and finally assemble the sensor control device 5002 prior to application. Rather, upon receipt by the user, the sensor control device 5002 may already be fully assembled and properly positioned within the sensor applicator 150 (FIG. 1). To use the sensor control device 5002, the user need only open one barrier (e.g., the applicator cap 708 of FIG. 3B) before promptly delivering the sensor control device 5002 to the target monitoring location for use.

As illustrated, the sensor control device 5002 includes an electronics housing 5004 that is generally disc-shaped and may have a circular cross-section. In other embodiments, however, the electronics housing 2004 may exhibit other cross-sectional shapes, such as ovoid or polygonal, without departing from the scope of the disclosure. The electronics housing 5004 may be configured to house or otherwise contain various electrical components used to operate the sensor control device 5002. In at least one embodiment, an adhesive patch (not shown) may be arranged at the bottom of the electronics housing 5004. The adhesive patch may be similar to the adhesive patch 105 of FIG. 1, and may thus help adhere the sensor control device 5002 to the user's skin for use.

As illustrated, the sensor control device 5002 includes an electronics housing 5004 that includes a shell 5006 and a mount 5008 that is mateable with the shell 5006. The shell 5006 may be secured to the mount 5008 via a variety of ways, such as a snap fit engagement, an interference fit, sonic welding, one or more mechanical fasteners (e.g., screws), a gasket, an adhesive, or any combination thereof. In some cases, the shell 5006 may be secured to the mount 5008 such that a sealed interface is generated therebetween.

The sensor control device 5002 may further include a sensor 5010 (partially visible) and a sharp 5012 (partially visible), used to help deliver the sensor 5010 transcutaneously under a user's skin during application of the sensor control device 5002. As illustrated, corresponding portions of the sensor 5010 and the sharp 5012 extend distally from the bottom of the electronics housing 5004 (e.g., the mount 5008). The sharp 5012 may include a sharp hub 5014 configured to secure and carry the sharp 5012. As best seen in FIG. 19B, the sharp hub 5014 may include or otherwise define a mating member 5016. To couple the sharp 5012 to the sensor control device 5002, the sharp 5012 may be advanced axially through the electronics housing 5004 until the sharp hub 5014 engages an upper surface of the shell 5006 and the mating member 5016 extends distally from the bottom of the mount 5008. As the sharp 5012 penetrates the electronics housing 5004, the exposed portion of the sensor 5010 may be received within a hollow or recessed (arcuate) portion of the sharp 5012. The remaining portion of the sensor 5010 is arranged within the interior of the electronics housing 5004.

The sensor control device 5002 may further include a sensor cap 5018, shown exploded or detached from the electronics housing 5004 in FIGS. 19A-19B. The sensor cap 5016 may be removably coupled to the sensor control device 5002 (e.g., the electronics housing 5004) at or near the bottom of the mount 5008. The sensor cap 5018 may help provide a sealed barrier that surrounds and protects the exposed portions of the sensor 5010 and the sharp 5012 from gaseous chemical sterilization. As illustrated, the sensor cap 5018 may comprise a generally cylindrical body having a first end 5020a and a second end 5020b opposite the first end 5020a. The first end 5020a may be open to provide access into an inner chamber 5022 defined within the body. In contrast, the second end 5020b may be closed and may provide or otherwise define an engagement feature 5024. As described herein, the engagement feature 5024 may help mate the sensor cap 5018 to the cap (e.g., the applicator cap 708 of FIG. 3B) of a sensor applicator (e.g., the sensor applicator 150 of FIGS. 1 and 3A-3G), and may help remove the sensor cap 5018 from the sensor control device 5002 upon removing the cap from the sensor applicator.

The sensor cap 5018 may be removably coupled to the electronics housing 5004 at or near the bottom of the mount 5008. More specifically, the sensor cap 5018 may be removably coupled to the mating member 5016, which extends distally from the bottom of the mount 5008. In at least one embodiment, for example, the mating member 5016 may define a set of external threads 5026a (FIG. 19B) mateable with a set of internal threads 5026b (FIG. 19A) defined by the sensor cap 5018. In some embodiments, the external and internal threads 5026a,b may comprise a flat thread design (e.g., lack of helical curvature), which may prove advantageous in molding the parts. Alternatively, the external and internal threads 5026a,b may comprise a helical threaded engagement. Accordingly, the sensor cap 5018 may be threadably coupled to the sensor control device 5002 at the mating member 5016 of the sharp hub 5014. In other embodiments, the sensor cap 5018 may be removably coupled to the mating member 5016 via other types of engagements including, but not limited to, an interference or friction fit, or a frangible member or substance that may be broken with minimal separation force (e.g., axial or rotational force).

In some embodiments, the sensor cap 5018 may comprise a monolithic (singular) structure extending between the first and second ends 5020a,b. In other embodiments, however, the sensor cap 5018 may comprise two or more component parts. In the illustrated embodiment, for example, the sensor cap 5018 may include a seal ring 5028 positioned at the first end 5020a and a desiccant cap 5030 arranged at the second end 5020b. The seal ring 5028 may be configured to help seal the inner chamber 5022, as described in more detail below. In at least one embodiment, the seal ring 5028 may comprise an elastomeric O-ring. The desiccant cap 5030 may house or comprise a desiccant to help maintain preferred humidity levels within the inner chamber 5022. The desiccant cap 5030 may also define or otherwise provide the engagement feature 5024 of the sensor cap 5018.

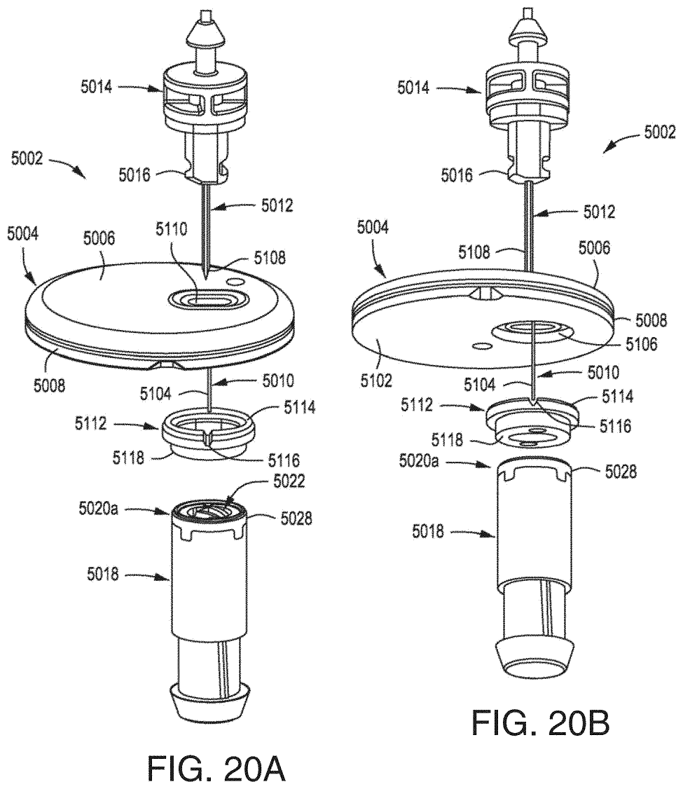

FIGS. 20A and 20B are exploded isometric top and bottom views, respectively, of the sensor control device 5002, according to one or more embodiments. The shell 5006 and the mount 5008 operate as opposing clamshell halves that enclose or otherwise substantially encapsulate various electronic components of the sensor control device 5002. More specifically, electronic components may include, but are not limited to, a printed circuit board (PCB), one or more resistors, transistors, capacitors, inductors, diodes, and switches. A data processing unit and a battery may be mounted to or otherwise interact with the PCB. The data processing unit may comprise, for example, an application specific integrated circuit (ASIC) configured to implement one or more functions or routines associated with operation of the sensor control device 5002. More specifically, the data processing unit may be configured to perform data processing functions, where such functions may include, but are not limited to, filtering and encoding of data signals, each of which corresponds to a sampled analyte level of the user. The data processing unit may also include or otherwise communicate with an antenna for communicating with the reader device 120 (FIG. 1). The battery may provide power to the sensor control device 5002 and, more particularly, to the electronic components of the PCB. While not shown, the sensor control device 5002 may also include an adhesive patch that may be applied to the bottom 5102 (FIG. 20B) of the mount 5008, and may help adhere the sensor control device 5002 to the user's skin for use.

The sensor control device 5002 may provide or otherwise include a sealed subassembly that includes, among other component parts, the shell 5006, the sensor 5010, the sharp 5012, and the sensor cap 5018. The sealed subassembly of the sensor control device 5002 may help isolate the sensor 5010 and the sharp 5012 within the inner chamber 5022 (FIG. 20A) of the sensor cap 5018 during a gaseous chemical sterilization process, which might otherwise adversely affect the chemistry provided on the sensor 5010.

The sensor 5010 may include a tail 5104 that extends out an aperture 5106 (FIG. 20B) defined in the mount 5008 to be transcutaneously received beneath a user's skin. The tail 5104 may have an enzyme or other chemistry included thereon to help facilitate analyte monitoring. The sharp 5012 may include a sharp tip 5108 extendable through an aperture 5110 (FIG. 51A) defined by the shell 5006, and the aperture 5110 may be coaxially aligned with the aperture 5106 of the mount 5008. As the sharp tip 5108 penetrates the electronics housing 5004, the tail 5104 of the sensor 5010 may be received within a hollow or recessed portion of the sharp tip 5108. The sharp tip 5108 may be configured to penetrate the skin while carrying the tail 5104 to put the active chemistry of the tail 5104 into contact with bodily fluids.

The sharp tip 5108 may be advanced through the electronics housing 5004 until the sharp hub 5014 engages an upper surface of the shell 5006 and the mating member 5016 extends out the aperture 5106 in the bottom 5102 of the mount 5008. In some embodiments, a seal member (not shown), such as an O-ring or seal ring, may interpose the sharp hub 5014 and the upper surface of the shell 5006 to help seal the interface between the two components. In some embodiments, the seal member may comprise a separate component part, but may alternatively form an integral part of the shell 5006, such as being a co-molded or overmolded component part.

The sealed subassembly may further include a collar 5112 that is positioned within the electronics housing 5004 and extends at least partially into the aperture 5106. The collar 5112 may be a generally annular structure that defines or otherwise provides an annular ridge 5114 on its top surface. In some embodiments, as illustrated, a groove 5116 may be defined in the annular ridge 5114 and may be configured to accommodate or otherwise receive a portion of the sensor 5010 extending laterally within the electronics housing 5004.

In assembling the sealed subassembly, a bottom 5118 of the collar 5112 may be exposed at the aperture 5106 and may sealingly engage the first end 5020a of the sensor cap 5018 and, more particularly, the seal ring 5028. In contrast, the annular ridge 5114 at the top of the collar 5112 may sealingly engage an inner surface (not shown) of the shell 5006. In at least one embodiment, a seal member (not shown) may interpose the annular ridge 5114 and the inner surface of the shell 5006 to form a sealed interface. In such embodiments, the seal member may also extend (flow) into the groove 5116 defined in the annular ridge 5114 and thereby seal about the sensor 5010 extending laterally within the electronics housing 5004. The seal member may comprise, for example, an adhesive, a gasket, or an ultrasonic weld, and may help isolate the enzymes and other chemistry included on the tail 5104.

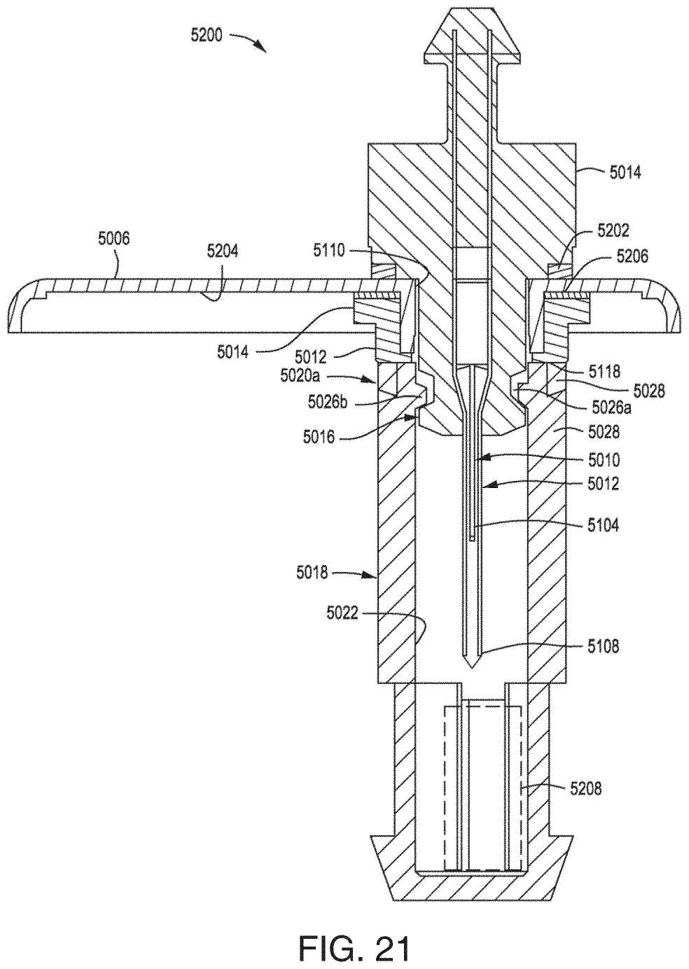

FIG. 21 is a cross-sectional side view of an assembled sealed subassembly 5200, according to one or more embodiments. The sealed subassembly 5200 may form part of the sensor control device 5002 of FIGS. 19A-19B and 20A-20B and may include portions of the shell 5006, the sensor 5010, the sharp 5012, the sensor cap 5018, and the collar 5112. The sealed subassembly 5200 may be assembled in a variety of ways. In one assembly process, the sharp 5012 may be coupled to the sensor control device 5002 by extending the sharp tip 5108 through the aperture 5110 defined in the top of the shell 5006 and advancing the sharp 5012 through the shell 5006 until the sharp hub 5014 engages the top of the shell 5006 and the mating member 196 extends distally from the shell 5006. In some embodiments, as mentioned above, a seal member 5202 (e.g., an O-ring or seal ring) may interpose the sharp hub 5014 and the upper surface of the shell 5006 to help seal the interface between the two components.

The collar 5112 may then be received over (about) the mating member 5016 and advanced toward an inner surface 5204 of the shell 5006 to enable the annular ridge 5114 to engage the inner surface 5204. A seal member 5206 may interpose the annular ridge 5114 and the inner surface 5204 and thereby form a sealed interface. The seal member 5206 may also extend (flow) into the groove 5116 (FIGS. 20A-20B) defined in the annular ridge 5114 and thereby seal about the sensor 5010 extending laterally within the electronics housing 5004 (FIGS. 20A-20B). In other embodiments, however, the collar 5112 may first be sealed to the inner surface 5204 of the shell 5006, following which the sharp 5012 and the sharp hub 5014 may be extended through the aperture 5110, as described above.

The sensor cap 5018 may be removably coupled to the sensor control device 5002 by threadably mating the internal threads 5026b of the sensor cap 5018 with the external threads 5026a of the mating member 5016. Tightening (rotating) the mated engagement between the sensor cap 5018 and the mating member 5016 may urge the first end 5020a of the sensor cap 5018 into sealed engagement with the bottom 5118 of the collar 5112. Moreover, tightening the mated engagement between the sensor cap 5018 and the mating member 5016 may also enhance the sealed interface between the sharp hub 5014 and the top of the shell 5006, and between the annular ridge 5114 and the inner surface 5204 of the shell 5006.

The inner chamber 5022 may be sized and otherwise configured to receive the tail 5104 and the sharp tip 5108. Moreover, the inner chamber 5022 may be sealed to isolate the tail 5104 and the sharp tip 5108 from substances that might adversely interact with the chemistry of the tail 5104. In some embodiments, a desiccant 5208 (shown in dashed lines) may be present within the inner chamber 5022 to maintain proper humidity levels.

Once properly assembled, the sealed subassembly 5200 may be subjected to any of the radiation sterilization processes mentioned herein to properly sterilize the sensor 5010 and the sharp 5012. This sterilization step may be undertaken apart from the remaining portions of the sensor control device (FIGS. 19A-19B and 20A-20B) to prevent damage to sensitive electrical components. The sealed subassembly 5200 may be subjected to radiation sterilization prior to or after coupling the sensor cap 5018 to the sharp hub 5014. When sterilized after coupling the sensor cap 5018 to the sharp hub 5014, the sensor cap 5018 may be made of a material that permits the propagation of radiation therethrough. In some embodiments, the sensor cap 5018 may be transparent or translucent, but can otherwise be opaque, without departing from the scope of the disclosure.

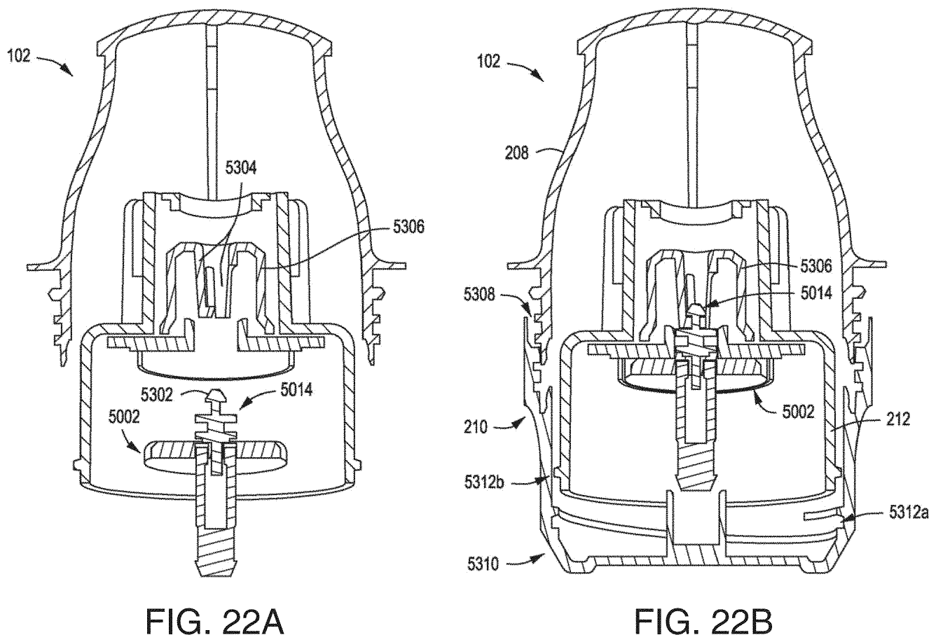

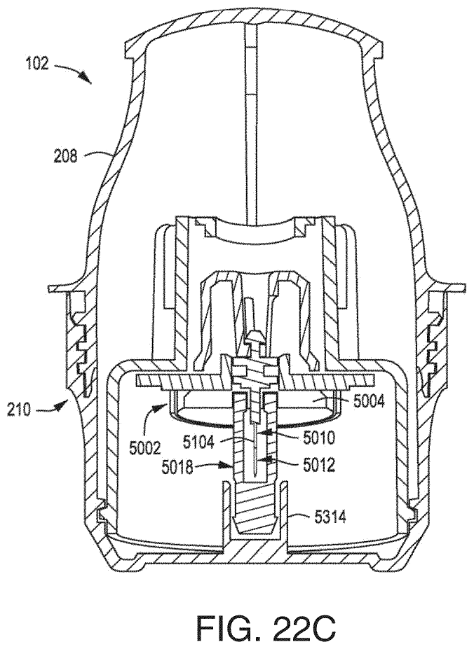

FIGS. 22A-22C are progressive cross-sectional side views showing assembly of the sensor applicator 102 with the sensor control device 5002, according to one or more embodiments. Once the sensor control device 5002 is fully assembled, it may then be loaded into the sensor applicator 102. With reference to FIG. 22A, the sharp hub 5014 may include or otherwise define a hub snap pawl 5302 configured to help couple the sensor control device 5002 to the sensor applicator 102. More specifically, the sensor control device 5002 may be advanced into the interior of the sensor applicator 102 and the hub snap pawl 5302 may be received by corresponding arms 5304 of a sharp carrier 5306 positioned within the sensor applicator 102.

In FIG. 22B, the sensor control device 5002 is shown received by the sharp carrier 5306 and, therefore, secured within the sensor applicator 102. Once the sensor control device 5002 is loaded into the sensor applicator 102, the applicator cap 210 may be coupled to the sensor applicator 102. In some embodiments, the applicator cap 210 and the housing 208 may have opposing, mateable sets of threads 5308 that enable the applicator cap 210 to be screwed onto the housing 208 in a clockwise (or counter-clockwise) direction and thereby secure the applicator cap 210 to the sensor applicator 102.

As illustrated, the sheath 212 is also positioned within the sensor applicator 102, and the sensor applicator 102 may include a sheath locking mechanism 5310 configured to ensure that the sheath 212 does not prematurely collapse during a shock event. In the illustrated embodiment, the sheath locking mechanism 5310 may comprise a threaded engagement between the applicator cap 210 and the sheath 212. More specifically, one or more internal threads 53 12a may be defined or otherwise provided on the inner surface of the applicator cap 210, and one or more external threads 53 12b may be defined or otherwise provided on the sheath 212. The internal and external threads 53 12a,b may be configured to threadably mate as the applicator cap 210 is threaded to the sensor applicator 102 at the threads 5308. The internal and external threads 53 12a,b may have the same thread pitch as the threads 5308 that enable the applicator cap 210 to be screwed onto the housing 208.

In FIG. 22C, the applicator cap 210 is shown fully threaded (coupled) to the housing 208. As illustrated, the applicator cap 210 may further provide and otherwise define a cap post 5314 centrally located within the interior of the applicator cap 210 and extending proximally from the bottom thereof. The cap post 5314 may be configured to receive at least a portion of the sensor cap 5018 as the applicator cap 210 is screwed onto the housing 208.

With the sensor control device 5002 loaded within the sensor applicator 102 and the applicator cap 210 properly secured, the sensor control device 5002 may then be subjected to a gaseous chemical sterilization configured to sterilize the electronics housing 5004 and any other exposed portions of the sensor control device 5002. Since the distal portions of the sensor 5010 and the sharp 5012 are sealed within the sensor cap 5018, the chemicals used during the gaseous chemical sterilization process are unable to interact with the enzymes, chemistry, and biologies provided on the tail 5104, and other sensor components, such as membrane coatings that regulate analyte influx.

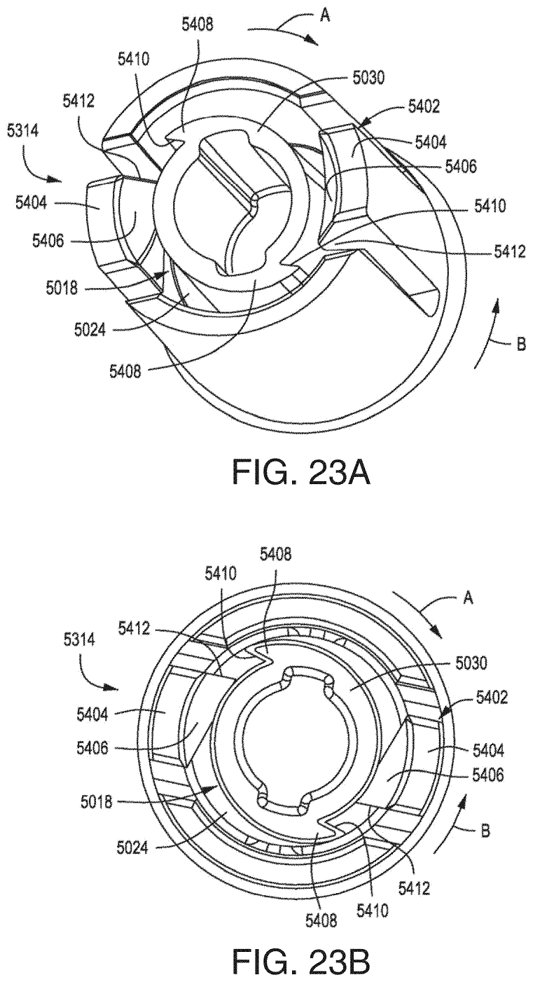

FIGS. 54A and 23B are perspective and top views, respectively, of the cap post 5314, according to one or more additional embodiments. In the illustrated depiction, a portion of the sensor cap 5018 is received within the cap post 5314 and, more specifically, the desiccant cap 5030 of the sensor cap 5018 is arranged within cap post 5314.

As illustrated, the cap post 5314 may define a receiver feature 5402 configured to receive the engagement feature 5024 of the sensor cap 5018 upon coupling (e.g., threading) the applicator cap 210 (FIG. 22C) to the sensor applicator 102 (FIGS. 22A-22C). Upon removing the applicator cap 210 from the sensor applicator 102, however, the receiver feature 5402 may prevent the engagement feature 914 from reversing direction and thus prevent the sensor cap 5018 from separating from the cap post 5314. Instead, removing the applicator cap 210 from the sensor applicator 102 will simultaneously detach the sensor cap 5018 from the sensor control device 5002 (FIGS. 19A-19B and 22A-22C), and thereby expose the distal portions of the sensor 5010 (FIGS. 22A-22C) and the sharp 5012 (FIGS. 22A-22C).

Many design variations of the receiver feature 5402 may be employed, without departing from the scope of the disclosure. In the illustrated embodiment, the receiver feature 5402 includes one or more compliant members 5404 (two shown) that are expandable or flexible to receive the engagement feature 5024 (FIGS. 19A-19B). The engagement feature 5024 may comprise, for example, an enlarged head and the compliant member(s) 5404 may comprise a collet-type device that includes a plurality of compliant fingers configured to flex radially outward to receive the enlarged head.

The compliant member(s) 5404 may further provide or otherwise define corresponding ramped surfaces 5406 configured to interact with one or more opposing camming surfaces 5408 provided on the outer wall of the engagement feature 5024. The configuration and alignment of the ramped surface(s) 5406 and the opposing camming surface(s) 5408 is such that the applicator cap 210 is able to rotate relative to the sensor cap 5018 in a first direction A (e.g., clockwise), but the cap post 5314 binds against the sensor cap 5018 when the applicator cap 210 is rotated in a second direction B (e.g., counter clockwise). More particularly, as the applicator cap 210 (and thus the cap post 5314) rotates in the first direction A, the camming surfaces 5408 engage the ramped surfaces 5406, which urge the compliant members 5404 to flex or otherwise deflect radially outward and results in a ratcheting effect. Rotating the applicator cap 210 (and thus the cap post 5314) in the second direction B, however, will drive angled surfaces 5410 of the camming surfaces 5408 into opposing angled surfaces 5412 of the ramped surfaces 5406, which results in the sensor cap 5018 binding against the compliant member(s) 5404.

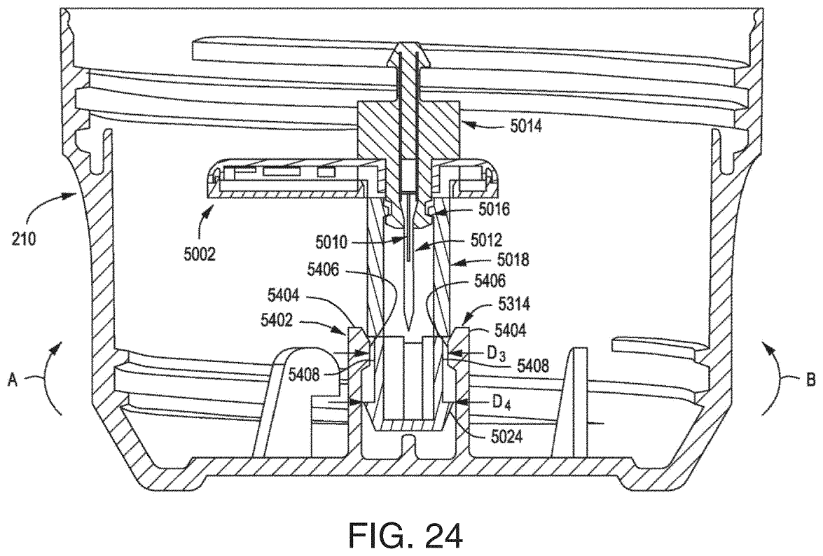

FIG. 24 is a cross-sectional side view of the sensor control device 5002 positioned within the applicator cap 210, according to one or more embodiments. As illustrated, the opening to the receiver feature 5402 exhibits a first diameter D3, while the engagement feature 5024 of the sensor cap 5018 exhibits a second diameter D4 that is larger than the first diameter D3 and greater than the outer diameter of the remaining portions of the sensor cap 5018. As the sensor cap 5018 is extended into the cap post 5314, the compliant member(s) 5404 of the receiver feature 5402 may flex (expand) radially outward to receive the engagement feature 5024. In some embodiments, as illustrated, the engagement feature 5024 may provide or otherwise define an angled or frustoconical outer surface that helps bias the compliant member(s) 5404 radially outward. Once the engagement feature 5024 bypasses the receiver feature 5402, the compliant member(s) 5404 are able to flex back to (or towards) their natural state and thus lock the sensor cap 5018 within the cap post 5314.

As the applicator cap 210 is threaded to (screwed onto) the housing 208 (FIGS. 22A-22C) in the first direction A, the cap post 5314 correspondingly rotates in the same direction and the sensor cap 5018 is progressively introduced into the cap post 5314. As the cap post 5314 rotates, the ramped surfaces 5406 of the compliant members 5404 ratchet against the opposing camming surfaces 5408 of the sensor cap 5018. This continues until the applicator cap 210 is fully threaded onto (screwed onto) the housing 208. In some embodiments, the ratcheting action may occur over two full revolutions of the applicator cap 210 before the applicator cap 210 reaches its final position.

To remove the applicator cap 210, the applicator cap 210 is rotated in the second direction B, which correspondingly rotates the cap post 5314 in the same direction and causes the camming surfaces 5408 (i.e., the angled surfaces 5410 of FIGS. 23A-23B) to bind against the ramped surfaces 5406 (i.e., the angled surfaces 5412 of FIGS. 23A-23B). Consequently, continued rotation of the applicator cap 210 in the second direction B causes the sensor cap 5018 to correspondingly rotate in the same direction and thereby unthread from the mating member 5016 to allow the sensor cap 5018 to detach from the sensor control device 5002. Detaching the sensor cap 5018 from the sensor control device 5002 exposes the distal portions of the sensor 5010 and the sharp 5012, and thus places the sensor control device 5002 in position for firing (use).

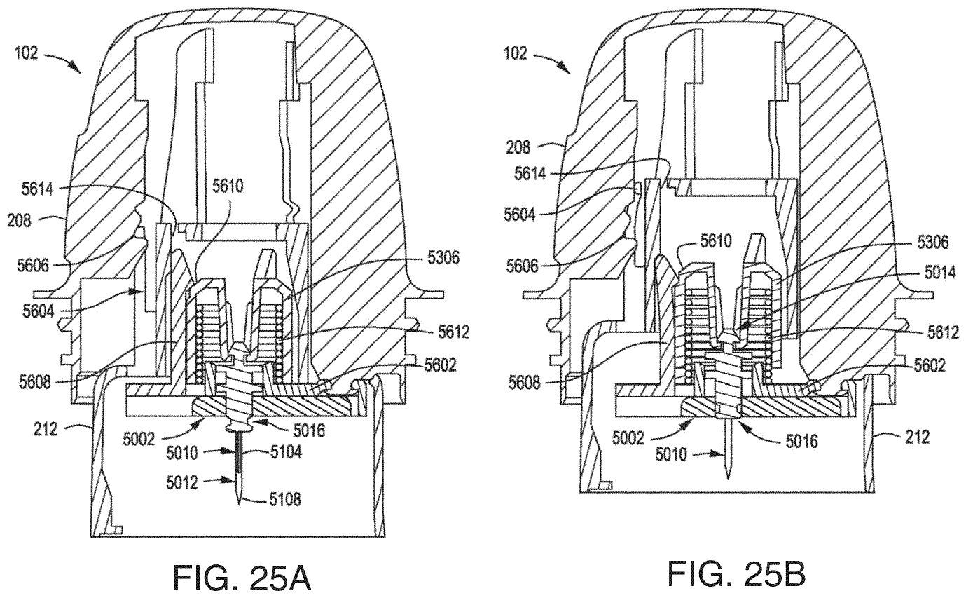

FIGS. 25A and 25B are cross-sectional side views of the sensor applicator 102 ready to deploy the sensor control device 5002 to a target monitoring location, according to one or more embodiments. More specifically, FIG. 25A depicts the sensor applicator 102 ready to deploy (fire) the sensor control device 5002, and FIG. 25B depicts the sensor applicator 102 in the process of deploying (firing) the sensor control device 5002. As illustrated, the applicator cap 210 (FIGS. 22A-22C and 55) has been removed, which correspondingly detaches (removes) the sensor cap 5018 (FIGS. 22A-22C and 55 and thereby exposes the tail 5104 of the sensor 5010 and the sharp tip 5108 of the sharp 5012, as described above. In conjunction with the sheath 212 and the sharp carrier 5306, the sensor applicator 102 also includes a sensor carrier 5602 (alternately referred to as a “puck” carrier) that helps position and secure the sensor control device 5002 within the sensor applicator 102.

Referring first to FIG. 25A, as illustrated, the sheath 212 includes one or more sheath arms 5604 (one shown) configured to interact with a corresponding one or more detents 5606 (one shown) defined within the interior of the housing 208. The detent(s) 5606 are alternately referred to as “firing” detent(s). When the sensor control device 5002 is initially installed in the sensor applicator 102, the sheath arms 5604 may be received within the detents 5606, which places the sensor applicator 102 in firing position. In the firing position, the mating member 5016 extends distally beyond the bottom of the sensor control device 5002. As discussed below, the process of firing the sensor applicator 102 causes the mating member 5016 to retract so that it does not contact the user's skin.

The sensor carrier 5602 may also include one or more carrier arms 5608 (one shown) configured to interact with a corresponding one or more grooves 5610 (one shown) defined on the sharp carrier 5306. A spring 5612 may be arranged within a cavity defined by the sharp carrier 5306 and may passively bias the sharp carrier 5306 upward within the housing 208. When the carrier arm(s) 5608 are properly received within the groove(s) 5610, however, the sharp carrier 5306 is maintained in position and prevented from moving upward. The carrier arm(s) 5608 interpose the sheath 212 and the sharp carrier 5306, and a radial shoulder 5614 defined on the sheath 212 may be sized to maintain the carrier arm(s) 5608 engaged within the groove(s) 5610 and thereby maintain the sharp carrier 5306 in position.

In FIG. 25B, the sensor applicator 102 is in the process of firing. As discussed herein with reference to FIGS. 3F-3G, this may be accomplished by advancing the sensor applicator 102 toward a target monitoring location until the sheath 212 engages the skin of the user. Continued pressure on the sensor applicator 102 against the skin may cause the sheath arm(s) 5604 to disengage from the corresponding detent(s) 5606, which allows the sheath 212 to collapse into the housing 208. As the sheath 212 starts to collapse, the radial shoulder 5614 eventually moves out of radial engagement with the carrier arm(s) 5608, which allows the carrier arm(s) 5608 to disengage from the groove(s) 5610. The passive spring force of the spring 5612 is then free to push upward on the sharp carrier 5306 and thereby force the carrier arm(s) 5608 out of engagement with the groove(s) 5610, which allows the sharp carrier 5306 to move slightly upward within the housing 208. In some embodiments, fewer coils may be incorporated into the design of the spring 5612 to increase the spring force necessary to overcome the engagement between carrier arm(s) 5608 and the groove(s) 5610. In at least one embodiment, one or both of the carrier arm(s) 5608 and the groove(s) 5610 may be angled to help ease disengagement.

As the sharp carrier 5306 moves upward within the housing 208, the sharp hub 5014 may correspondingly move in the same direction, which may cause partial retraction of the mating member 5016 such that it becomes flush, substantially flush, or sub-flush with the bottom of the sensor control device 5002. As will be appreciated, this ensures that the mating member 5016 does not come into contact with the user's skin, which might otherwise adversely impact sensor insertion, cause excessive pain, or prevent the adhesive patch (not shown) positioned on the bottom of the sensor control device 5002 from properly adhering to the skin.

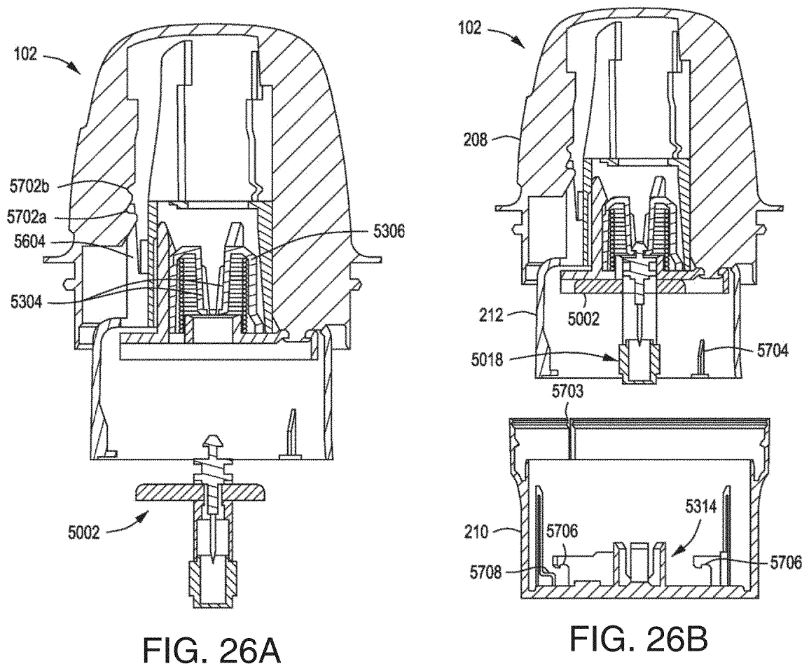

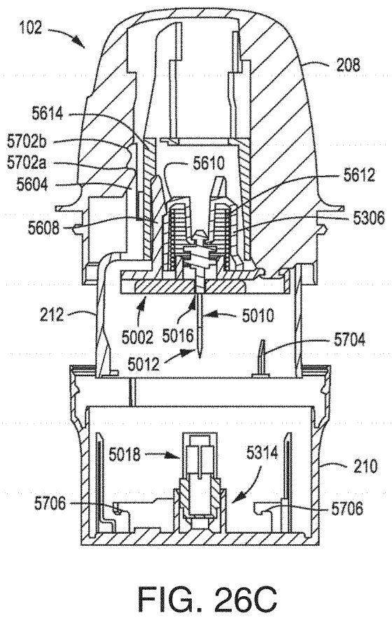

FIGS. 26A-26C are progressive cross-sectional side views showing assembly and disassembly of an alternative embodiment of the sensor applicator 102 with the sensor control device 5002, according to one or more additional embodiments. A fully assembled sensor control device 5002 may be loaded into the sensor applicator 102 by coupling the hub snap pawl 5302 into the arms 5304 of the sharp carrier 5306 positioned within the sensor applicator 102, as generally described above.

In the illustrated embodiment, the sheath arms 5604 of the sheath 212 may be configured to interact with a first detent 5702a and a second detent 5702b defined within the interior of the housing 208. The first detent 5702a may alternately be referred to a “locking” detent, and the second detent 5702b may alternately be referred to as a “firing” detent. When the sensor control device 5002 is initially installed in the sensor applicator 102, the sheath arms 5604 may be received within the first detent 5702a. As discussed below, the sheath 212 may be actuated to move the sheath arms 5604 to the second detent 5702b, which places the sensor applicator 102 in firing position.

In FIG. 26B, the applicator cap 210 is aligned with the housing 208 and advanced toward the housing 208 so that the sheath 212 is received within the applicator cap 210. Instead of rotating the applicator cap 210 relative to the housing 208, the threads of the applicator cap 210 may be snapped onto the corresponding threads of the housing 208 to couple the applicator cap 210 to the housing 208. Axial cuts or slots 5703 (one shown) defined in the applicator cap 210 may allow portions of the applicator cap 210 near its threading to flex outward to be snapped into engagement with the threading of the housing 208. As the applicator cap 210 is snapped to the housing 208, the sensor cap 5018 may correspondingly be snapped into the cap post 5314.

Similar to the embodiment of FIGS. 22A-22C, the sensor applicator 102 may include a sheath locking mechanism configured to ensure that the sheath 212 does not prematurely collapse during a shock event. In the illustrated embodiment, the sheath locking mechanism includes one or more ribs 5704 (one shown) defined near the base of the sheath 212 and configured to interact with one or more ribs 5706 (two shown) and a shoulder 5708 defined near the base of the applicator cap 210. The ribs 5704 may be configured to inter-lock between the ribs 5706 and the shoulder 5708 while attaching the applicator cap 210 to the housing 208. More specifically, once the applicator cap 210 is snapped onto the housing 208, the applicator cap 210 may be rotated (e.g., clockwise), which locates the ribs 5704 of the sheath 212 between the ribs 5706 and the shoulder 5708 of the applicator cap 210 and thereby “locks” the applicator cap 210 in place until the user reverse rotates the applicator cap 210 to remove the applicator cap 210 for use. Engagement of the ribs 5704 between the ribs 5706 and the shoulder 5708 of the applicator cap 210 may also prevent the sheath 212 from collapsing prematurely.

In FIG. 26C, the applicator cap 210 is removed from the housing 208. As with the embodiment of FIGS. 22A-22C, the applicator cap 210 can be removed by reverse rotating the applicator cap 210, which correspondingly rotates the cap post 5314 in the same direction and causes sensor cap 5018 to unthread from the mating member 5016, as generally described above. Moreover, detaching the sensor cap 5018 from the sensor control device 5002 exposes the distal portions of the sensor 5010 and the sharp 5012.

As the applicator cap 210 is unscrewed from the housing 208, the ribs 5704 defined on the sheath 212 may slidingly engage the tops of the ribs 5706 defined on the applicator cap 210. The tops of the ribs 5706 may provide corresponding ramped surfaces that result in an upward displacement of the sheath 212 as the applicator cap 210 is rotated, and moving the sheath 212 upward causes the sheath arms 5604 to flex out of engagement with the first detent 5702a to be received within the second detent 5702b. As the sheath 212 moves to the second detent 5702b, the radial shoulder 5614 moves out of radial engagement with the carrier arm(s) 5608, which allows the passive spring force of the spring 5612 to push upward on the sharp carrier 5306 and force the carrier arm(s) 5608 out of engagement with the groove(s) 5610. As the sharp carrier 5306 moves upward within the housing 208, the mating member 5016 may correspondingly retract until it becomes flush, substantially flush, or sub-flush with the bottom of the sensor control device 5002. At this point, the sensor applicator 102 in firing position. Accordingly, in this embodiment, removing the applicator cap 210 correspondingly causes the mating member 5016 to retract.

FIG. 27A is an isometric bottom view of the housing 208, according to one or more embodiments. As illustrated, one or more longitudinal ribs 5802 (four shown) may be defined within the interior of the housing 208. The ribs 5802 may be equidistantly or non-equidistantly spaced from each other and extend substantially parallel to centerline of the housing 208. The first and second detents 5702a,b may be defined on one or more of the longitudinal ribs 5802.

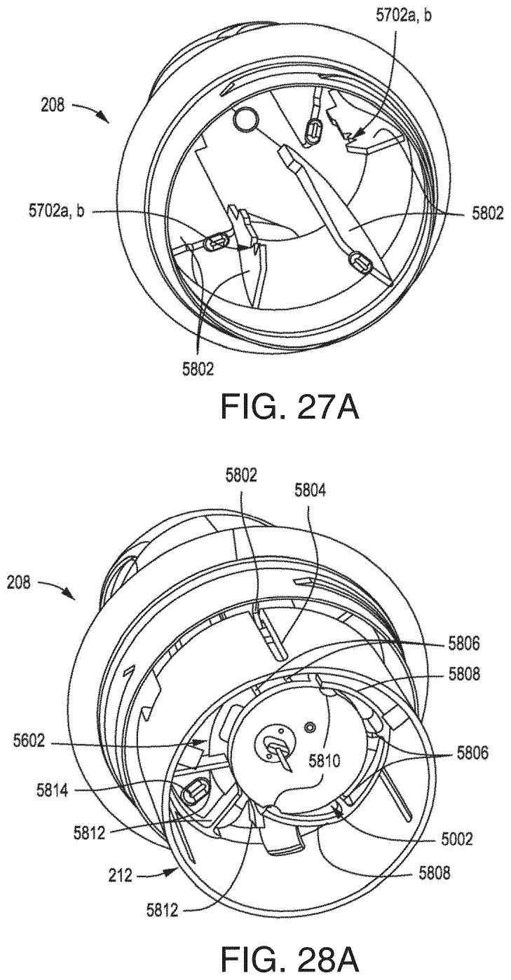

FIG. 28A is an isometric bottom view of the housing 208 with the sheath 212 and other components at least partially positioned within the housing 208. As illustrated, the sheath 212 may provide or otherwise define one or more longitudinal slots 5804 configured to mate with the longitudinal ribs 5802 of the housing 208. As the sheath 212 collapses into the housing 208, as generally described above, the ribs 5802 may be received within the slots 5804 to help maintain the sheath 212 aligned with the housing during its movement. As will be appreciated, this may result in tighter circumferential and radial alignment within the same dimensional and tolerance restrictions of the housing 208.

In the illustrated embodiment, the sensor carrier 5602 may be configured to hold the sensor control device 5002 in place both axially (e.g., once the sensor cap 5018 is removed) and circumferentially. To accomplish this, the sensor carrier 5602 may include or otherwise define one or more support ribs 5806 and one or more flexible arms 5808. The support ribs 5806 extend radially inward to provide radial support to the sensor control device 5002. The flexible arms 5808 extend partially about the circumference of the sensor control device 5002 and the ends of the flexible arms 5808 may be received within corresponding grooves 5810 defined in the side of the sensor control device 5002. Accordingly, the flexible arms 5808 may be able to provide both axial and radial support to the sensor control device 5002. In at least one embodiment, the ends of the flexible arms 5808 may be biased into the grooves 5810 of the sensor control device 5002 and otherwise locked in place with corresponding sheath locking ribs 5812 provided by the sheath 212.

In some embodiments, the sensor carrier 5602 may be ultrasonically welded to the housing 208 at one or more points 5814. In other embodiments, however, the sensor carrier 5602 may alternatively be coupled to the housing 208 via a snap-fit engagement, without departing from the scope of the disclosure. This may help hold the sensor control device 5002 in place during transport and firing.

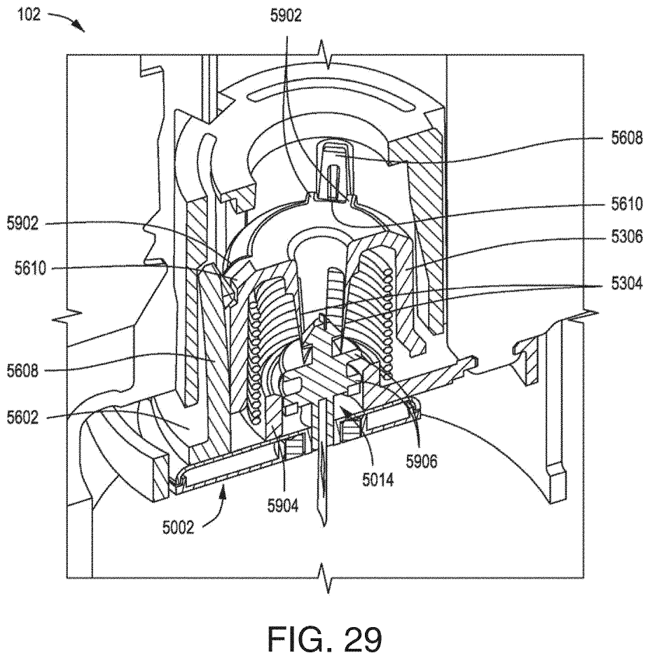

FIG. 29 is an enlarged cross-sectional side view of the sensor applicator 102 with the sensor control device 5002 installed therein, according to one or more embodiments. As discussed above, the sensor carrier 5602 may include one or more carrier arms 5608 (two shown) engageable with the sharp carrier 5306 at corresponding grooves 5610. In at least one embodiment, the grooves 5610 may be defined by pairs of protrusions 5902 defined on the sharp carrier 5306. Receiving the carrier arms 5608 within the grooves 5610 may help stabilize the sharp carrier 5306 from unwanted tilting during all stages of retraction (firing).

In the illustrated embodiment, the arms 5304 of the sharp carrier 5306 may be stiff enough to control, with greater refinement, radial and bi-axial motion of the sharp hub 5014. In some embodiments, for example, clearances between the sharp hub 5014 and the arms 5304 may be more restrictive in both axial directions as the relative control of the height of the sharp hub 5014 may be more critical to the design.

In the illustrated embodiment, the sensor carrier 5602 defines or otherwise provides a central boss 5904 sized to receive the sharp hub 5014. In some embodiments, as illustrated, the sharp hub 5014 may provide one or more radial ribs 5906 (two shown). In at least one embodiment, the inner diameter of the central boss 5904 helps provide radial and tilt support to the sharp hub 5014 during the life of sensor applicator 102 and through all phases of operation and assembly. Moreover, having multiple radial ribs 5906 increases the length-to-width ratio of the sharp hub 5014, which also improves support against tilting.

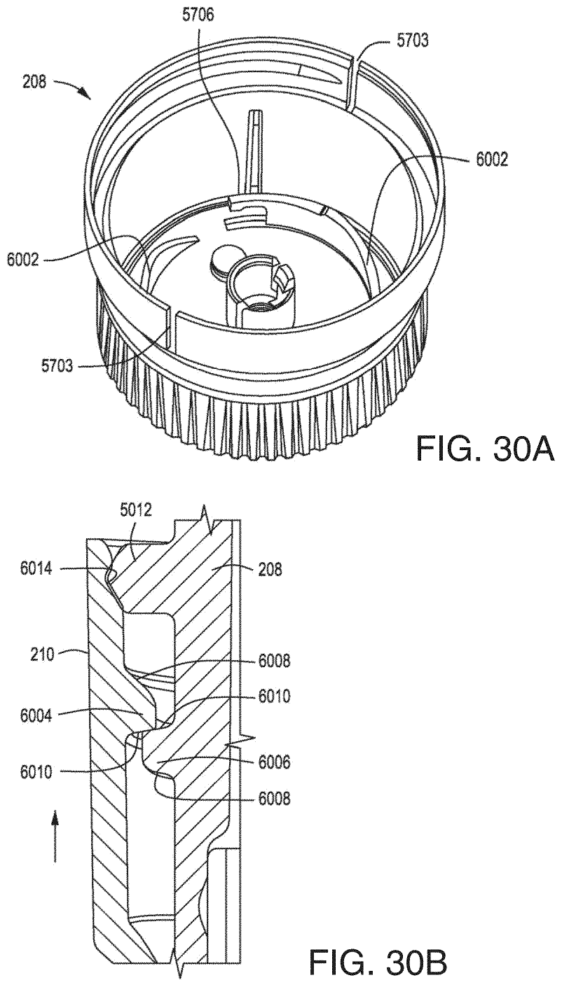

FIG. 30A is an isometric top view of the applicator cap 210, according to one or more embodiments. In the illustrated embodiment, two axial slots 5703 are depicted that separate upper portions of the applicator cap 210 near its threading. As mentioned above, the slots 5703 may help the applicator cap 210 flex outward to be snapped into engagement with the housing 208 (FIG. 26B). In contrast, the applicator cap 210 may be twisted (unthreaded) off the housing 208 by an end user.

FIG. 60 A also depicts the ribs 5706 (one visible) defined by the applicator cap 210. By interlocking with the ribs 5704 (FIG. 26C) defined on the sheath 212 (FIG. 26C), the ribs 5706 may help lock the sheath 212 in all directions to prevent premature collapse during a shock or drop event. The sheath 212 may be unlocked when the user unscrews the applicator cap 210 from the housing (FIG. 29C), as generally described above. As mentioned herein, the top of each rib 5706 may provide a corresponding ramped surface 6002, and as the applicator cap 210 is rotated to unthread from the housing 208, the ribs 5704 defined on the sheath 212 may slidingly engage the ramped surfaces 6002, which results in the upward displacement of the sheath 212 into the housing 208.

In some embodiments, additional features may be provided within the interior of the applicator cap 210 to hold a desiccant component that maintains proper moisture levels through shelf life. Such additional features may be snaps, posts for press-fitting, heat-staking, ultrasonic welding, etc.

FIG. 30B is an enlarged cross-sectional view of the engagement between the applicator cap 210 and the housing 208, according to one or more embodiments. As illustrated, the applicator cap 210 may define a set of inner threads 6004 and the housing 208 may define a set of outer threads 6006 engageable with the inner threads 6004. As mentioned herein, the applicator cap 210 may be snapped onto the housing 208, which may be accomplished by advancing the inner threads 6004 axially past the outer threads 6006 in the direction indicated by the arrow, which causes the applicator cap 210 to flex outward. To help ease this transition, as illustrated, corresponding surfaces 6008 of the inner and outer threads 6004, 6006 may be curved, angled, or chamfered. Corresponding flat surfaces 6010 may be provided on each thread 6004, 6006 and configured to matingly engage once the applicator cap 210 is properly snapped into place on the housing 208. The flat surfaces 6010 may slidingly engage one another as the user unthreads the applicator cap 210 from the housing 208.

The threaded engagement between the applicator cap 210 and the housing 208 results in a sealed engagement that protects the inner components against moisture, dust, etc. In some embodiments, the housing 208 may define or otherwise provide a stabilizing feature 6012 configured to be received within a corresponding groove 1914 defined on the applicator cap 210. The stabilizing feature 6012 may help stabilize and stiffen the applicator cap 210 once the applicator cap 210 is snapped onto the housing 208. This may prove advantageous in providing additional drop robustness to the sensor applicator 102. This may also help increase the removal torque of the applicator cap 210.

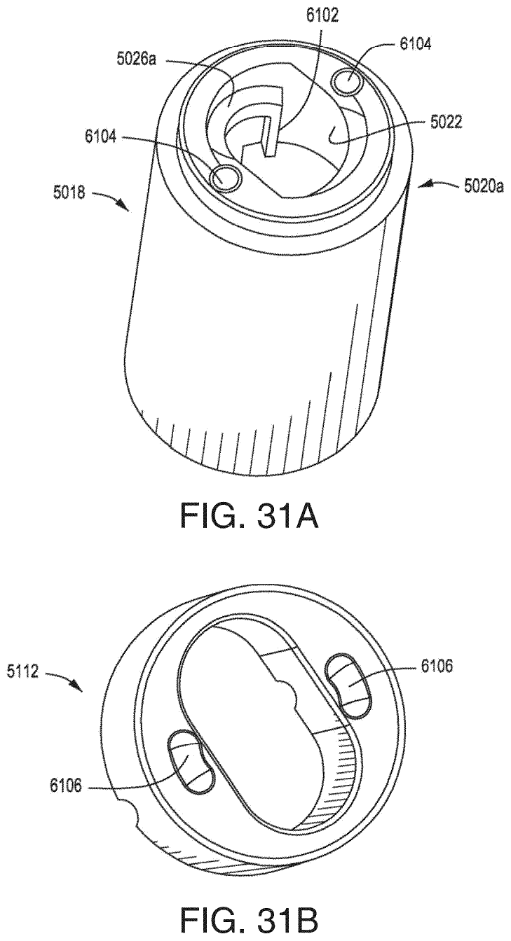

FIGS. 31A and 31B are isometric views of the sensor cap 5018 and the collar 5112, respectively, according to one or more embodiments. Referring to FIG. 31A, in some embodiments, the sensor cap 5018 may comprise an injection molded part. This may prove advantageous in molding the internal threads 5026a defined within the inner chamber 5022, as opposed to installing a threaded core or threading the inner chamber 5022. In some embodiments, one or more stop ribs 6102 (on visible) may be defined within the inner chamber 5022 to prevent over travel relative to mating member 5016 of the sharp hub 5014 (FIGS. 19A-19B).

Referring to both FIGS. 31A and 31B, in some embodiments, one or more protrusions 6104 (two shown) may be defined on the first end 5020a of the sensor cap 5018 and configured to mate with one or more corresponding indentations 6106 (two shown) defined on the collar 5112. In other embodiments, however, the protrusions 6104 may instead be defined on the collar 5112 and the indentations 6106 may be defined on the sensor cap 5018, without departing from the scope of the disclosure.

The mateable protrusions 6104 and indentations 6106 may prove advantageous in rotationally locking the sensor cap 5018 to prevent unintended unscrewing of the sensor cap 5018 from the collar 5112 (and thus the sensor control device 5002) during the life of the sensor applicator 102 and through all phases of operation/assembly. In some embodiments, as illustrated, the indentations 6106 may be formed or otherwise defined in the general shape of a kidney bean. This may prove advantageous in allowing for some over-rotation of the sensor cap 5018 relative to the collar 5112. Alternatively, the same benefit may be achieved via a flat end threaded engagement between the two parts.

### Embodiments Disclosed Herein Include:

A. A sensor control device that includes an electronics housing, a sensor arranged within the electronics housing and having a tail extending from a bottom of the electronics housing, a sharp extending through the electronics housing and having a sharp tip extending from the bottom of the electronics housing, and a sensor cap removably coupled at the bottom of the electronics housing and defining a sealed inner chamber that receives the tail and the sharp.

B. An analyte monitoring system that includes a sensor applicator, a sensor control device positioned within the sensor applicator and including an electronics housing, a sensor arranged within the electronics housing and having a tail extending from a bottom of the electronics housing, a sharp extending through the electronics housing and having a sharp tip extending from the bottom of the electronics housing, and a sensor cap removably coupled at the bottom of the electronics housing and defining an engagement feature and a sealed inner chamber that receives the tail and the sharp. The analyte monitoring system may further include a cap coupled to the sensor applicator and providing a cap post defining a receiver feature that receives the engagement feature upon coupling the cap to the sensor applicator, wherein removing the cap from the sensor applicator detaches the sensor cap from the electronics housing and thereby exposes the tail and the sharp tip.

C. A method of preparing an analyte monitoring system that includes loading a sensor control device into a sensor applicator, the sensor control device including an electronics housing, a sensor arranged within the electronics housing and having a tail extending from a bottom of the electronics housing, a sharp extending through the electronics housing and having a sharp tip extending from the bottom of the electronics housing, and a sensor cap removably coupled at the bottom of the electronics housing and defining a sealed inner chamber that receives the tail and the sharp. The method further including securing a cap to the sensor applicator, sterilizing the sensor control device with gaseous chemical sterilization while the sensor control device is positioned within the sensor applicator, and isolating the tail and the sharp tip within the inner chamber from the gaseous chemical sterilization.

Each of embodiments A, B, and C may have one or more of the following additional elements in any combination: Element 1: wherein the sensor cap comprises a cylindrical body having a first end that is open to access the inner chamber, and a second end opposite the first end and providing an engagement feature engageable with a cap of a sensor applicator, wherein removing the cap from the sensor applicator correspondingly removes the sensor cap from the electronics housing and thereby exposes the tail and the sharp tip. Element 2: wherein the electronics housing includes a shell mateable with a mount, the sensor control device further comprising a sharp and sensor locator defined on an inner surface of the shell, and a collar received about the sharp and sensor locator, wherein the sensor cap is removably coupled to the collar. Element 3: wherein the sensor cap is removably coupled to the collar by one or more of an interference fit, a threaded engagement, a frangible member, and a frangible substance. Element 4: wherein an annular ridge circumscribes the sharp and sensor locator and the collar provides a column and an annular shoulder extending radially outward from the column, and wherein a seal member interposes the annular shoulder and the annular ridge to form a sealed interface. Element 5: wherein the annular ridge defines a groove and a portion of the sensor is seated within the groove, and wherein the seal member extends into the groove to seal about the portion of the sensor. Element 6: wherein the seal member is a first seal member, the sensor control device further comprising a second seal member interposing the annular shoulder and a portion of the mount to form a sealed interface. Element 7: wherein the electronics housing includes a shell mateable with a mount, the sensor control device further comprising a sharp hub that carries the sharp and is engageable with a top surface of the shell, and a mating member defined by the sharp hub and extending from the bottom of the electronics housing, wherein the sensor cap is removably coupled to the mating member. Element 8: further comprising a collar at least partially receivable within an aperture defined in the mount and sealingly engaging the sensor cap and an inner surface of the shell. Element 9: wherein a seal member interposes the collar and the inner surface of the shell to form a sealed interface. Element 10: wherein the collar defines a groove and a portion of the sensor is seated within the groove, and wherein the seal member extends into the groove to seal about the portion of the sensor.

Element 11: wherein the receiver feature comprises one or more compliant members that flex to receive the engagement feature, and wherein the one or more compliant members prevent the engagement feature from exiting the cap post upon removing the cap from the sensor applicator. Element 12: further comprising a ramped surface defined on at least one of the one or more compliant members, and one or more camming surfaces provided by the engagement feature and engageable with the ramped surface, wherein the ramped surface and the one or more camming surfaces allow the cap and the cap post to rotate relative to the sensor cap in a first direction, but prevent the cap and the cap post from rotating relative to the sensor cap in a second direction opposite the first direction. Element 13: wherein the electronics housing includes a shell mateable with a mount, the sensor control device further comprising a sharp hub that carries the sharp and is engageable with a top surface of the shell, and a mating member defined by the sharp hub and extending from the bottom of the electronics housing, wherein the sensor cap is removably coupled to the mating member and rotating the cap in the second direction detaches the sensor cap from the mating member. Element 14: wherein the electronics housing includes a shell mateable with a mount and the sensor control device further includes a sharp and sensor locator defined on an inner surface of the shell, and a collar received about the sharp and sensor locator, wherein the sensor cap is removably coupled to the collar.

Element 15: wherein the cap provides a cap post defining a receiver feature and the sensor cap defines an engagement feature, the method further comprising receiving the engagement feature with the receiver feature as the cap is secured to the sensor applicator. Element 16: further comprising removing the cap from the sensor applicator, and engaging the engagement feature on the receiver feature as the cap is being removed and thereby detaching the sensor cap from the electronics housing and exposing the tail and the sharp tip. Element 17: wherein loading the sensor control device into a sensor applicator is preceded by sterilizing the tail and the sharp tip with radiation sterilization, and sealing the tail and the sharp tip within the inner chamber.

By way of non-limiting example, exemplary combinations applicable to A, B, and C include: Element 2 with Element 3; Element 2 with Element 4; Element 4 with Element 5; Element 4 with Element 6; Element 7 with Element 8; Element 8 with Element 9; Element 9 with Element 10; Element 11 with Element 12; and Element 15 with Element 16.

### Example Embodiments of Seal Arrangement for Analyte Monitoring Systems

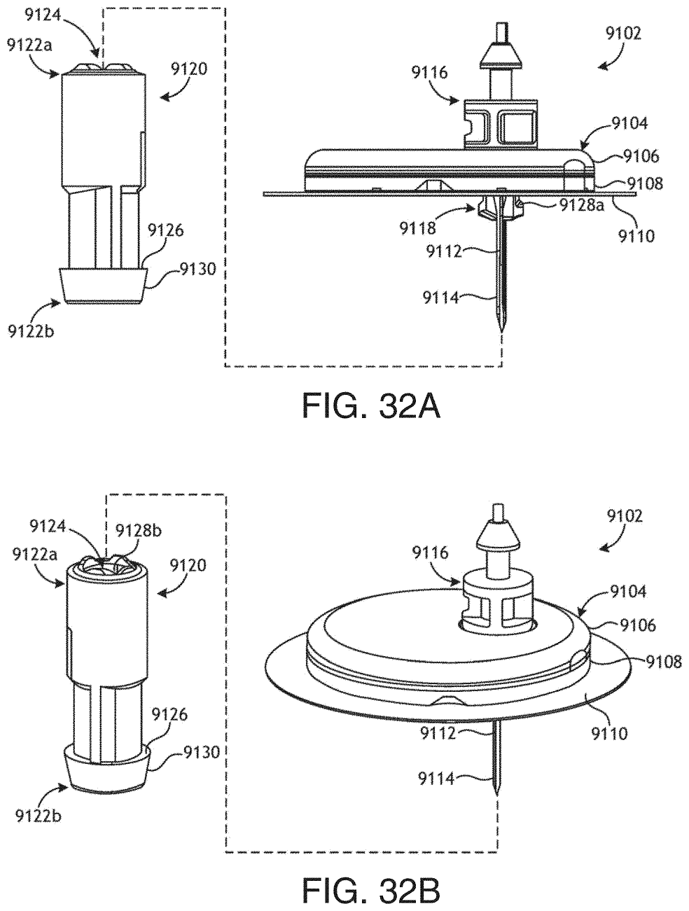

FIGS. 32A and 32B are side and isometric views, respectively, of an example sensor control device 9102, according to one or more embodiments of the present disclosure. The sensor control device 9102 may be similar in some respects to the sensor control device 102 of FIG. 1 and therefore may be best understood with reference thereto. Moreover, the sensor control device 9102 may replace the sensor control device 102 of FIG. 1 and, therefore, may be used in conjunction with the sensor applicator 102 of FIG. 1, which may deliver the sensor control device 9102 to a target monitoring location on a user's skin.

As illustrated, the sensor control device 9102 includes an electronics housing 9104, which may be generally disc-shaped and have a circular cross-section. In other embodiments, however, the electronics housing 9104 may exhibit other cross-sectional shapes, such as ovoid, oval, or polygonal, without departing from the scope of the disclosure. The electronics housing 9104 includes a shell 9106 and a mount 9108 that is mateable with the shell 9106. The shell 9106 may be secured to the mount 9108 via a variety of ways, such as a snap fit engagement, an interference fit, sonic welding, laser welding, one or more mechanical fasteners (e.g., screws), a gasket, an adhesive, or any combination thereof. In some cases, the shell 9106 may be secured to the mount 9108 such that a sealed interface is generated therebetween. An adhesive patch 9110 may be positioned on and otherwise attached to the underside of the mount 9108. Similar to the adhesive patch 108 of FIG. 1, the adhesive patch 9110 may be configured to secure and maintain the sensor control device 9102 in position on the user's skin during operation.

The sensor control device 9102 may further include a sensor 9112 and a sharp 9114 used to help deliver the sensor 9112 transcutaneously under a user's skin during application of the sensor control device 9102. Corresponding portions of the sensor 9112 and the sharp 9114 extend distally from the bottom of the electronics housing 9104 (e.g., the mount 9108). A sharp hub 9116 may be overmolded onto the sharp 9114 and configured to secure and carry the sharp 9114. As best seen in FIG. 32A, the sharp hub 9116 may include or otherwise define a mating member 9118. In assembling the sharp 9114 to the sensor control device 9102, the sharp 9114 may be advanced axially through the electronics housing 9104 until the sharp hub 9116 engages an upper surface of the electronics housing 9104 or an internal component thereof and the mating member 9118 extends distally from the bottom of the mount 9108. As described herein below, in at least one embodiment, the sharp hub 9116 may sealingly engage an upper portion of a seal overmolded onto the mount 9108. As the sharp 9114 penetrates the electronics housing 9104, the exposed portion of the sensor 9112 may be received within a hollow or recessed (arcuate) portion of the sharp 9114. The remaining portion of the sensor 9112 is arranged within the interior of the electronics housing 9104.

The sensor control device 9102 may further include a sensor cap 9120, shown detached from the electronics housing 9104 in FIGS. 32A-32B. The sensor cap 9120 may help provide a sealed barrier that surrounds and protects exposed portions of the sensor 9112 and the sharp 9114. As illustrated, the sensor cap 9120 may comprise a generally cylindrical body having a first end 9122a and a second end 9122b opposite the first end 9122a. The first end 9122a may be open to provide access into an inner chamber 9124 defined within the body. In contrast, the second end 9122b may be closed and may provide or otherwise define an engagement feature 9126. As described in more detail below, the engagement feature 9126 may help mate the sensor cap 9120 to an applicator cap of a sensor applicator (e.g., the sensor applicator 102 of FIG. 1), and may help remove the sensor cap 9120 from the sensor control device 9102 upon removing the sensor cap from the sensor applicator.

The sensor cap 9120 may be removably coupled to the electronics housing 9104 at or near the bottom of the mount 9108. More specifically, the sensor cap 9120 may be removably coupled to the mating member 9118, which extends distally from the bottom of the mount 9108. In at least one embodiment, for example, the mating member 9118 may define a set of external threads 9128a (FIG. 32A) mateable with a set of internal threads 9128b (FIG. 32B) defined within the inner chamber 9124 of the sensor cap 9120. In some embodiments, the external and internal threads 9128a,b may comprise a flat thread design (e.g., lack of helical curvature), but may alternatively comprise a helical threaded engagement. Accordingly, in at least one embodiment, the sensor cap 9120 may be threadably coupled to the sensor control device 9102 at the mating member 9118 of the sharp hub 9116. In other embodiments, the sensor cap 9120 may be removably coupled to the mating member 9118 via other types of engagements including, but not limited to, an interference or friction fit, or a frangible member or substance (e.g., wax, an adhesive, etc.) that may be broken with minimal separation force (e.g., axial or rotational force).

In some embodiments, the sensor cap 9120 may comprise a monolithic (singular) structure extending between the first and second ends 9122a,b. In other embodiments, however, the sensor cap 9120 may comprise two or more component parts. In the illustrated embodiment, for example, the body of the sensor cap 9120 may include a desiccant cap 9130 arranged at the second end 9122b. The desiccant cap 9130 may house or comprise a desiccant to help maintain preferred humidity levels within the inner chamber 9124. Moreover, the desiccant cap 9130 may also define or otherwise provide the engagement feature 9126 of the sensor cap 9120. In at least one embodiment, the desiccant cap 9130 may comprise an elastomeric plug inserted into the bottom end of the sensor cap 9120.

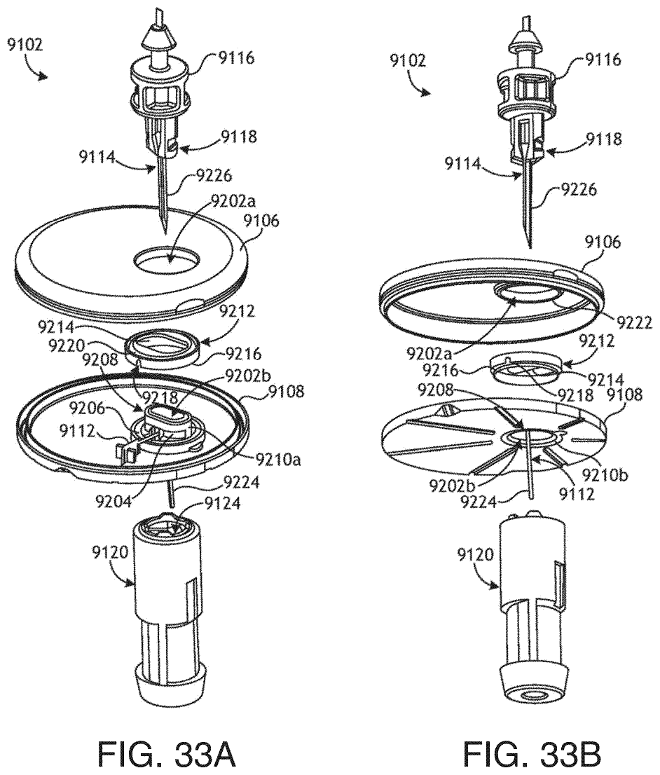

FIGS. 33A and 33B are exploded, isometric top and bottom views, respectively, of the sensor control device 9102, according to one or more embodiments. The shell 9106 and the mount 9108 operate as opposing clamshell halves that enclose or otherwise substantially encapsulate various electronic components (not shown) of the sensor control device 9102. Example electronic components that may be arranged between the shell 9106 and the mount 9108 include, but are not limited to, a battery, resistors, transistors, capacitors, inductors, diodes, and switches.

The shell 9106 may define a first aperture 9202a and the mount 9108 may define a second aperture 9202b, and the apertures 9202a,b may align when the shell 9106 is properly mounted to the mount 9108. As best seen in FIG. 33A, the mount 9108 may provide or otherwise define a pedestal 9204 that protrudes from the inner surface of the mount 9108 at the second aperture 9202b. The pedestal 9204 may define at least a portion of the second aperture 9202b. Moreover, a channel 9206 may be defined on the inner surface of the mount 9108 and may circumscribe the pedestal 9202. In the illustrated embodiment, the channel 9206 is circular in shape, but could alternatively be another shape, such as oval, ovoid, or polygonal.

The mount 9108 may comprise a molded part made of a rigid material, such as plastic or metal. In some embodiments, a seal 9208 may be overmolded onto the mount 9108 and may be made of an elastomer, rubber, a-polymer, or another pliable material suitable for facilitating a sealed interface. In embodiments where the mount 9108 is made of a plastic, the mount 9108 may be molded in a first “shot” of injection molding, and the seal 9208 may be overmolded onto the mount 9108 in a second “shot” of injection molding. Accordingly, the mount 9108 may be referred to or otherwise characterized as a “two-shot mount.”

In the illustrated embodiment, the seal 9208 may be overmolded onto the mount 9108 at the pedestal 9204 and also on the bottom of the mount 9108. More specifically, the seal 9208 may define or otherwise provide a first seal element 9210a overmolded onto the pedestal 9204, and a second seal element 9210b (FIG. 33B) interconnected to (with) the first seal element 9210a and overmolded onto the mount 9108 at the bottom of the mount 9108. In some embodiments, one or both of the seal elements 9210a,b may help form corresponding portions (sections) of the second aperture 9202b. While the seal 9208 is described herein as being overmolded onto the mount 9108, it is also contemplated herein that one or both of the seal elements 9210a,b may comprise an elastomeric component part independent of the mount 9208, such as an O-ring or a gasket.

The sensor control device 9102 may further include a collar 9212, which may be a generally annular structure that defines a central aperture 9214. The central aperture 9214 may be sized to receive the first seal element 9210a and may align with both the first and second apertures 9202a,b when the sensor control device 9102 is properly assembled. The shape of the central aperture 9214 may generally match the shape of the second aperture 9202b and the first seal element 9210a.

In some embodiments, the collar 9212 may define or otherwise provide an annular lip 9216 on its bottom surface. The annular lip 9216 may be sized and otherwise configured to mate with or be received into the channel 9206 defined on the inner surface of the mount 9108. In some embodiments, a groove 9218 may be defined on the annular lip 9216 and may be configured to accommodate or otherwise receive a portion of the sensor 9112 extending laterally within the mount 9108. In some embodiments, the collar 9212 may further define or otherwise provide a collar channel 9220 (FIG. 33A) on its upper surface sized to receive and otherwise mate with an annular ridge 9222 (FIG. 33B) defined on the inner surface of the shell 9106 when the sensor control device 9102 is properly assembled.

The sensor 9112 may include a tail 9224 that extends through the second aperture 9202b defined in the mount 9108 to be transcutaneously received beneath a user's skin. The tail 9224 may have an enzyme or other chemistry included thereon to help facilitate analyte monitoring. The sharp 9114 may include a sharp tip 9226 extendable through the first aperture 9202a defined by the shell 9106. As the sharp tip 9226 penetrates the electronics housing 9104, the tail 9224 of the sensor 9112 may be received within a hollow or recessed portion of the sharp tip 9226. The sharp tip 9226 may be configured to penetrate the skin while carrying the tail 9224 to put the active chemistry of the tail 9224 into contact with bodily fluids.

The sensor control device 9102 may provide a sealed subassembly that includes, among other component parts, portions of the shell 9106, the sensor 9112, the sharp 9114, the seal 9208, the collar 9212, and the sensor cap 9120. The sealed subassembly may help isolate the sensor 9112 and the sharp 9114 within the inner chamber 9124 (FIG. 33A) of the sensor cap 9120. In assembling the sealed subassembly, the sharp tip 9226 is advanced through the electronics housing 9104 until the sharp hub 9116 engages the seal 9208 and, more particularly, the first seal element 9210a. The mating member 9118 provided at the bottom of the sharp hub 9116 may extend out the second aperture 9202b in the bottom of the mount 9108, and the sensor cap 9120 may be coupled to the sharp hub 9116 at the mating member 9118. Coupling the sensor cap 9120 to the sharp hub 9116 at the mating member 9118 may urge the first end 9122a of the sensor cap 9120 into sealed engagement with the seal 9208 and, more particularly, into sealed engagement with the second seal element 9210b on the bottom of the mount 9108. In some embodiments, as the sensor cap 9120 is coupled to the sharp hub 9116, a portion of the first end 9122a of the sensor cap 9120 may bottom out (engage) against the bottom of the mount 9108, and the sealed engagement between the sensor hub 9116 and the first seal element 9210a may be able to assume any tolerance variation between features.

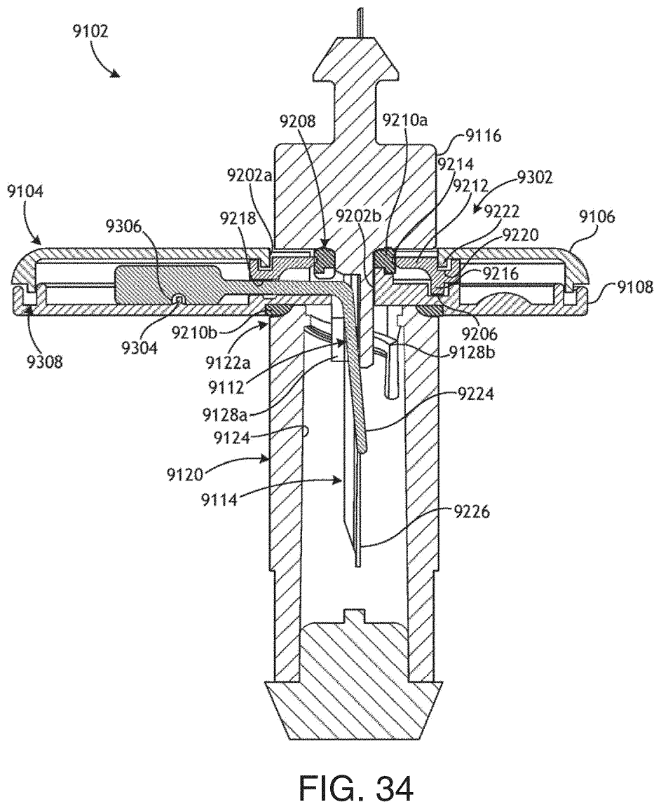

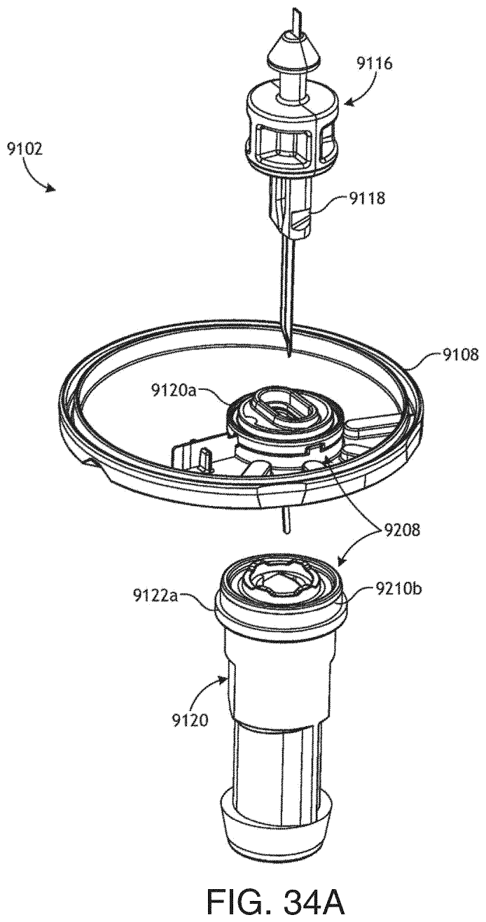

FIG. 34 is a cross-sectional side view of the sensor control device 9102, according to one or more embodiments. As indicated above, the sensor control device 9102 may include or otherwise incorporate a sealed subassembly 9302, which may be useful in isolating the sensor 9112 and the sharp 9114 within the inner chamber 9124 of the sensor cap 9120. To assemble the sealed subassembly 9302, the sensor 9112 may be located within the mount 9108 such that the tail 9224 extends through the second aperture 9202b at the bottom of the mount 9108. In at least one embodiment, a locating feature 9304 may be defined on the inner surface of the mount 9108, and the sensor 9112 may define a groove 9306 that is mateable with the locating feature 9304 to properly locate the sensor 9112 within the mount 9108.

Once the sensor 9112 is properly located, the collar 9212 may be installed on the mount 9108. More specifically, the collar 9212 may be positioned such that the first seal element 9210a of the seal 9208 is received within the central aperture 9214 defined by the collar 9212 and the first seal element 9210a generates a radial seal against the collar 9212 at the central aperture 9214. Moreover, the annular lip 9216 defined on the collar 9212 may be received within the channel 9206 defined on the mount 9108, and the groove 9218 defined through the annular lip 9216 may be aligned to receive the portion of the sensor 9112 that traverses the channel 9206 laterally within the mount 9108. In some embodiments, an adhesive may be injected into the channel 9206 to secure the collar 9212 to the mount 9108. The adhesive may also facilitate a sealed interface between the two components and generate a seal around the sensor 9112 at the groove 9218, which may isolate the tail 9224 from the interior of the electronics housing 9104.

The shell 9106 may then be mated with or otherwise coupled to the mount 9108. In some embodiments, as illustrated, the shell 9106 may mate with the mount 9108 via a tongue-and-groove engagement 9308 at the outer periphery of the electronics housing 9104. An adhesive may be injected (applied) into the groove portion of the engagement 9308 to secure the shell 9106 to the mount 9108, and also to create a sealed engagement interface. Mating the shell 9106 to the mount 9108 may also cause the annular ridge 9222 defined on the inner surface of the shell 9106 to be received within the collar channel 9220 defined on the upper surface of the collar 9212. In some embodiments, an adhesive may be injected into the collar channel 9220 to secure the shell 9106 to the collar 9212, and also to facilitate a sealed interface between the two components at that location. When the shell 9106 mates with the mount 9108, the first seal element 9210a may extend at least partially through (into) the first aperture 9202a defined in the shell 9106.

The sharp 9114 may then be coupled to the sensor control device 9102 by extending the sharp tip 9226 through the aligned first and second apertures 9202a,b defined in the shell 9106 and the mount 9108, respectively. The sharp 9114 may be advanced until the sharp hub 9116 engages the seal 9208 and, more particularly, engages the first seal element 9210a. The mating member 9118 may extend (protrude) out the second aperture 9202b at the bottom of the mount 9108 when the sharp hub 9116 engages the first seal element 9210a.

The sensor cap 9120 may then be removably coupled to the sensor control device 9102 by threadably mating the internal threads 9128b of the sensor cap 9120 with the external threads 9128a of the mating member 9118. The inner chamber 9124 may be sized and otherwise configured to receive the tail 9224 and the sharp tip 9226 extending from the bottom of the mount 9108. Moreover, the inner chamber 9124 may be sealed to isolate the tail 9224 and the sharp tip 9226 from substances that might adversely interact with the chemistry of the tail 9224. In some embodiments, a desiccant (not shown) may be present within the inner chamber 9124 to maintain proper humidity levels.

Tightening (rotating) the mated engagement between the sensor cap 9120 and the mating member 9118 may urge the first end 9122a of the sensor cap 9120 into sealed engagement with the second seal element 9210b in an axial direction (e.g., along the centerline of the apertures 9202a,b), and may further enhance the sealed interface between the sharp hub 9116 and the first seal element 9210a in the axial direction. Moreover, tightening the mated engagement between the sensor cap 9120 and the mating member 9118 may compress the first seal element 9210a, which may result in an enhanced radial sealed engagement between the first seal element 9210a and the collar 9212 at the central aperture 9214. Accordingly, in at least one embodiment, the first seal element 9210a may help facilitate axial and radial sealed engagements.

As mentioned above, the first and second seal elements 9210a,b may be overmolded onto the mount 9108 and may be physically linked or otherwise interconnected. Consequently, a single injection molding shot may flow through the second aperture 9202b of the mount 9108 to create both ends of the seal 9208. This may prove advantageous in being able to generate multiple sealed interfaces with only a single injection molded shot. An additional advantage of a two-shot molded design, as opposed to using separate elastomeric components (e.g., O-rings, gaskets, etc.), is that the interface between the first and second shots is a reliable bond rather than a mechanical seal. Hence, the effective number of mechanical sealing barriers is effectively cut in half. Moreover, a two-shot component with a single elastomeric shot also has implications to minimizing the number of two-shot components needed to achieve all the necessary sterile barriers. [0863] Once properly assembled, the sealed subassembly 9302 may be subjected to a radiation sterilization process to sterilize the sensor 9112 and the sharp 9114. The sealed subassembly 9302 may be subjected to the radiation sterilization prior to or after coupling the sensor cap 9120 to the sharp hub 9116. When sterilized after coupling the sensor cap 9120 to the sharp hub 9116, the sensor cap 9120 may be made of a material that permits the propagation of radiation therethrough. In some embodiments, the sensor cap 9120 may be transparent or translucent, but can otherwise be opaque, without departing from the scope of the disclosure.

FIG. 34A is an exploded isometric view of a portion of another embodiment of the sensor control device 9102 of FIGS. 32A-32B and 33A-33B. Embodiments included above describe the mount 9108 and the seal 9208 being manufactured via a two-shot injection molding process. In other embodiments, however, as briefly mentioned above, one or both of the seal elements 9210a,b of the seal 9208 may comprise an elastomeric component part independent of the mount 9208. In the illustrated embodiment, for example, the first seal element 9210a may be overmolded onto the collar 9212 and the second seal element 9210b may be overmolded onto the sensor cap 9120. Alternatively, the first and second seal elements 9210a,b may comprise a separate component part, such as a gasket or O-ring positioned on the collar 9212 and the sensor cap 9120, respectively. Tightening (rotating) the mated engagement between the sensor cap 9120 and the mating member 9118 may urge the second seal element 9210b into sealed engagement with the bottom of the mount 9108 in an axial direction, and may enhance a sealed interface between the sharp hub 9116 and the first seal element 9210a in the axial direction.

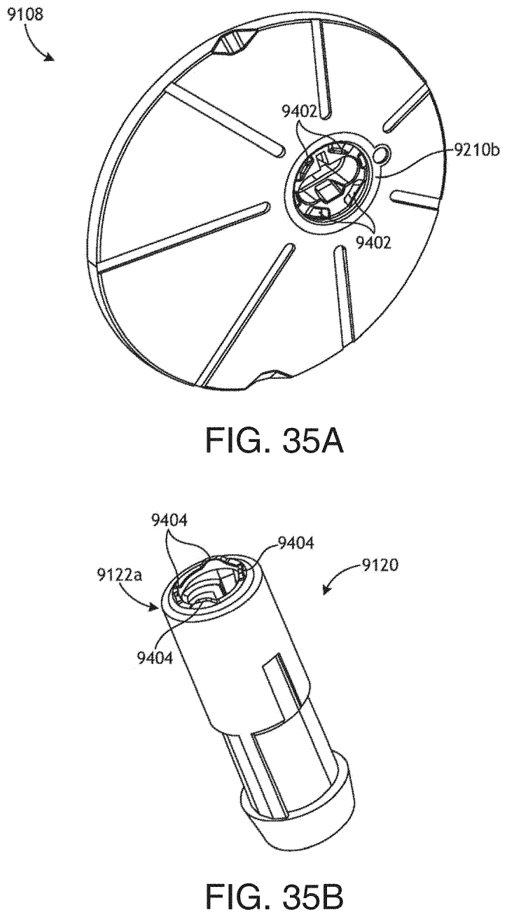

FIG. 35A is an isometric bottom view of the mount 9108, and FIG. 35B is an isometric top view of the sensor cap 9120, according to one or more embodiments. As shown in FIG. 35A, the mount 9108 may provide or otherwise define one or more indentations or pockets 9402 at or near the opening to the second aperture 9202b. As shown in FIG. 35B, the sensor cap 9120 may provide or otherwise define one or more projections 9404 at or near the first end 9122a of the sensor cap 9120. The projections 9404 may be received within the pockets 9402 when the sensor cap 9120 is coupled to the sharp hub 9116 (FIGS. 33A-33B and 93). More specifically, as described above, as the sensor cap 9120 is coupled to the mating member 9118 (FIGS. 33A-33B and 93) of the sensor hub 9116, the first end 9122a of the sensor cap 9120 is brought into sealed engagement with the second seal element 9210b. In this process, the projections 9404 may also be received within the pockets 9402, which may help prevent premature unthreading of the sensor cap 9120 from the sharp hub 9116.

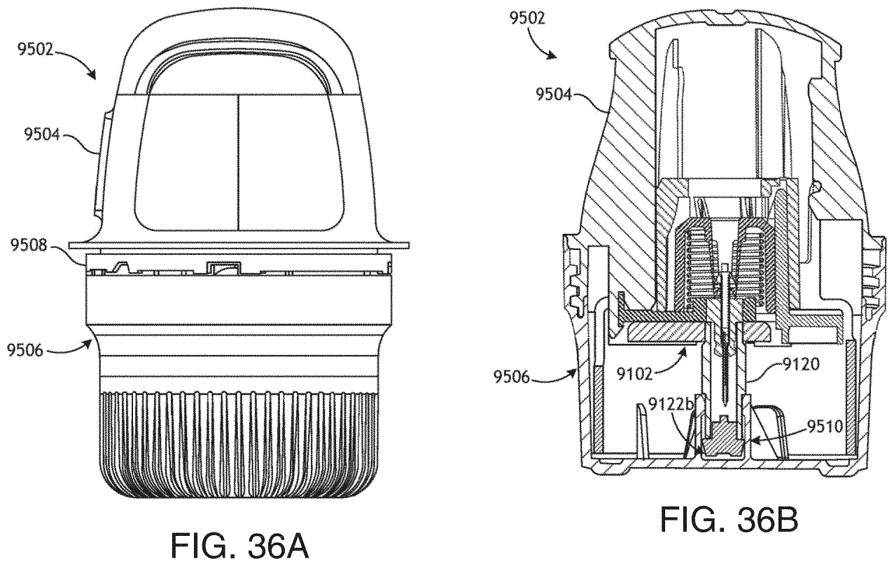

FIGS. 36A and 36B are side and cross-sectional side views, respectively, of an example sensor applicator 9502, according to one or more embodiments. The sensor applicator 9502 may be similar in some respects to the sensor applicator 102 of FIG. 1 and, therefore, may be designed to deliver (fire) a sensor control device, such as the sensor control device 9102. FIG. 36A depicts how the sensor applicator 9502 might be shipped to and received by a user, and FIG. 36B depicts the sensor control device 9102 arranged within the interior of the sensor applicator 9502.

As shown in FIG. 36A, the sensor applicator 9502 includes a housing 9504 and an applicator cap 9506 removably coupled to the housing 9504. In some embodiments, the applicator cap 9506 may be threaded to the housing 9504 and include a tamper ring 9508. Upon rotating (e.g., unscrewing) the applicator cap 9506 relative to the housing 9504, the tamper ring 9508 may shear and thereby free the applicator cap 9506 from the sensor applicator 9502.

In FIG. 36B, the sensor control device 9102 is positioned within the sensor applicator 9502. Once the sensor control device 9102 is fully assembled, it may then be loaded into the sensor applicator 9502 and the applicator cap 9506 may be coupled to the sensor applicator 9502. In some embodiments, the applicator cap 9506 and the housing 9504 may have opposing, mateable sets of threads that enable the applicator cap 9506 to be screwed onto the housing 9504 in a clockwise (or counter-clockwise) direction and thereby secure the applicator cap 9506 to the sensor applicator 9502.

Securing the applicator cap 9506 to the housing 9504 may also cause the second end 9122b of the sensor cap 9120 to be received within a cap post 9510 located within the interior of the applicator cap 9506 and extending proximally from the bottom thereof. The cap post 9510 may be configured to receive at least a portion of the sensor cap 9120 as the applicator cap 9506 is coupled to the housing 9504.

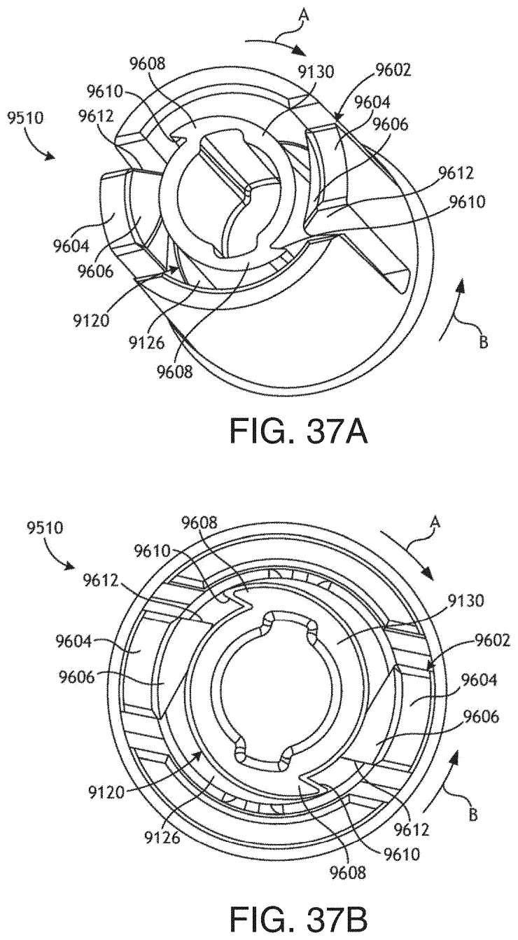

FIGS. 37A and 37B are perspective and top views, respectively, of the cap post 9510, according to one or more additional embodiments. In the illustrated depiction, a portion of the sensor cap 9120 is received within the cap post 9510 and, more specifically, the desiccant cap 9130 of the sensor cap 9120 is arranged within cap post 9510. [0871] The cap post 9510 may define a receiver feature 9602 configured to receive the engagement feature 9126 of the sensor cap 9120 upon coupling (e.g., threading) the applicator cap 9506 (FIG. 36B) to the sensor applicator 9502 (FIGS. 36A-36B). Upon removing the applicator cap 9506 from the sensor applicator 9502, however, the receiver feature 9602 may prevent the engagement feature 9126 from reversing direction and thus prevent the sensor cap 9120 from separating from the cap post 9510. Instead, removing the applicator cap 9506 from the sensor applicator 9502 will simultaneously detach the sensor cap 9120 from the sensor control device 9102 (FIGS. 32A-32B and 33A-33B), and thereby expose the distal portions of the sensor 9112 (FIGS. 33A-33B) and the sharp 9114 (FIGS. 33A-33B).

Many design variations of the receiver feature 9602 may be employed, without departing from the scope of the disclosure. In the illustrated embodiment, the receiver feature 9602 includes one or more compliant members 9604 (two shown) that are expandable or flexible to receive the engagement feature 9126. The engagement feature 9126 may comprise, for example, an enlarged head and the compliant member(s) 9604 may comprise a collet-type device that includes a plurality of compliant fingers configured to flex radially outward to receive the enlarged head.

The compliant member(s) 9604 may further provide or otherwise define corresponding ramped surfaces 9606 configured to interact with one or more opposing camming surfaces 9608 provided on the outer wall of the engagement feature 9126. The configuration and alignment of the ramped surface(s) 9606 and the opposing camming surface(s) 9608 is such that the applicator cap 9506 is able to rotate relative to the sensor cap 9120 in a first direction A (e.g., clockwise), but the cap post 9510 binds against the sensor cap 9120 when the applicator cap 9506 is rotated in a second direction B (e.g., counter clockwise). More particularly, as the applicator cap 9506 (and thus the cap post 9510) rotates in the first direction A, the camming surfaces 9608 engage the ramped surfaces 9606, which urge the compliant members 9604 to flex or otherwise deflect radially outward and results in a ratcheting effect. Rotating the applicator cap 9506 (and thus the cap post 9510) in the second direction B, however, will drive angled surfaces 9610 of the camming surfaces 9608 into opposing angled surfaces 9612 of the ramped surfaces 9606, which results in the sensor cap 9120 binding against the compliant member(s) 9604.

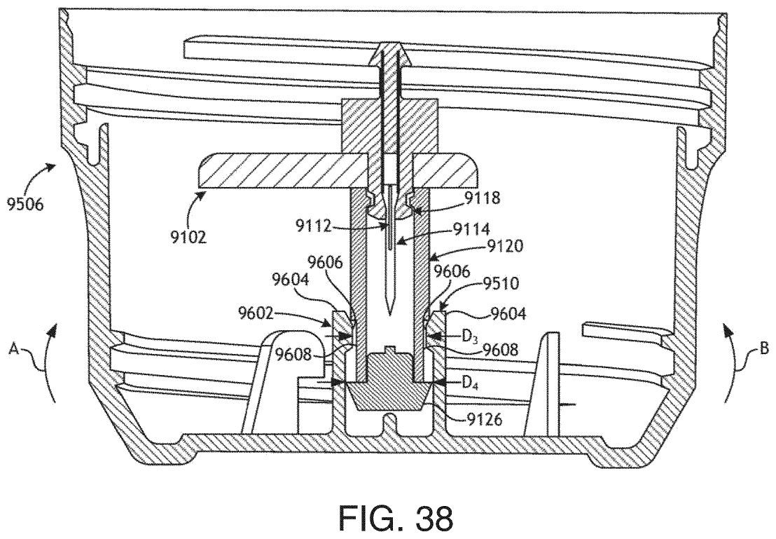

FIG. 38 is a cross-sectional side view of the sensor control device 9102 positioned within the applicator cap 9506, according to one or more embodiments. As illustrated, the opening to the receiver feature 9602 exhibits a first diameter D3, while the engagement feature 9126 of the sensor cap 9120 exhibits a second diameter D4 that is larger than the first diameter D3 and greater than the outer diameter of the remaining portions of the sensor cap 9120. As the sensor cap 9120 is extended into the cap post 9510, the compliant member(s) 9604 of the receiver feature 9602 may flex (expand) radially outward to receive the engagement feature 9126. In some embodiments, as illustrated, the engagement feature 9126 may provide or otherwise define an angled outer surface that helps bias the compliant member(s) 9604 radially outward. Once the engagement feature 9126 bypasses the receiver feature 9602, the compliant member(s) 9604 are able to flex back to (or towards) their natural state and thus lock the sensor cap 9120 within the cap post 9510.

As the applicator cap 9506 is threaded to (screwed onto) the housing 9504 (FIGS. 36A-36B) in the first direction A, the cap post 9510 correspondingly rotates in the same direction and the sensor cap 9120 is progressively introduced into the cap post 9510. As the cap post 9510 rotates, the ramped surfaces 9606 of the compliant members 9604 ratchet against the opposing camming surfaces 9608 of the sensor cap 9120. This continues until the applicator cap 9506 is fully threaded onto (screwed onto) the housing 9504. In some embodiments, the ratcheting action may occur over two full revolutions of the applicator cap 9506 before the applicator cap 9506 reaches its final position.

To remove the applicator cap 9506, the applicator cap 9506 is rotated in the second direction B, which correspondingly rotates the cap post 9510 in the same direction and causes the camming surfaces 9608 (i.e., the angled surfaces 9610 of FIGS. 37A-37B) to bind against the ramped surfaces 9606 (i.e., the angled surfaces 9612 of FIGS. 37A-37B). Consequently, continued rotation of the applicator cap 9506 in the second direction B causes the sensor cap 9120 to correspondingly rotate in the same direction and thereby unthread from the mating member 9118 to allow the sensor cap 9120 to detach from the sensor control device 9102. Detaching the sensor cap 9120 from the sensor control device 9102 exposes the distal portions of the sensor 9112 and the sharp 9114, and thus places the sensor control device 9102 in position for firing (use).

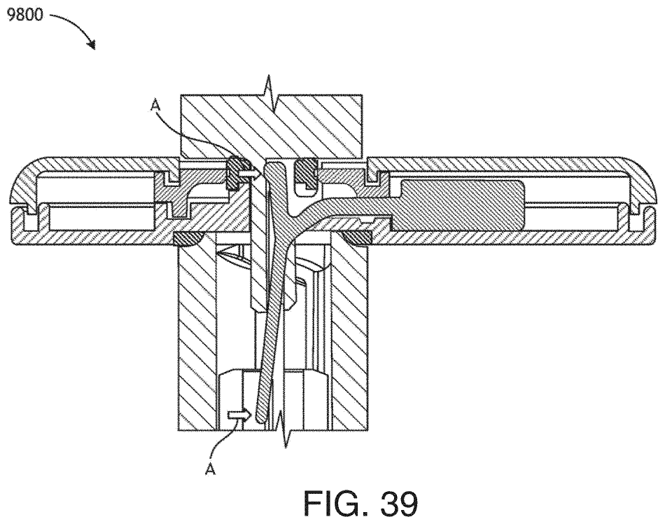

FIG. 39 is a cross-sectional view of a sensor control device 9800 showing example interaction between the sensor and the sharp. After assembly of the sharp, the sensor should sit in a channel defined by the sharp. The sensor control device in FIG. 9 does not show the sensor deflected inwards and otherwise aligned fully with the sharp, but such may be the case upon full assembly as slight bias forces may be assumed by the sensor at the locations indicated by the two arrows A. Biasing the sensor against the sharp may be advantageous so that any relative motion between the sensor and the sharp during subcutaneous insertion does not expose the sensor tip (i.e., the tail) outside the sharp channel, which could potentially cause an insertion failure.

Embodiments Disclosed Herein Include:

D. A sensor control device that includes an electronics housing including a shell that defines a first aperture and a mount that defines a second aperture alignable with the first aperture when the shell is coupled to the mount, a seal overmolded onto the mount at the second aperture and comprising a first seal element overmolded onto a pedestal protruding from an inner surface of the mount, and a second seal element interconnected with the first seal element and overmolded onto a bottom of the mount, a sensor arranged within the electronics housing and having a tail extending through the second aperture and past the bottom of the mount, and a sharp that extends through the first and second apertures and past the bottom of the electronics housing.

E. An assembly that includes a sensor applicator, a sensor control device positioned within the sensor applicator and including an electronics housing including a shell that defines a first aperture and a mount that defines a second aperture alignable with the first aperture when the shell is mated to the mount, a seal overmolded onto the mount at the second aperture and comprising a first seal element overmolded onto a pedestal protruding from an inner surface of the mount, and a second seal element interconnected with the first seal element and overmolded onto a bottom of the mount, a sensor arranged within the electronics housing and having a tail extending through the second aperture and past the bottom of the mount, and a sharp that extends through the first and second apertures and past the bottom of the electronics housing. The assembly further including a sensor cap removably coupled to the sensor control device at the bottom of the mount and defining a sealed inner chamber that receives the tail and the sharp, and an applicator cap coupled to the sensor applicator.

Each of embodiments D and E may have one or more of the following additional elements in any combination: Element 1: wherein the mount comprises a first injection molded part molded in a first shot, and the seal comprises a second injection molded part overmolded onto the first injection molded part in a second shot. Element 2: further comprising a sharp hub that carries the sharp and sealingly engages the first seal element, and a sensor cap removably coupled to the sharp hub at the bottom of the mount and sealingly engaging the second seal element, wherein the sensor cap defines an inner chamber that receives the tail and the sharp. Element 3: wherein the sharp hub provides a mating member that extends past the bottom of the mount and the sensor cap is removably coupled to the mating member. Element 4: further comprising one or more pockets defined on the bottom of the mount at the second aperture, and one or more projections defined on an end of the sensor cap and receivable within the one or more pockets when the sensor cap is coupled to the sharp hub. Element 5: further comprising a collar positioned within the electronics housing and defining a central aperture that receives and sealingly engages the first seal element in a radial direction. Element 6: further comprising a channel defined on the inner surface of the mount and circumscribing the pedestal, an annular lip defined on an underside of the collar and mateable with the channel, and an adhesive provided in the channel to secure and seal the collar to the mount at the channel. Element 7: further comprising a groove defined through the annular lip to accommodate a portion of the sensor extending laterally within the mount, wherein the adhesive seals about the sensor at the groove. Element 8: further comprising a collar channel defined on an upper surface of the collar, an annular ridge defined on an inner surface of the shell and mateable with the collar channel, and an adhesive provided in the collar channel to secure and seal the shell to the collar. Element 9: wherein one or both of the first and second seal elements define at least a portion of the second aperture. Element 10: wherein the first seal element extends at least partially through the first aperture when the shell is coupled to the mount.

Element 11: wherein the sensor control device further includes a sharp hub that carries the sharp and sealingly engages the first seal element, and wherein the sensor cap is removably coupled to the sharp hub at the bottom of the mount and sealingly engages the second seal element. Element 12: wherein the sensor control device further includes one or more pockets defined on the bottom of the mount at the second aperture, and one or more projections defined on an end of the sensor cap and receivable within the one or more pockets when the sensor cap is coupled to the sharp hub. Element 13: wherein the sensor control device further includes a collar positioned within the electronics housing and defining a central aperture that receives and sealingly engages the first seal element in a radial direction. Element 14: wherein the sensor control device further includes a channel defined on the inner surface of the mount and circumscribing the pedestal, an annular lip defined on an underside of the collar and mateable with the channel, and an adhesive provided in the channel to secure and seal the collar to the mount at the channel. Element 15: wherein the sensor control device further includes a groove defined through the annular lip to accommodate a portion of the sensor extending laterally within the mount, and wherein the adhesive seals about the sensor at the groove. Element 16: wherein the sensor control device further includes a collar channel defined on an upper surface of the collar, an annular ridge defined on an inner surface of the shell and mateable with the collar channel, and an adhesive provided in the collar channel to secure and seal the shell to the collar. Element 17: wherein one or both of the first and second seal elements define at least a portion of the second aperture. Element 18: wherein the first seal element extends at least partially through the first aperture.

By way of non-limiting example, exemplary combinations applicable to D and E include: Element 2 with Element 3; Element 2 with Element 4; Element 5 with Element 6; Element 6 with Element 7; Element 5 with Element 8; Element 11 with Element 12; Element 13 with Element 14; Element 14 with Element 15; and Element 13 with Element 16.

Additional details of suitable devices, systems, methods, components and the operation thereof along with related features are set forth in International Publication No. WO2018/136898 to Rao et. al., International Publication No. WO2019/236850 to Thomas et. al., International Publication No. W02019/236859 to Thomas et. al., International Publication No. WO2019/236876 to Thomas et. al., and U.S. patent application Ser. No. 16/433,931, filed Jun. 6, 2019, each of which is incorporated by reference in its entirety herein.

### Example Embodiments of Firing Mechanism of One Piece and Two-Piece Applicators

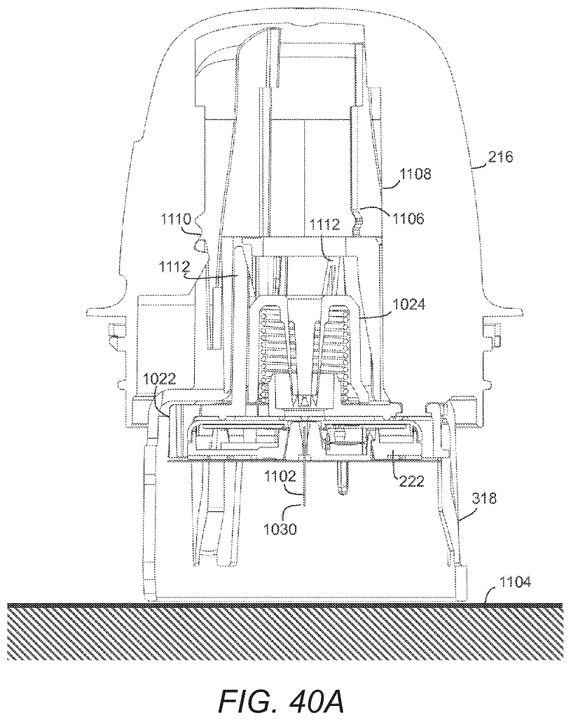

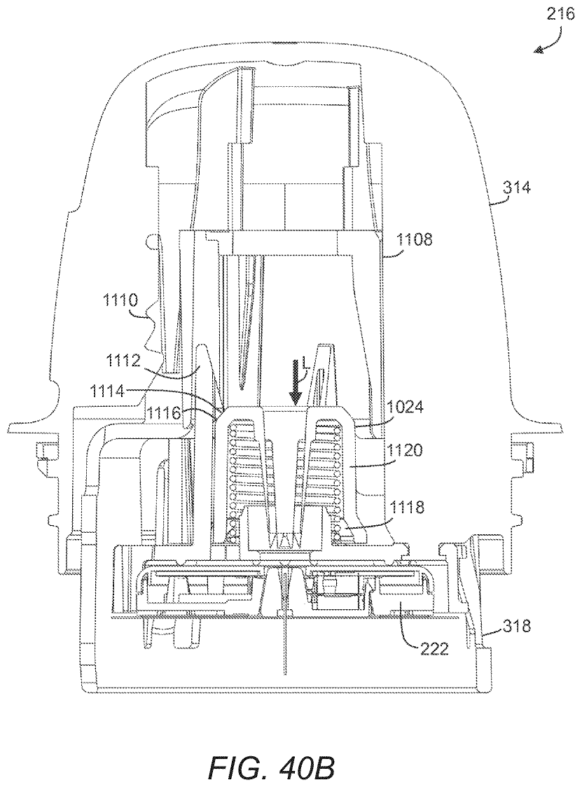

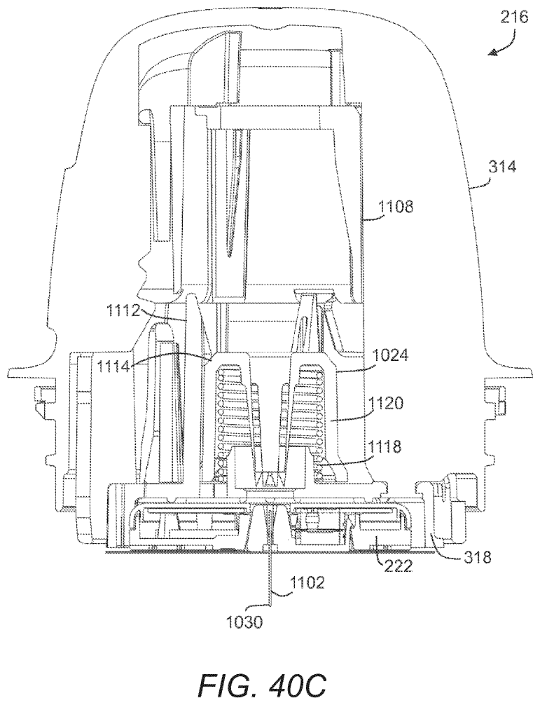

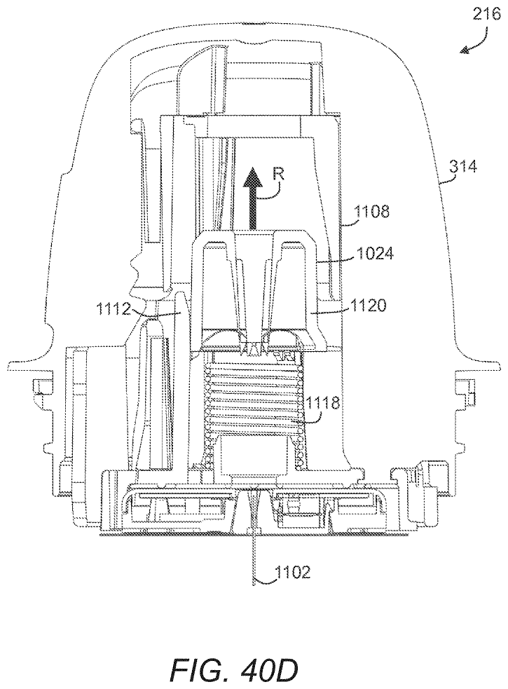

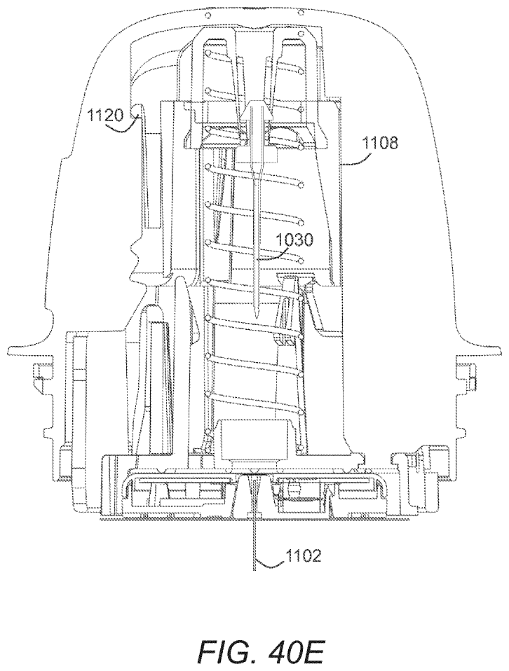

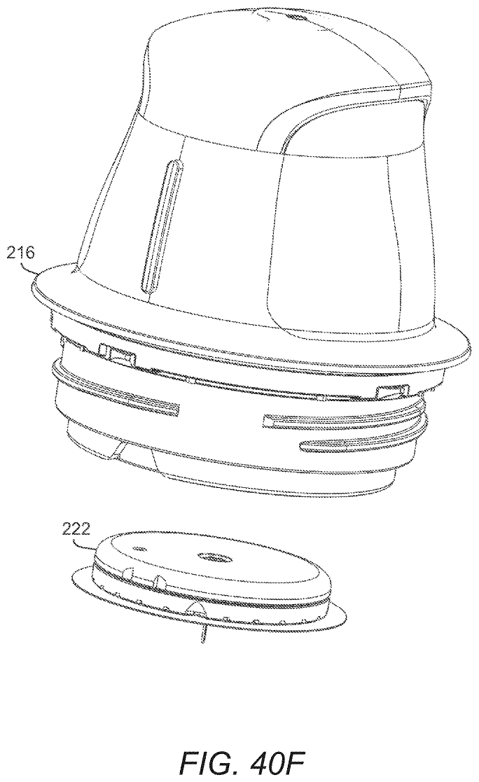

FIGS. 40A-40F illustrate example details of embodiments of the internal device mechanics of “firing” the applicator 216 to apply sensor control device 222 to a user and including retracting sharp 1030 safely back into used applicator 216. All together, these drawings represent an example sequence of driving sharp 1030 (supporting a sensor coupled to sensor control device 222) into the skin of a user, withdrawing the sharp while leaving the sensor behind in operative contact with interstitial fluid of the user, and adhering the sensor control device to the skin of the user with an adhesive. Modification of such activity for use with the alternative applicator assembly embodiments and components can be appreciated in reference to the same by those with skill in the art. Moreover, applicator 216 may be a sensor applicator having one-piece architecture or a two-piece architecture as disclosed herein.

Turning now to FIG. 40A, a sensor 1102 is supported within sharp 1030, just above the skin 1104 of the user. Rails 1106 (optionally three of them) of an upper guide section 1108 may be provided to control applicator 216 motion relative to sheath 318. The sheath 318 is held by detent features 1110 within the applicator 216 such that appropriate downward force along the longitudinal axis of the applicator 216 will cause the resistance provided by the detent features 1110 to be overcome so that sharp 1030 and sensor control device 222 can translate along the longitudinal axis into (and onto) skin 1104 of the user. In addition, catch arms 1112 of sensor carrier 1022 engage the sharp retraction assembly 1024 to maintain the sharp 1030 in a position relative to the sensor control device 222.

In FIG. 40B, user force is applied to overcome or override detent features 1110 and sheath 318 collapses into housing 314 driving the sensor control device 222 (with associated parts) to translate down as indicated by the arrow L along the longitudinal axis. An inner diameter of the upper guide section 1108 of the sheath 318 constrains the position of carrier arms 1112 through the full stroke of the sensor/sharp insertion process. The retention of the stop surfaces 1114 of carrier arms 1112 against the complimentary faces 1116 of the sharp retraction assembly 1024 maintains the position of the members with return spring 1118 fully energized.

In FIG. 40C, sensor 1102 and sharp 1030 have reached full insertion depth. In so doing, the carrier arms 1112 clear the upper guide section 1108 inner diameter. Then, the compressed force of the coil return spring 1118 drives angled stop surfaces 1114 radially outward, releasing force to drive the sharp carrier 1102 of the sharp retraction assembly 1024 to pull the (slotted or otherwise configured) sharp 1030 out of the user and off of the sensor 1102 as indicated by the arrow R in FIG. 40D.

With the sharp 1030 fully retracted as shown in FIG. 40E, the upper guide section 1108 of the sheath 318 is set with a final locking feature 1120. As shown in FIG. 40F, the spent applicator assembly 216 is removed from the insertion site, leaving behind the sensor control device 222, and with the sharp 1030 secured safely inside the applicator assembly 216. The spent applicator assembly 216 is now ready for disposal.

Operation of the applicator 216 when applying the sensor control device 222 is designed to provide the user with a sensation that both the insertion and retraction of the sharp 1030 is performed automatically by the internal mechanisms of the applicator 216. In other words, the present invention avoids the user experiencing the sensation that he is manually driving the sharp 1030 into his skin. Thus, once the user applies sufficient force to overcome the resistance from the detent features of the applicator 216, the resulting actions of the applicator 216 are perceived to be an automated response to the applicator being “triggered.” The user does not perceive that he is supplying additional force to drive the sharp 1030 to pierce his skin despite that all the driving force is provided by the user and no additional biasing/driving means are used to insert the sharp 1030. As detailed above in FIG. 40C, the retraction of the sharp 1030 is automated by the coil return spring 1118 of the applicator 216.

### Example Embodiments of Cap Seal

As seen in the figures, embodiments of one-piece applicator 150 can include housing 208 and applicator cap 210 mateable with housing 208. Applicator cap 210 provides a barrier that protects the internal contents of one-piece applicator 150. In some embodiments, applicator cap 208 may be secured to housing 208 by a threaded engagement and, upon rotating (e.g., unscrewing) applicator cap 210 relative to housing 208, applicator cap 210 can be freed from housing 208. In other embodiments, however, applicator cap 210 may be secured to housing 208 via an interference or shrink fit engagement. Consequently, to use one-piece applicator 210 for insertion of an analyte sensor, user can remove applicator cap 210 from housing 208. Furthermore, although not depicted, one-piece applicator 150 can also include any of the embodiments of applicators, sensor control units, analyte sensors, and sharps described herein, or in other publications which have been incorporated by reference.

As described herein below, the coupled engagement between housing 208 and applicator cap 210 can provide sterility to the components positioned within one-piece applicator 150 by maintaining a sterile environment as sealed with applicator cap 210. The embodiments described herein below may be applicable to analyte monitoring systems that incorporate a two-piece or a one-piece architecture. More particularly, in embodiments employing a two-piece architecture, the electronics housing (not shown) that retains the electrical components for sensor control device 102 (FIG. 1) may be positioned within housing 208 and applicator cap 210 maintains the sterile environment. In contrast, in embodiments employing a one-piece architecture, one-piece applicator 150 may contain the fully assembled sensor control device 102 (e.g., sensor control device 102 as seen in FIG. 1), and applicator cap 210 maintains the sterile environment for the fully assembled sensor control device.

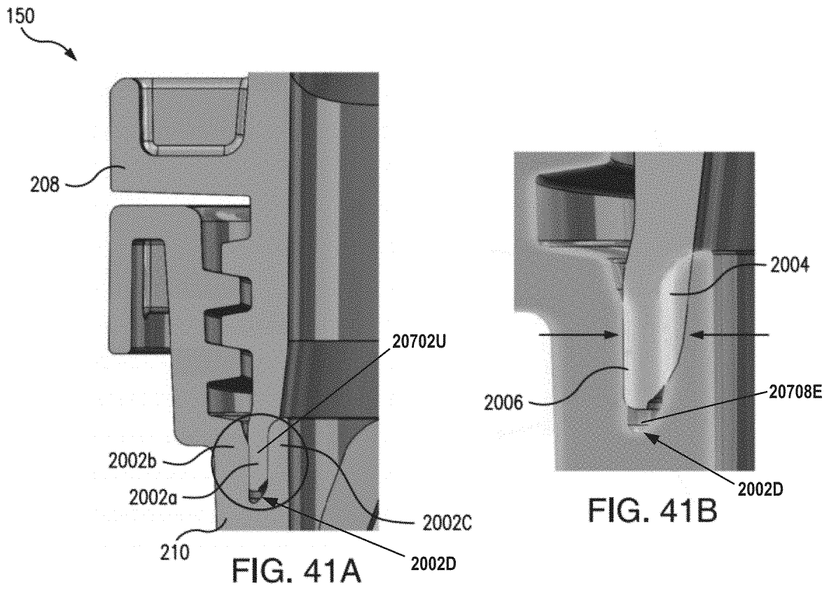

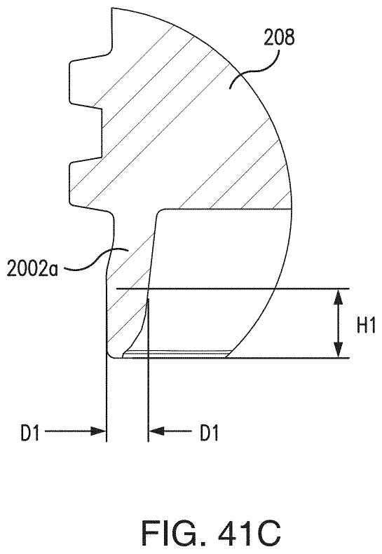

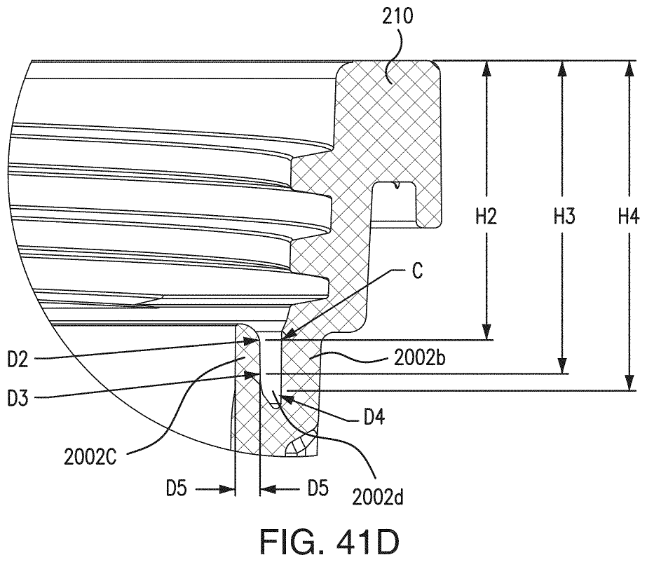

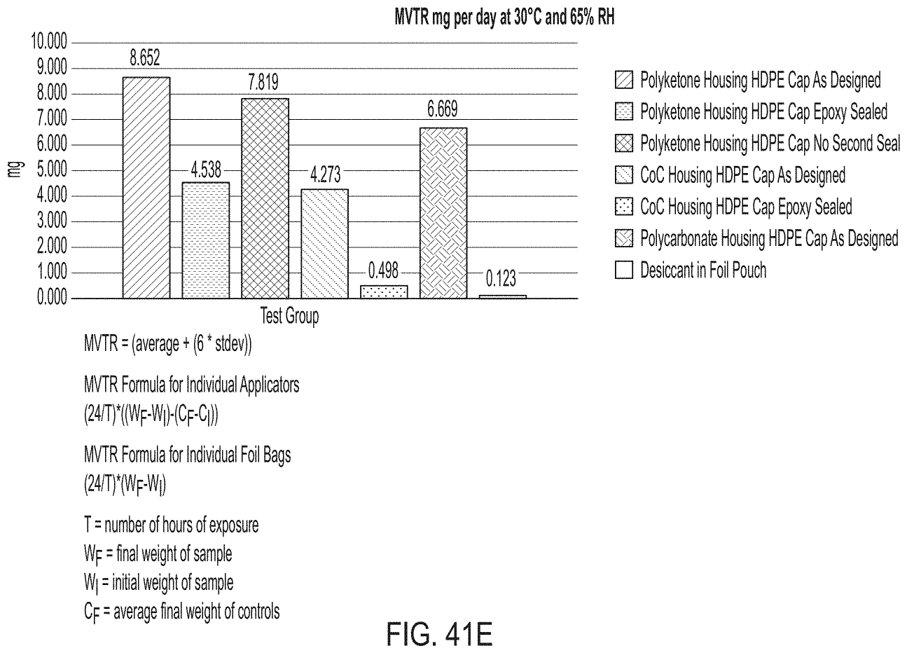

FIGS. 41A-D show an enlarged cross-sectional side view of the interface between housing 208 and applicator cap 210. As illustrated, applicator cap sealing lip 20702U of housing 208 includes a first axial extension 2002a and seal interface 20708E of cap 210 provides a cavity 2002d mateable with the first axial extension 2002a. In the illustrated embodiment, the diameter of cavity 2002d formed from second axial extension 2002b and third axial extension 2002c of cap 210 is sized to receive the diameter of first axial extension 2002a of housing 208 within cavity 2002d. For example, as shown in FIG. 41C, axial extension 2002a can have thickness D1 at height H1, as measured from distal edge of axial extension 2002a. Similarly, second axial extension 2002c can have a thickness D5 at height H3, as measured from proximal edge of cap 210; cavity 2002d can have a thickness D2, D3, and D4 at heights H2, H3, and H4, respectively, as measured from proximal edge of cap 210. In certain embodiments, D1 can measure 1 mm with a tolerance of +/−0.03 mm, D2, D3, D4 can have any suitable dimensions, D5 can measure 0.74 mm with a tolerance of +/−0.5 mm, H1 can measure 1.66 mm with a tolerance of +/−0.1 mm, H2 can measure 8.25 mm with a tolerance of +/−0.1 mm, H3 can measure 9.25 mm with a tolerance of +/−0.1 mm, H4 can measure 9.75 mm with a tolerance of +/−0.1 mm. In other embodiments, however, the reverse can be employed, where the diameter of first axial extension 2002a may be sized to receive the diameter of the second axial extension 2002b, without departing from the scope of the disclosure.

In each embodiment, two radial seals 2004, 2006 can be defined or otherwise provided at the interface between first and second axial extensions 2002a,b and radial seals 2004 and 2006 may help prevent migration of fluids or contaminants across the interface in either axial direction. Moreover, the dual radial seals described herein can accommodate tolerance and thermal variations combined with stress relaxation via a redundant sealing strategy. In the illustrated embodiment, dual radial seals 2004, 2006 utilize a “wedge” effect for effective sealing between first axial extension 2002a and second axial extension 2002b.

### Example Embodiments of Environmentally Conscious Packaging and Components

According to embodiments of the present disclosure, analyte monitoring systems that incorporate a two-piece or a one-piece architecture may be shipped to a user in a sealed package. More particularly, in embodiments employing a two-piece architecture, applicator 150 and sensor container or tray 810 can be shipped in a single sealed package. Alternatively, applicator 150 can and sensor container or tray 810 can be shipped in separate sealed packages. In contrast, in embodiments employing a one-piece architecture, one-piece applicator 150 can be shipped in a single sealed package. According to embodiments of the present disclosure, sealed package can include sealed foil bags or any other sealed package known to a person of ordinary skill in the art. The sealed package described herein can be designed to maintain a low moisture vapor transition rate (MVTR), thereby enabling stable shelf life for one-piece and two-piece analyte monitoring systems. For example, as shown in the chart depicted in FIG. 41E, the MVTR was tested at 30C and 65% relative humidity for a number of different materials and seals.

According to embodiments of the present disclosure, sealed package may be resealable. For example, sealed packaging can include resealing mechanism such as zip-type interlocking closure, or any other method or system known to a person of ordinary skill in the art.

Additionally, sealed package may include a pre-paid, pre-printed return shipping label allowing users to return used applicators, containers, and/or sensor control devices for recycling or sharps for disposal. Moreover, sealed package described herein may prove advantageous in eliminating component parts and various fabrication process steps. For example, by carefully planning humidity control during manufacturing, sealed package described herein may either eliminate the need for a desiccant or allow use of a smaller off-the-shelf desiccant within the sealed package. Furthermore, pressure decay leak testing may no longer be required during the manufacturing processes. For example, pressure decay testing is conducted during manufacturing once applicator has been assembled and packaged, as well as when sensor control device 9102 has been assembled, in case of one-piece architecture systems. As such, housing and cap are designed using material that can achieve a proper seal between components to ensure the product meets its intended shelf life. However, if a foil sealed bag is utilized, stringent pressure decay test of different components may no longer be required.

According to embodiments of the present disclosure, any of the applicator embodiments described herein, as well as any of the components thereof, including but not limited to the housing, sheath, sharp carrier, electronics carrier, firing pin, sharp hub, sensor module embodiments, actuator, and sensor container or tray may be made of a variety of rigid materials. In some embodiments, for example, the components may be made of an engineered thermoplastic, such as acetal or polyoxymethylene. Use of a single material for the construction of the various components of the applicator embodiments described herein may be advantageous in improving recyclability, lubricity, and tight tolerance control. Specifically, acetal can be used to provide lubricity (i.e., low friction) between parts which move relative to each other, for example, sheath and housing, sharp carrier and housing. As such, reducing friction can help provide sufficient force to achieve successful sensor insertion. Use of acetal can additionally reduce the need for pressure decay testing during manufacturing. In other embodiments, for example, other materials having the same or similar properties to acetal, such as polybutylene terephthalate (PBT), can be used for any or all of the aforementioned components. Additionally, use of a sealable package reduces the need for tight component tolerance control generally required to achieve a proper seal between applicator housing to cap, therefore allowing a single material to be used for manufacture. Tighter tolerance parts generally require tightly controlled tooling and processes, thereby increasing manufacturing costs for parts. Use of a single material can therefore reduce manufacturing costs. For example, after separation of any metallic components such as, drive spring, battery, and retraction spring, using a magnet, all remaining components made form the same material may be easily recycled.

### Example Embodiments of Reset Tool, Docking Station, and Reusable Applicator

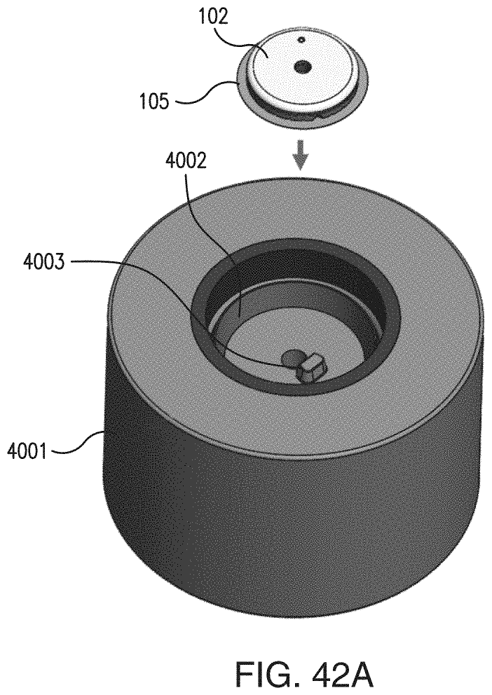

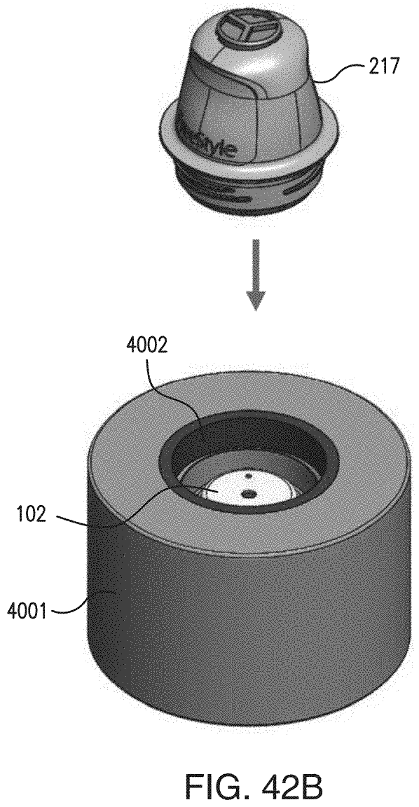

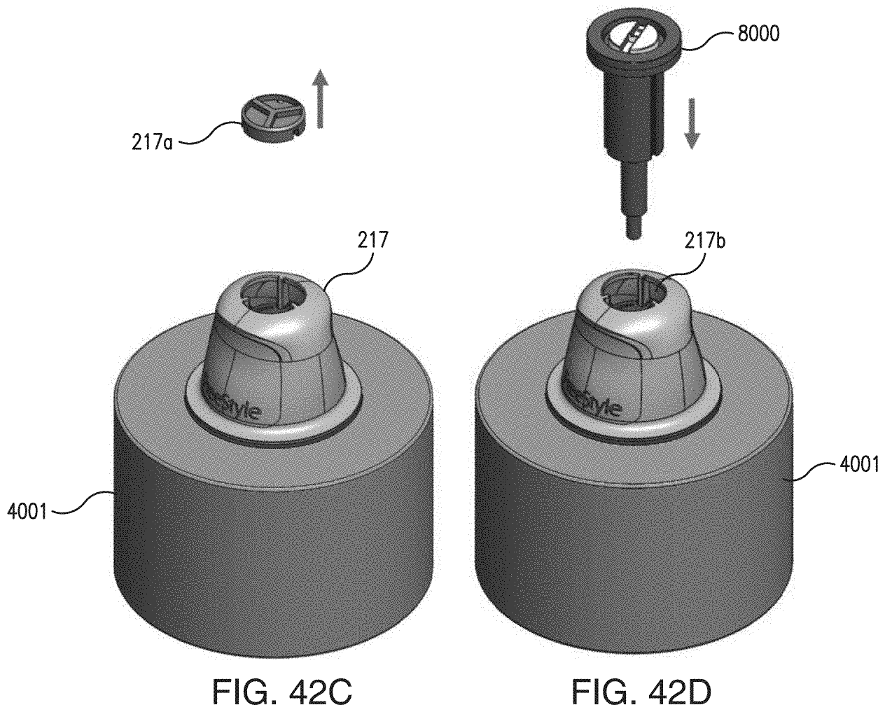

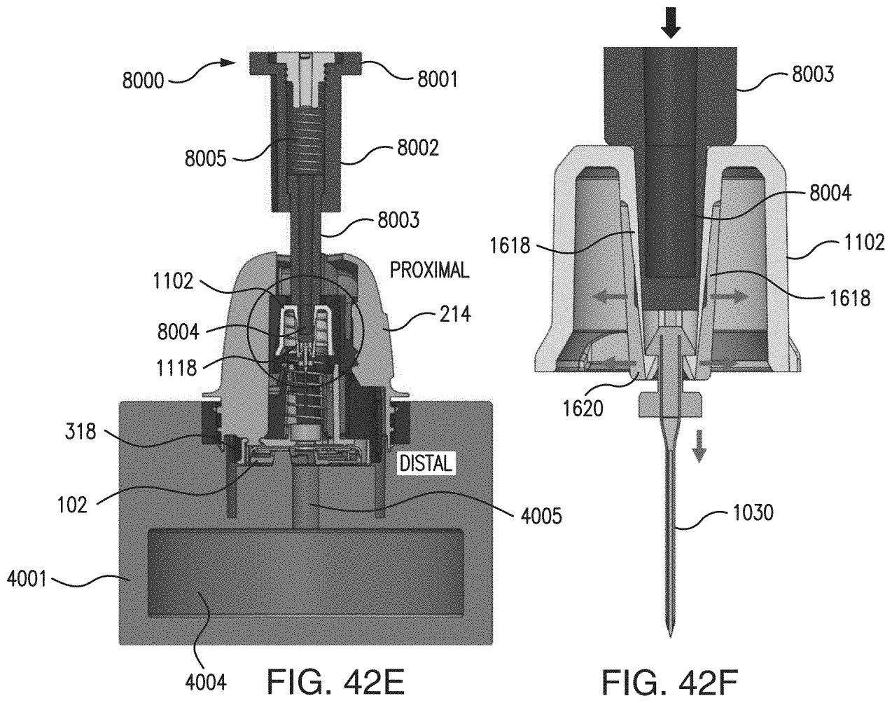

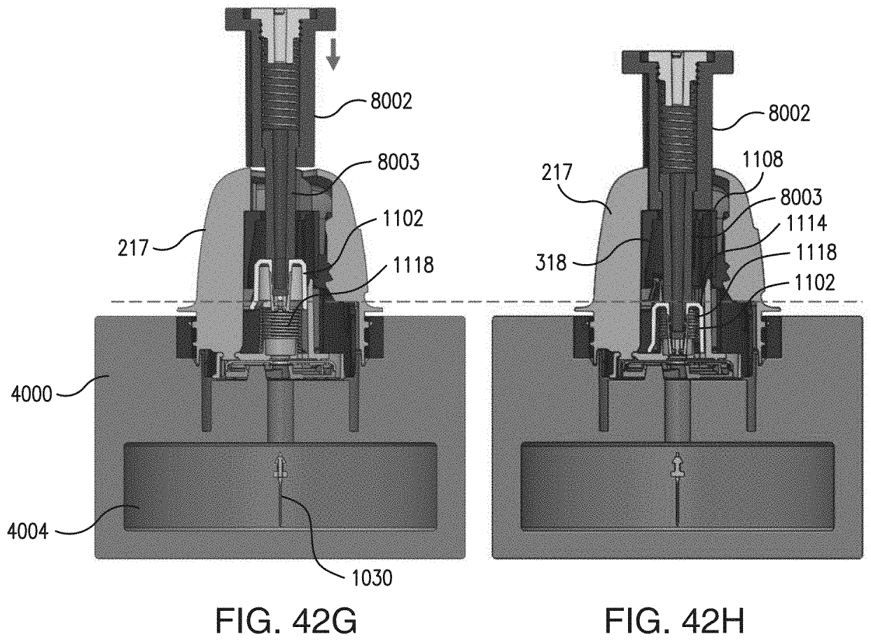

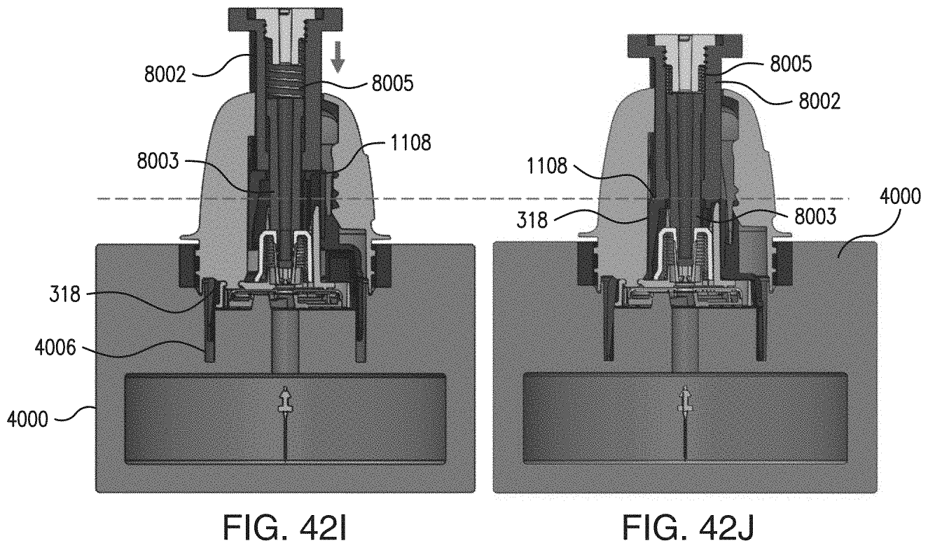

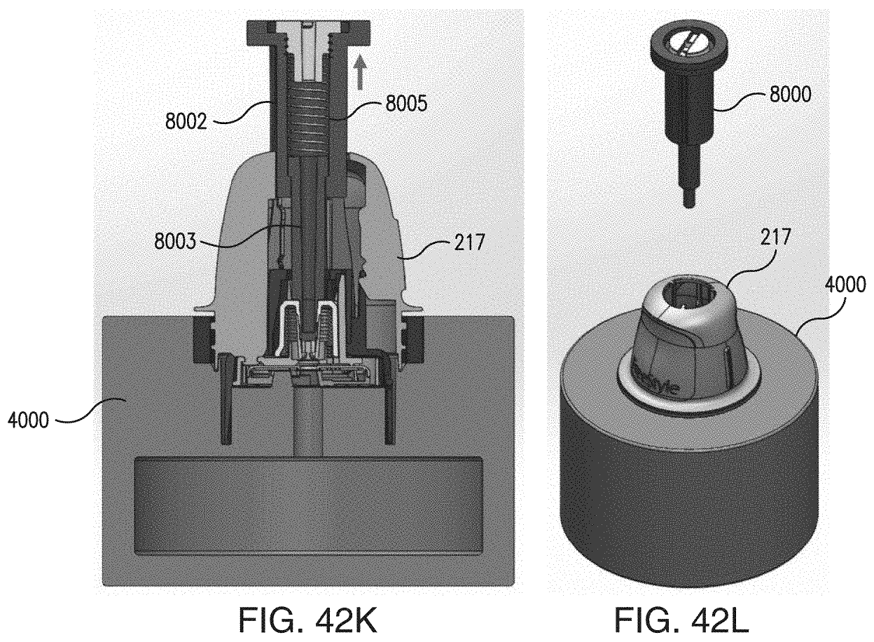

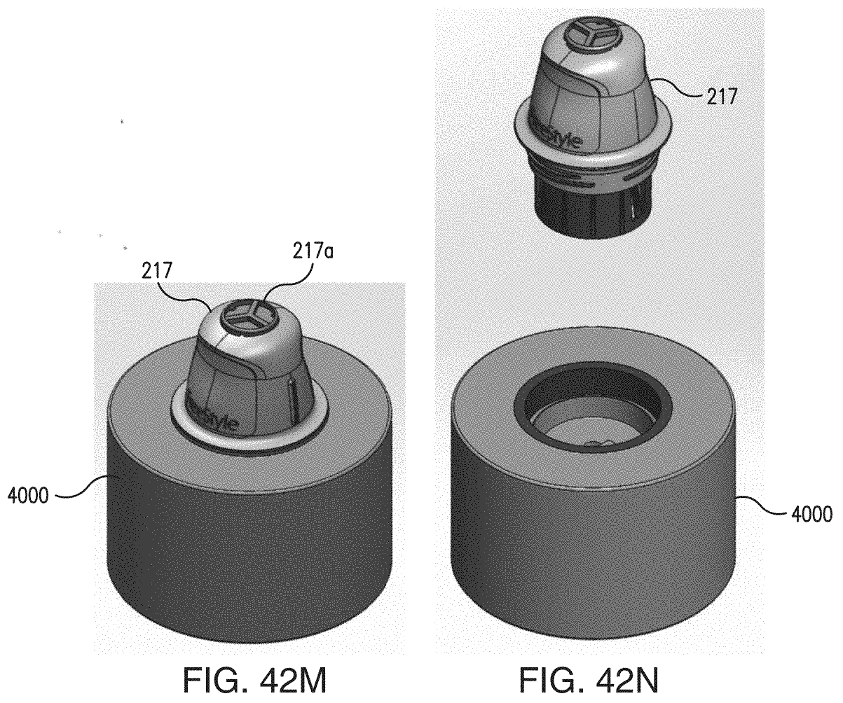

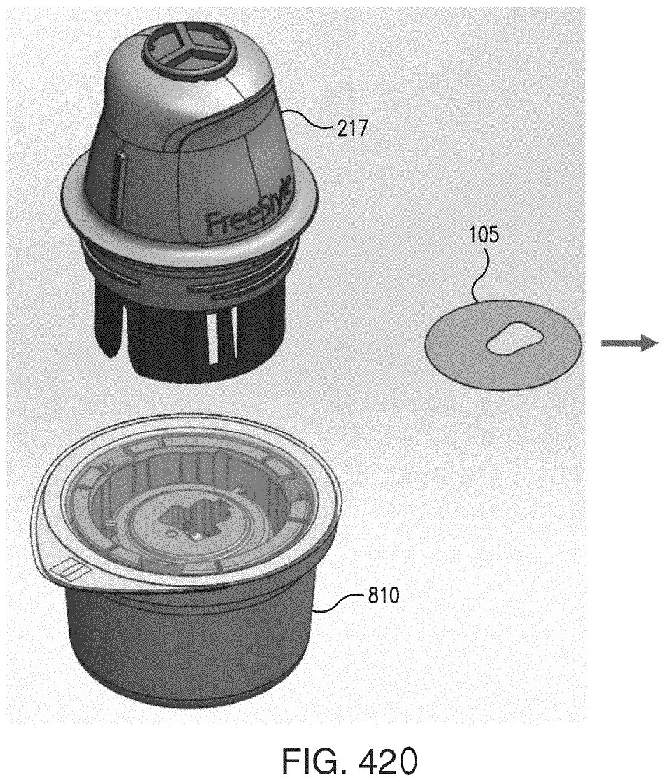

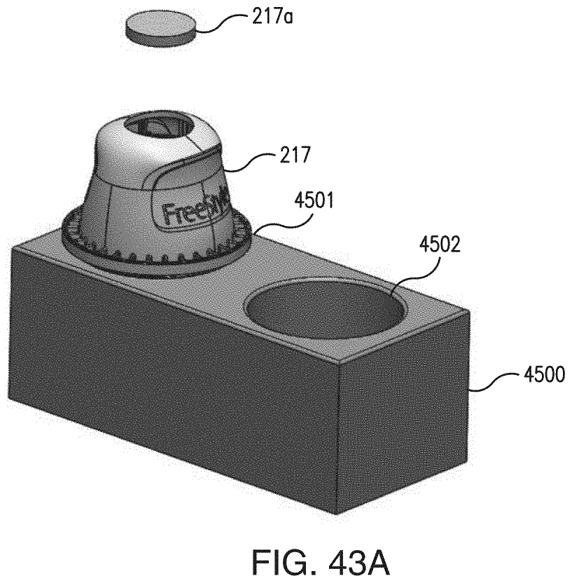

According to embodiments of the present disclosure, one-piece or two-piece architecture sensor applicators can be a reusable type. For example, as best shown in FIGS. 42A-42O, spent sensor applicator 217 (e.g., similar to spent applicator 216 shown in FIG. 40F) can be reset and reused for subsequent insertion of another analyte sensor by a user. Specifically, used sharp 1030 (e.g., as shown in FIG. 40E) can be removed from sensor applicator 217 and discarded, sharp retraction assembly 1024 can be reset and return spring 1118 reloaded, and sheath 318 can be reset so that reusable applicator 217 can be reused for insertion of a subsequent sensor 1102. Moreover, reusable applicator 217 can be any one-piece or two-piece architecture embodiments disclosed above. Furthermore, although not depicted, applicator 217 can also include any of the embodiments of sensor control units, analyte sensors, and sharps described herein, or in other publications which have been incorporated by reference. Reusable applicator 217 can be advantageous in that it can be reused, thereby reducing overall cost to consumers and environmental impact.

FIGS. 42A-42O depict an example embodiment of a reusable applicator 217 being “reset” using reset tool 8000 and docking station 4000. All together, these drawings represent an example sequence of coupling a new sensor carrier 222a to reusable applicator 217, releasing used sharp 1030 from reusable applicator 217, resetting sharp retraction assembly 1024, and resetting sheath 318. Modification of such activity for use with the alternative applicator assembly embodiments and components can be appreciated in reference to the same by those with skill in the art.

As illustrated in FIGS. 42D-421, reset tool 8000 can include a first longitudinal length, i.e., cylindrical section 8002, telescopically coupled to a second longitudinal length, i.e., cylindrical section 8003. More specifically, cylindrical section 8002 can include a traverse dimension sized and dimensioned for insertion into reusable applicator 217 and a hollow interior 8002a. As best show in FIG. 42J, cylindrical section 8003 can be sized and dimensioned to telescopically couple with cylindrical section 8002. Hollow interior 8002a can house spring 8005 configured to bias cylindrical section 8003 towards a distal end of cylindrical section 8002, as best shown in FIG. 42H. Furthermore, cylindrical section 8002 can include handle 8001 for ergonomic use of reset tool 8000. Additionally, distal end of cylindrical section 8003 can include stepped cylindrical section 8004 in axial alignment with cylindrical section 8003. Traverse dimension of cylindrical section 8003 can be sized and dimension for insertion into sheath 318, while traverse dimension of cylindrical section 8004 can be sized and dimensioned for insertion into sharp carrier 1102. Cylindrical section 8004 has a larger traverse dimension (e.g., diameter) than cylindrical section 8003, and cylindrical section 8003 has a larger traverse dimension (e.g., diameter) than cylindrical section 8004. Moreover, cylindrical sections 8003 and 8004 can be hollow, thereby reducing overall weight of reset tool 8000. While sections 8002,8003, and 8004 are shown as cylindrical, any other suitable shape could be used.

FIGS. 42D-42I illustrate example details of embodiments of mechanics of “resetting” reusable applicator 217 using reset tool 8000. In an initial step, referring to FIG. 42A and 42B, a new, unused sensor control device 102 (i.e., including adhesive patch 105) is releasably positioned in recess 4002a of channel 4002 of docking station 4000 as indicated by the arrow. Recess 4002a can include alignment feature 4003 configured to rotationally align sensor control device 102. Specifically, sensor control device 102 can include a notch corresponding to alignment feature 4003, which, when engaged with alignment feature 4003, prevents rotational movement of sensor control device 102 within channel 4002. Subsequently, spent reusable applicator 217 (e.g., applicator 216 as shown in FIG. 40F) is placed within and advanced into channel 4002, as indicated by the arrow, until sensor control device 102 couples to sensor carrier 1022. According to embodiments of the present disclosure, reusable applicator 217 can be designed to provide the user with an audible or sensory cue when control device 102 successfully couples to sensor carrier 1022.

As illustrated in FIG. 42C-D, after sensor control device 102 has been coupled to reusable applicator 217, removable plug 217a can be removed, as indicated by the arrow, to access reset channel 217b within applicator 217 and reset tool 8000 can be inserted into reset channel 217b, as indicated by the arrow, to reset applicator 217. In FIG. 42E, reset tool 8000 is inserted into reset channel 217b along longitudinal axis of applicator 217 until cylindrical section 8004 engages sharp retention arms 1618 of sharp carrier 1102. FIG. 42F illustrates an enlarged cross-sectional side view of the engagement of cylindrical section 8004 and sharp retention arms 1618. As user force is applied to advance reset tool 8000 in a distal direction into applicator 217, as indicated by the arrow along the longitudinal axis, cylindrical section 8004 causes sharp retention arms 1618 to displace radially outwards, as indicated by the arrows pointing radially outwards. Consequently, sharp retention clip 1620 releases sharp 1030 and released sharp 1030 advances through axially aligned sharp channel 4005 of docking station 4000 into collection chamber 4004, where used sharp 1030 can safely be collected and stored for subsequent disposal (as shown in FIG. 42G). Cylindrical section 8002 is advanced into sharp carrier 1102 until cylindrical section 8003 engages sharp carrier 1102.

With further reference to FIG. 42G, as user force is further applied to advance reset tool 8000 in a distal direction into applicator 217, cylindrical section 8003 drives sharp carrier 1102 towards sensor carrier 1022 until faces 1116 of sharp retraction assembly 1024 reengage stop surfaces 1114 of carrier arms 1112. As a result, as best seen in FIG. 42H, return spring 1118 is recompressed. Furthermore, retention of stop surfaces 1114 of carrier arms 1112 against complimentary faces 1116 of sharp retraction assembly 1024 maintain the position of the members with return spring 1118 fully energized. Once sharp retraction assembly 1024 is repositioned within carrier arms 1112, cylindrical section 8002 engages upper guide section 1108 of the sheath 318.

In FIG. 421-J, as user force is continued to be applied to advance reset tool 8000 in a distal direction into applicator 217, cylindrical section 8002 drives sheath 318 in a distal direction into sheath channels 4006 of docking station 4000. Additionally, as seen in FIG. 421-J, cylindrical section 8003 collapses within cylindrical section 8002 and compresses spring 8005.

As seen in FIGS. 42K-L, after sheath 318 has been fully extended out of applicator 217 in the distal direction, user force can be removed. As a result, compressed force of spring 8005 drives cylindrical section 8002 in a proximal direction, as seen in FIG. 42K. After cylindrical section 8002 has fully retracted in the proximal direction, reset tool 8000 can be removed from applicator 217, as seen in FIG. 42L. Thereafter, as seen in FIG. 42M, applicator plug 217a can be reapplied to seal reset channel 217b. At this stage, as seen in FIG. 42N-O, applicator 217 has been reset (i.e., it includes a new sensor carrier with adhesive patch) and can be removed from docking station 4000 for insertion of another analyte sensor. As best seen in FIG. 42O, to reuse applicator 217 to insert another analyte sensor, user may remove adhesive patch 105 from new sensor carrier (not shown) and engage applicator 217 with container or tray 810.

Referring again to FIGS. 42D-L, although reset tool 8000 is depicted as a separate structure, in some embodiments, reset tool can be fully or partially integrated with applicator 217. For example, according to some embodiments, reset tool 8000 can be integrated with a re-usable applicator, and further comprise an external button configured to be actuated by the user to perform a reset after a sensor insertion (e.g., when the sharp is ready to be disposed of and the sharp carrier is ready to be reset). Further details regarding embodiments of applicators, their components, and variants thereof, are described in U.S. Patent Publication No. 2013/0150691.

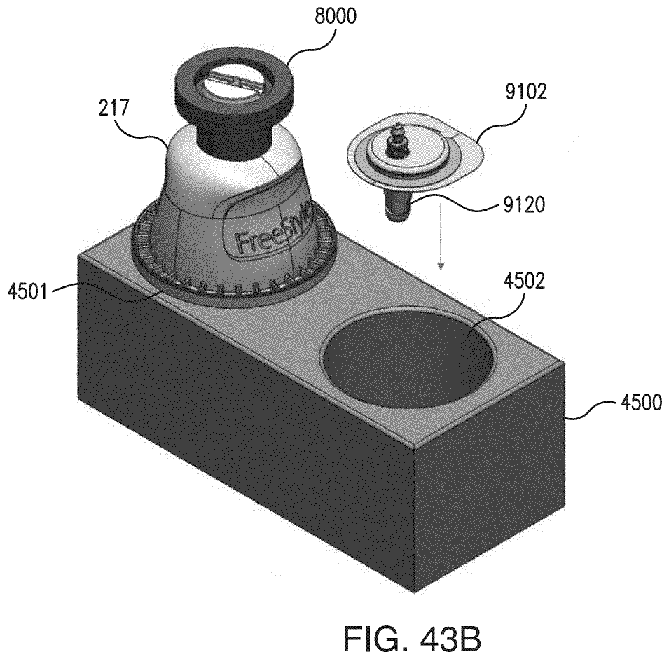

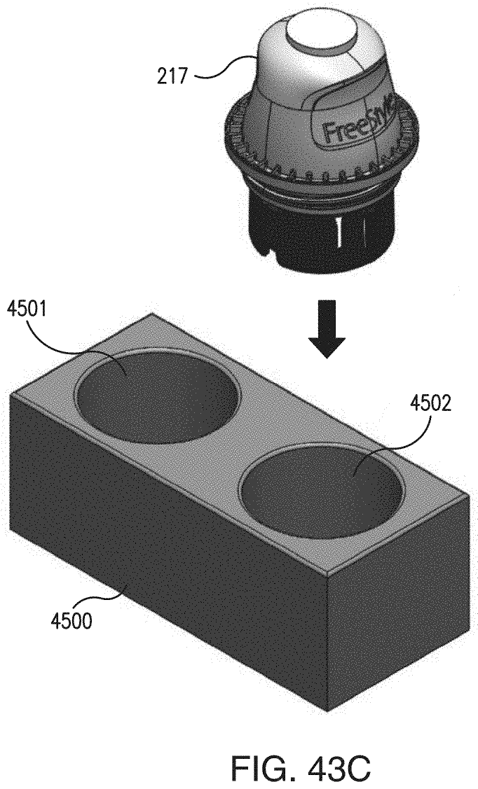

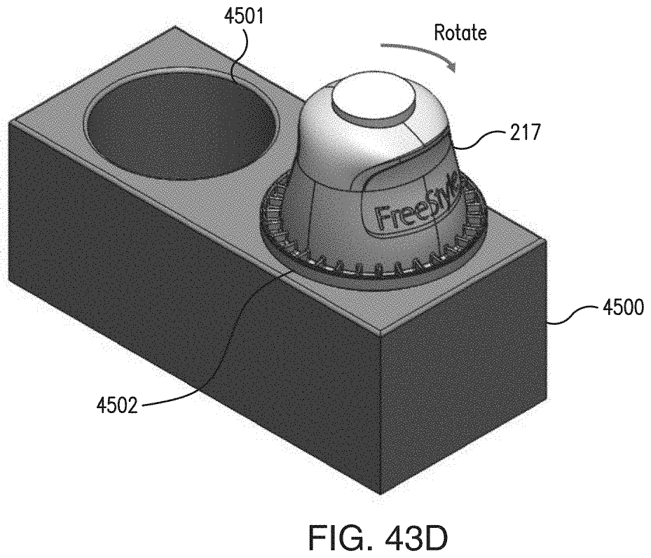

According to aspects of the embodiments of the present disclosure, FIGS. 43A-D illustrate an example embodiment of docking station 4500. While docking station 4000 may be most suitable for two-piece architecture systems, docking station 4500 may be suitable for use with one-piece architecture applicator systems (e.g., applicator 150 as shown in FIGS. 25A-B) and sensor control devices (e.g., sensor control device 9102 as shown in FIGS. 33A-B) as described herein. Similar to docking station 4000, docking station 4500 can include alignment feature 4003, collection chamber 4004, sharp channel 4005 and sheath channels 4006. In contrast to docking station 4000, however, docking station 4500 can include two channels, 4501 and 4502, and applicator 217 is reset prior to being coupled a new unused sensor control device.

As best seen in FIG. 43A, in stage 1, similarly to channel 4002 of docking station 4000, channel 4501 can be used for removing removable plug 217a, inserting reset tool 8000 into applicator 217, and resetting sharp carrier, return spring, and sheath. As best seen in FIGS. 43B-C, in stage 2, channel 4502 can be used to couple a new sensor control device 9102 to reusable applicator 217. A new, unused sensor control device 9102 can be releasably positioned in channel 4502 of docking station 4500 as indicated by the arrow in FIG. 43B. Similar to docking station 4000, channel 4502 of docking station 4500 can include alignment feature configured to rotationally align sensor control device 9102. Subsequently, reset reusable applicator 217 can be placed within and advanced into channel 4502, as indicated by the arrow, until sensor control device 9102 couples to sensor carrier 1022 (e.g., sensor carrier 1022 as seen in FIG. 40A). Furthermore, channel 4502, can include features of applicator cap 9506 disclosed herein. Consequently, when user is ready to reuse applicator 217, user may rotate applicator 217 in direction B′, such that continued rotation of applicator 217 in direction B′ causes sensor cap 9120 to detach from sensor control device 9102 and remain in docking station 4500. As a result, after detachment of sensor cap 9120 from the sensor control device 9102, distal portions of sensor 9112 and sharp 9114 are exposed and sensor control device 9102 is in position for re-firing (re-use).

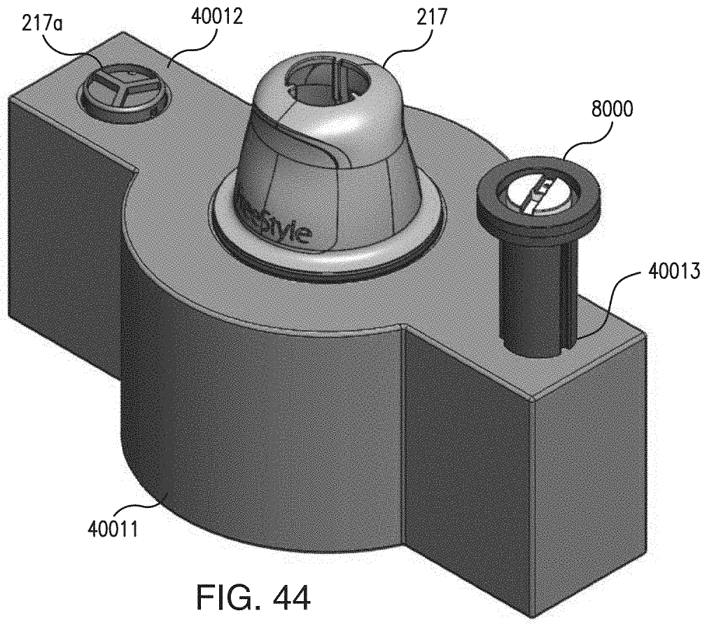

According to aspects of the embodiments of the present disclosure, docking stations can additionally include holders for storage of removable plug 217a and reset tool 8000 when removable plug 217a or reset tool 8000 are not in use. As seen in FIG. 44, docking station 40011 can include holder 40012 to house removable plug 217a when removable plug 217a is not in use (e.g., after removable plug 217a has been removed from applicator 217 during the reset process). Additionally, docking station 40011 can include holder 40013 to house reset tool 8000 when reset tool 8000 is not in use (e.g., after applicator 217 has been reset).

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

A number of deflectable structures are described herein, including but not limited to deflectable detent snaps 1402, deflectable locking arms 1412, sharp carrier lock arms 1524, sharp retention arms 1618, and module snaps 2202. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

### Exemplary Embodiments and Features are Set Out in the Following Numbered Clauses:

1. An assembly for delivery of an analyte sensor comprising:
a reusable applicator configured to deliver a first analyte sensor, the reusable applicator having a proximal portion and a distal portion and including:
a housing;
a sensor carrier configured to releasably receive the first analyte sensor; and
a sharp carrier configured to releasably receive a sharp module and movable between the proximal portion of the reusable applicator and the distal portion of the reusable applicator for delivery of the first analyte sensor from the reusable applicator; and
a reset tool configured to reset the reusable applicator for delivery of another analyte sensor.
2. The assembly of clause 1, wherein the reusable applicator includes a removable plug to access a reset channel accessible.
3. The assembly of clause 1 or 2, further comprising a docking station including a recess to releasably position another analyte sensor and a collection chamber to collect the sharp module.
4. The assembly of clause 3, wherein the docking station includes a first channel to collect the sharp module and a second channel to releasably position another analyte sensor.
5. The assembly of clauses 1 to 4, wherein the reusable applicator further includes a sheath movable between the proximal portion of the reusable applicator and the distal portion of the reusable applicator, and wherein the reset tool comprises a first longitudinal length having:
a first section having a first traverse dimension configured to be inserted into the sharp carrier of the reusable applicator to release the sharp module; and
a second section having a second traverse dimension configured to be inserted into the sheath of the reusable applicator to move the sharp carrier from the proximal portion of the reusable applicator toward the distal portion of the reusable applicator.
6. The assembly of clause 5, wherein the reset tool further comprises a second longitudinal length having a third traverse dimension configured to be inserted into the reusable applicator to move the sheath from the proximal portion of the reusable applicator toward the distal portion of the reusable applicator.
7. The assembly of clause 6, wherein the first longitudinal length is telescopically coupled to the second longitudinal length.
8. The assembly of clause 6 or 7, wherein the second longitudinal length of the reset tool includes a handle portion.
9. The assembly of any of clauses 6 to 8, wherein the third traverse dimension is larger than the second traverse dimension, and the second traverse dimension is larger than the first traverse dimension.
10. The assembly of any of clauses 6 to 9, wherein the second longitudinal length of the reset tool houses a spring.
11. The assembly of any of clauses 1 to 10, wherein the reusable applicator is made of a recyclable material.
12. The assembly of any of clauses 1 to 11, wherein the reusable applicator comprises acetal.
13. The assembly of any of clauses 1 to 12, further comprising a sealable container having a low moisture vapor transition rate to package the reusable applicator.
14. The assembly of any of clauses 1 to 13, further comprising an applicator cap sealingly coupled to the housing with a gasketless seal.
15. A method for delivery of an analyte sensor comprising:
providing a reusable applicator having a proximal portion and a distal portion, a housing, a sensor carrier having a first analyte sensor releasably received therein, and a sharp carrier having a sharp module releasably received therein;
moving the sharp carrier from the proximal portion of the reusable applicator toward the distal portion of the reusable applicator to deliver a first analyte sensor from the reusable applicator; and using a reset tool to reset the reusable applicator for delivery of another analyte sensor.
16. The method of clause 15, further comprising delivering the another analyte sensor from the reusable applicator.
17. The method of clause 15 or 16, wherein using the reset tool includes: inserting the reset tool within a reset channel of the reusable applicator;
advancing the reset tool to release the sharp module releasably received within the sharp carrier of the reusable applicator;
advancing the reset tool to compress a return spring of the reusable applicator by moving the sharp carrier of the reusable applicator from the proximal portion of the reusable applicator toward the distal portion of the reusable applicator; and
advancing the reset tool to move a sheath of the reusable applicator from the proximal portion of the reusable applicator toward the distal portion of the reusable applicator.
18. The method of clause 17, further comprising:
advancing the reusable applicator into a channel of a docking station, the channel releasably positioning another analyte sensor and the docking station including a collection chamber to collect the sharp module;
coupling the another analyte sensor to the sensor carrier; and
releasing the sharp module into the collection chamber.
19. The method of clause 17 or 18, further comprising:
advancing the reusable applicator into a first channel of a docking station including a collection chamber to collect the sharp module;
releasing the sharp module into the collection chamber;
advancing the reusable applicator into a second channel of the docking station releasably positioning another analyte sensor; and
coupling the another analyte sensor to the sensor carrier.
20. The method of any of clauses 17 to 19, further comprising removing a removable plug to access the reset channel.
21. The method of any of clauses 17 to 20, further comprising packaging the reusable applicator into a sealable container for shipment.
22. The method of any of clauses 17 to 21, further comprising removing an applicator cap from the housing, wherein the applicator cap is sealingly coupled to the housing with a gasketless seal.

In summary, an assembly and method for delivery of an analyte sensor including a reusable applicator having a proximal portion and a distal portion are disclosed. The reusable applicator can include a housing, a sensor carrier configured a sensor carrier configured to releasably receive a first analyte sensor, a sharp carrier configured to releasably receive a sharp module and movable between the proximal portion of the reusable applicator and the distal portion of the reusable applicator for delivery of the first analyte sensor from the reusable applicator, and a reset tool configured to reset the reusable applicator for delivery of another analyte sensor.

The description encompasses and expressly envisages methods that are non-surgical, non-invasive methods implemented outside the body. The methods are typically implemented by a user who is not required to be a medical professional.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1-22. (canceled)

23. An applicator for delivering a sensor control device, the applicator comprising:
a housing, comprising a sealing lip;
a sensor carrier coupled to the housing;
a sheath, slidably coupled to the housing to move between an extended position and a collapsed position; and
a cap threadably coupled with the housing,
wherein the cap includes a cavity, wherein the sealing lip of the housing is configured to mate with the cavity to form a gasketless seal interface, and wherein the gasketless seal interface is configured to serve as a moisture barrier.

24. The applicator of claim 23, wherein the sealing lip comprises an axial extension configured to mate with the cavity.

25. The applicator of claim 24, wherein the axial extension of the sealing lip comprises a first axial extension, wherein the cap further comprises a second axial extension and a third axial extension, and wherein the second axial extension and the third axial extension define the cavity.

26. The applicator of claim 25, wherein the first axial extension and the second axial extension are configured to form two radial seals, and wherein the two radial seals are configured to prevent migration of fluids or contaminants across the gasketless seal.

27. The applicator of claim 26, wherein the two radial seals comprise a redundant sealing configuration configured to accommodate tolerance and thermal variations.

28. The applicator of claim 25, wherein a diameter of the cavity is sized to receive a diameter of the first axial extension.

29. The applicator of claim 25, wherein the diameter of the first axial extension at a first height corresponds to the diameter of the cavity at the first height, wherein the diameter of the first axial extension at a second height corresponds to the diameter of the cavity at the second height, and wherein the diameter of the first axial extension at the first height is different from the diameter of the first axial extension at the second height.

30. The applicator of claim 23, wherein the housing comprises a first plurality of threads, and wherein the cap comprises a second plurality of threads configured to engage with the first plurality of threads.

31. The applicator of claim 30, wherein the first plurality of threads comprises a first plurality of curved surfaces, and wherein the second plurality of threads comprises a second plurality of curved surfaces corresponding to the first plurality of curved surfaces.

32. The applicator of claim 31, wherein the first plurality of threads further comprises a first plurality of flat surfaces, and wherein the second plurality of threads further comprises a second plurality of flat surfaces corresponding to the first plurality of flat surfaces.

33. The applicator of claim 30, wherein the first plurality of threads comprises a first plurality of angled surfaces, and wherein the second plurality of threads comprises a second plurality of angled surfaces corresponding to the first plurality of angled surfaces.

34. The applicator of claim 33, wherein the first plurality of threads further comprises a first plurality of flat surfaces, and wherein the second plurality of threads further comprises a second plurality of flat surfaces corresponding to the first plurality of flat surfaces.

35. The applicator of claim 30, wherein the first plurality of threads comprises a first plurality of chamfered surfaces, and wherein the second plurality of threads comprises a second plurality of chamfered surfaces corresponding to the first plurality of chamfered surfaces.

36. The applicator of claim 35, wherein the first plurality of threads further comprises a first plurality of flat surfaces, and wherein the second plurality of threads further comprises a second plurality of flat surfaces corresponding to the first plurality of flat surfaces.

37. The applicator of claim 23, further comprising the sensor control device.

38. The applicator of claim 37, wherein the sensor control device comprises an in vivo glucose sensor, an electronics housing, and sensor electronics disposed within the electronics housing.

39. The applicator of claim 38, wherein the sensor electronics comprise one or more processors, memory, and wireless communication circuitry.

40. The applicator of claim 39, wherein the wireless communication circuitry is configured to transmit data indicative of an in vivo glucose level to a reader device.

41. The applicator of claim 37, further comprising a sharp and a spring,
wherein the applicator is configured to advance the sensor carrier, the sensor control device, and the sharp from a proximal position entirely within the applicator to a distal position, and
wherein the spring is configured to automatically retract the sharp from the distal position to a retracted position entirely within the applicator.

42. The applicator of claim 23, wherein the cap is configured to hold a desiccant component.
